(19) United States
(12) Patent Application Publication
(10) Pub. No.: US 2026/0001962 A1
(43) Pub. Date: Jan. 1, 2026

(54) COMBINATION THERAPY METHODS WITH T-CELL ENGAGING MOLECULES FOR TREATMENT OF PROSTATE CANCER

(71) Applicant: AMGEN INC., Thousand Oaks, CA (US)

(72) Inventors: Tobias EGGERT, San Diego, CA (US); Hosein KOUROS-MEHR, Los Angeles, CA (US)

(73) Assignee: AMGEN INC., Thousand Oaks, CA (US)

(21) Appl. No.: 18/848,857

(22) PCT Filed: Mar. 20, 2023
(86) PCT No.: PCT/US2023/015633

§ 371 Date: Sep. 19, 2024

Related U.S. Application Data
(60) Provisional application No. 63/322,138, filed on Mar. 21, 2022.

(51) Int. Cl. C07K 16/30; A61K 9/00; A61K 31/4166; A61K 31/58; A61K 39/00; A61P 35/04; C07K 16/28

(52) U.S. Cl. CPC C07K 16/3069; A61K 9/0019; A61K 9/0053; A61K 31/4166; A61K 31/58; A61P 35/04; C07K 16/2809; A61K 2039/505; A61K 2039/54; A61K 2039/545; C07K 2317/31; C07K 2317/524; C07K 2317/526; C07K 2317/53; C07K 2317/565

(57) ABSTRACT

The present invention relates to treatment methods for prostate cancer using a combination of an anti-androgen compound and a T-cell engaging molecule that specifically binds to human prostate-specific membrane antigen (PSMA) and human CD3. In particular, the present invention relates to methods for treating prostate cancer, including metastatic castration-resistant prostate cancer, in a patient in need thereof comprising administering to the patient one or more cycles of an anti-androgen compound in combination with a PSMA-targeted T-cell engaging molecule, wherein administration of the first dose of the anti-androgen compound is delayed relative to administration of the first therapeutic dose of the PSMA-targeted T-cell engaging molecule in the first cycle.

### CROSS-REFERENCE TO RELATED APPLICATIONS

This application claims the benefit of U.S. Provisional Application No. 63/322,138, filed Mar. 21, 2022, which is hereby incorporated by reference in its entirety.

### DESCRIPTION OF THE TEXT FILE SUBMITTED ELECTRONICALLY

The present application contains a Sequence Listing, which has been submitted electronically in XML format and is hereby incorporated by reference in its entirety. The computer readable format copy of the Sequence Listing, which was created on Mar. 20, 2023, is named 10004-WO01-SEC_ST26.xml and is 206, 177 bytes in size.

### FIELD OF THE INVENTION

The present invention relates to the fields of immuno-oncology and biopharmaceuticals. In particular, the invention relates to methods of treating prostate cancer, particularly castration-resistant prostate cancer, by administering a T-cell engaging molecule that specifically binds to human prostate-specific membrane antigen (PSMA) and human cluster of differentiation 3 (CD3) in combination with an anti-androgen compound according to specific dosing schedules.

### BACKGROUND OF THE INVENTION

Prostate cancer is the second most frequent malignancy (after lung cancer) diagnosed in men worldwide. In 2018, 1,276,106 new cases of prostate cancer were reported worldwide, and prostate cancer caused 358, 989 deaths (3.8% of all deaths caused by cancer in men) (Rawla, World J Oncol., Vol. 10:63-89, 2019).

Metastasis is a primary cause of morbidity and mortality for prostate cancer. Since 1941, patients diagnosed with metastatic prostate cancer have received continuous androgen-deprivation therapy (ADT) in the form of surgical castration, chemical castration involving luteinizing hormone-releasing hormone agonist (LHRH)-modulating compounds, and/or anti-androgen therapy. Metastatic prostate cancer often develops resistance to ADT (“castration-resistance”) due to increased intratumoral steroidogenesis, altered steroid-transporter expression, increased androgen receptor expression (e.g. androgen receptor amplification), and other mechanisms (Galletti et al., Cancer Treat Rev., Vol. 57:16-27, 2017). Since 2010, several therapies have been approved to treat these patients with metastatic castration-resistant prostate cancer (mCRPC). Two novel hormonal therapies, enzalutamide and abiraterone, have demonstrated significant survival benefits in mCRPC patients. In first line mCRPC, abiraterone plus prednisone improved median OS from 30.3 to 34.7 months compared to placebo plus prednisone. Similarly, enzalutamide improved OS in the first line mCRPC setting (35.3 vs 31.3 months) (Sartor and de Bono, N Engl J Med., Vol. 378:645-657, 2018). Radium-223 also demonstrated survival benefit (14.9 vs 11.3 months) in patients with bone metastases when combined with best standard care, which included older hormonal therapies, radiation, and bisphosphonates. Sipuleucel-T, an autologous cellular immunotherapy, increased median survival by 4.1 months compared with placebo, though PSA and radiographic responses were not observed. Cabazitaxel, a tubulin-binding taxane, increased median survival by 2.4 months compared with mitoxantrone, though many trial participants did not complete treatment due to toxicity. While recently approved therapies have demonstrated survival benefits for patients with mCRPC, drug resistance often complicates the disease course and contributes to relapse and mortality. In addition, the effects of the approved therapies are generally not durable and can be associated with limited tolerability. See, e.g., de Bono et al., Lancet, Vol. 376:1147-54, 2010; Scher et al., N Engl J Med, Vol. 367:1187-97, 2012; Parker et al., N Engl J Med, Vol. 369:213-23, 2013; and Ryan et al., Lancet Oncol, Vol. 16:152-60, 2015.

Emerging PSMA-targeted therapies have shown activity in mCRPC patients (Hofman et al., Lancet Oncol., Vol. 19 (6): 825-833, 2018). PSMA is a 100 kDa type-II integral membrane glycoprotein that is mainly expressed on prostate epithelial cells (Christiansen et al., Prostate, Vol. 55:9-19, 2003, Israeli et al., Cancer Res., Vol. 53:227-230, 1993). PSMA is expressed in the prostate and in a limited number of tissues, including in a subset of renal proximal tubules, some cells of the intestinal brush-border membrane, liver and rare cells in the colonic crypts (O'Keefe et al., Prostate, Vol. 58:200-210, 2004; Chang et al., Cancer Res., Vol. 59:3192-3198, 1999; Troyer et al., Int J Cancer, Vol. 62:552-558, 1995; Israeli et al., Cancer Res., Vol. 54:1807-1811, 1994; Lopes et al., Cancer Res., Vol. 50:6423-6429, 1990; Horoszewicz et al., Anticancer Res., Vol. 7:927-935, 1987). In prostate cancer, expression of PSMA increases with disease progression and is highest in metastatic disease, hormone refractory cases, and higher-grade lesions. There is a strong correlation between a negative prognosis and cell surface expression of PSMA. Consistent with the correlation between PSMA expression and tumor stage, increased levels of PSMA are associated with androgen-independent prostate cancer (Wright et al., Urology, Vol. 48:326-334, 1996; Israeli et al., 1994, supra). Immunohistochemical analysis revealed relatively intense and homogeneous expression of PSMA within metastatic lesions localized to lymph nodes, bone, soft tissue, and lungs compared with benign prostatic tissues (Chang et al., Urology, Vol. 57:1179-1183, 2001; Sweat et al., Urology, Vol. 52:637-640, 1998; Murphy et al., Cancer, Vol. 78:809-818, 1996). The restricted expression of PSMA and its up-regulation in advanced carcinoma and metastatic disease has made PSMA an attractive target for the development of therapies for prostate cancer.

Advanced prostate cancer is a heterogeneous disease that frequently develops resistance to single agent therapy (Carm et al., Sci Rep., Vol. 9:13579, 2019). Combination therapy is therefore warranted to overcome disease heterogeneity and resistance to therapy. Anti-androgen therapies, such as enzalutamide and abiraterone, have been reported to upregulate expression of PSMA on prostate cancer cells (see, e.g., Deegen et al., Clin. Cancer Res., Vol. 27:2928-2937, 2021; Aggarwal et al., Eur. Urol. Oncol., Vol. 1:78-82, 2018; Emmett et al., J Nucl. Med., Vol. 60:950-954, 2019). PSMA-targeted T-cell engagers are a new class of immunotherapies that engage a patient's own T cells and redirect them to kill PSMA-expressing cancer cells. Combination therapy with an anti-androgen compound that upregulates expression of PSMA on cancer cells with a PSMA-targeted T-cell engager may provide a synergistic anti-tumor effect. Indeed, in vitro experiments have shown that enzalutamide enhances cytotoxicity of prostate cancer cells induced by a PSMA-targeted T-cell engager (Deegen et al., 2021, supra). However, the side effect profile associated with such a combination therapy in human patients is not well understood. Therefore, there is a need in the art for safe and effective approaches for administration of anti-androgen compounds in combination with PSMA-targeted immunotherapies to treat prostate cancer, particularly mCRPC.

### SUMMARY OF THE INVENTION

The present invention is based, in part, on the identification of dosing schedules for anti-androgen compounds in combination with PSMA-targeted T-cell engagers that improve the safety and tolerability of the combination therapy while maintaining optimal anti-tumor efficacy. Accordingly, in one embodiment, the present invention provides a method for treating prostate cancer in a patient in need thereof, comprising administering to the patient one or more cycles of an anti-androgen compound in combination with a T-cell engaging molecule that specifically binds to human PSMA and human CD3, wherein a first cycle comprises administering a first dose of the anti-androgen compound at least 3 days after administering a first therapeutic dose of the T-cell engaging molecule.

In certain embodiments, the first cycle (also referred to herein as the initiation cycle) comprises administering a first dose of an anti-androgen compound about 4 days to about 10 days or about 5 days to about 9 days after administering a first therapeutic dose of a PSMA-targeted T-cell engaging molecule. For instance, in some embodiments, the first cycle comprises administering a first dose of an anti-androgen compound about 5 days, about 6 days, or about 7 days after administering a first therapeutic dose of a PSMA-targeted T-cell engaging molecule.

In some embodiments of the methods of the invention, the first cycle further comprises administering to the patient one or more priming doses of the T-cell engaging molecule prior to administering the first therapeutic dose of the T-cell engaging molecule. Such priming doses may, in some embodiments, be lower than therapeutic doses of the T-cell engaging molecule but sufficient to induce T-cell activation in a patient to prime or prepare the patient to receive higher doses of the T-cell engaging molecule such that administration of the higher doses results in a reduced number or severity of adverse events, like cytokine release syndrome. In some embodiments in which one or more priming doses of the PSMA-targeted T-cell engaging molecule is administered to the patient in the first cycle, the first therapeutic dose of the T-cell engaging molecule can be administered about 1 day to about 21 days, about 1 day to about 14 days, or about 1 day to about 7 days after administration of the first priming dose of the PSMA-targeted T-cell engaging molecule to the patient. In certain embodiments, the first therapeutic dose of the PSMA-targeted T-cell engaging molecule is administered about 3 days, about 4 days, about 5 days, about 6 days, or about 7 days after administration of the first priming dose of the PSMA-targeted T-cell engaging molecule.

In various embodiments of the methods of the invention, the PSMA-targeted T-cell engaging molecule is administered to the patient by intravenous infusion or subcutaneous injection. In some embodiments, one or more doses of the PSMA-targeted T-cell engaging molecule is administered to the patient by a continuous intravenous infusion over an extended period of time, such as over a period of I day to 7 days. In some embodiments, one or more doses of the PSMA-targeted T-cell engaging molecule is administered to the patient by a bolus intravenous infusion (also referred to herein as a short intravenous infusion) over a period of about 30 minutes to about 90 minutes. In certain embodiments, the first dose of the PSMA-targeted T-cell engaging molecule in the first cycle is administered by continuous intravenous infusion over a period of 1 day to 7 days, for example over a period of 3 days (i.e. 72-hour infusion period). In some such embodiments, the first dose may be a priming dose and less than the amount of a therapeutic dose. In related embodiments, one or more therapeutic doses of the PSMA-targeted T-cell engaging molecule may be subsequently administered to the patient by a bolus intravenous infusion in the first cycle.

In some embodiments, the anti-androgen compound is administered to the patient orally. Depending on the anti-androgen compound employed, the anti-androgen compound is administered to the patient once per day, twice per day, or three times per day in an oral dosage form, such as a tablet or capsule. The anti-androgen compound may be abiraterone, abiraterone acetate, ketoconazole, flutamide, bicalutamide, nilutamide, enzalutamide, apalutamide, or darolutamide. In certain embodiments, the anti-androgen compound administered to the patient according to the methods of the invention is enzalutamide, abiraterone, abiraterone acetate, apalutamide, or darolutamide. In one embodiment, the anti-androgen compound is enzalutamide. Enzalutamide may be administered to the patient orally at a dose of about 160 mg once per day. In another embodiment, the anti-androgen compound is abiraterone or abiraterone acetate. In some such embodiments, abiraterone or abiraterone acetate is administered to the patient orally at a dose of about 1,000 mg once per day. In yet another embodiment, the anti-androgen compound is apalutamide, which can be administered to the patient orally at a dose of about 240 mg once per day. In still another embodiment, the anti-androgen compound is darolutamide. In some embodiments, darolutmaide is administered to the patient orally at a dose of about 600 mg twice per day.

The first cycle may have a duration from about 14 days to about 56 days or from about 21 days to about 28 days. In certain embodiments of the methods of the invention, the first cycle has a duration of about 28 days. In such embodiments, the first cycle may comprise administering a PSMA-targeted T-cell engaging molecule on days 1, 8, 15, and 22 of the cycle and administering an anti-androgen compound once per day on each of days 15 to 28 of the cycle. In other embodiments, the first cycle may comprise administering a PSMA-targeted T-cell engaging molecule on days 1 and 15 of the cycle and administering an anti-androgen compound once per day on each of days 15 to 28 of the cycle. In certain other embodiments in which the first cycle is about 28 days, the first cycle may comprise administering a first dose (e.g. a priming dose) of a PSMA-targeted T-cell engaging molecule by continuous intravenous infusion over days 1 to 3 of the cycle, administering a therapeutic dose of the PSMA-targeted T-cell engaging molecule by a bolus intravenous infusion on days 8 and 22 of the cycle, and administering an anti-androgen compound orally once per day on each of days 15 to 28 of the cycle. In such embodiments, the first dose (e.g. a priming dose) of the PSMA-targeted T-cell engaging molecule may be from about 30 μg to about 300 μg or from about 30 μg to about 150 μg and the therapeutic dose of the PSMA-targeted T-cell engaging molecule may be from about 90 μg to about 1.8 mg or from about 100 μg to about 600 μg. In one particular embodiment, the first dose (e.g. a priming dose) of the PSMA-targeted T-cell engaging molecule is about 90 μg and the therapeutic dose is about 150 μg or 300 μg.

In some embodiments, the methods further comprise administering one or more maintenance cycles of an anti-androgen compound in combination with a PSMA-targeted T-cell engaging molecule to the patient. In one embodiment, the maintenance cycle comprises administering the anti-androgen compound orally once per day on each day of the cycle and administering a therapeutic dose of the PSMA-targeted T-cell engaging molecule by a bolus intravenous infusion once every 7 days or once every 14 days. The duration of the maintenance cycle may be about 21 days to about 42 days, about 28 days to about 56 days, or from about 21 days to about 28 days. In certain embodiments, the maintenance cycle has a duration of about 28 days. The maintenance cycle may be administered the following day after completing the first cycle, for example with no treatment-free periods between the first cycle and the maintenance cycle. In other embodiments, the maintenance cycle is administered about 7 days following the completion of the first cycle—i.e. there is a 7-day treatment-free period between the first cycle and the maintenance cycle. A patient may receive multiple maintenance cycles, such as 2, 3, 4, 5, 6, 7, 8, 9, 10, 11, or 12 or more maintenance cycles. In some embodiments, maintenance cycles are administered to the patient until the patient responds to treatment, for example achieves a complete response.

In certain embodiments, the prostate cancer to be treated according to the methods of the invention is metastatic prostate cancer. Accordingly, the patient to be treated according to the methods of the invention has or is diagnosed with metastatic prostate cancer. The metastatic prostate cancer may be hormone-sensitive, or it may be resistant to hormone therapy. Thus, in one embodiment, the patient to be treated according to the methods of the invention has or is diagnosed with metastatic castration-resistant prostate cancer.

Prostate cancer patients to be treated according to the methods of the invention may have received one or more prior therapies for prostate cancer and have failed or become intolerant, refractory, or resistant to one or more of these prior therapies. For example, in some embodiments, the patients have failed or are intolerant, refractory, or resistant to one or more chemotherapy regimens, such as taxane-containing chemotherapy regimens. Additionally or alternatively, the patients have failed or are intolerant, refractory, or resistant to one or more anti-androgen compounds, such as abiraterone, enzalutamide, apalutamide, or darolutamide. In such embodiments, the anti-androgen compound to be administered to the patient in combination with the PSMA-targeted T-cell engaging molecule according to the methods of the invention is preferably a different anti-androgen compound than the anti-androgen compound the patient received previously. In certain embodiments, the patients to be treated according to the methods of the invention have failed or are intolerant, refractory, or resistant to a radioligand therapy, such as ¹⁷⁷Lu-PSMA-617.

In any embodiments of the methods disclosed herein, the PSMA-targeted T-cell engaging molecule administered to the patient specifically binds to PSMA and CD3, preferably human PSMA and human CD3. Thus, the PSMA-targeted T-cell engaging molecule comprises a first domain that specifically binds to human PSMA and a second domain that specifically binds to human CD3. The binding domains can comprise structural elements from antibodies or antigen-binding fragments thereof, such as heavy and light chain variable regions. In one embodiment, either or both of the binding domains of the PSMA-targeted T-cell engaging molecule used in the methods of the invention is a single-chain variable fragment (scFv). In some embodiments of the methods described herein, the PSMA-targeted T-cell engaging molecules further comprise an Fc domain having one or more immunoglobulin Fc monomers. In such embodiments, the Fc domain can be a single-chain Fc domain.

In certain embodiments, the PSMA-targeted T-cell engaging molecule administered to the patient according to the methods of the invention comprises, in an amino to carboxyl order: (i) a first domain that specifically binds to human PSMA, (ii) a second domain that specifically binds to human CD3, and (iii) an Fc domain comprising two Fc monomers, each monomer comprising an immunoglobulin hinge region, a CH2 domain, and a CH3 domain, wherein said two Fc monomers are fused to each other via a peptide linker. In one embodiment, the first domain comprises a first immunoglobulin heavy chain variable region (VH1) comprising a CDRH1 having the sequence of SEQ ID NO: 14, a CDRH2 having the sequence of SEQ ID NO: 16, and a CDRH3 having the sequence of SEQ ID NO: 20, and a first immunoglobulin light chain variable region (VL1) comprising a CDRL1 having the sequence of SEQ ID NO: 5, a CDRL2 having the sequence of SEQ ID NO: 8, and a CDRL3 having the sequence of SEQ ID NO: 9. In a related embodiment, the second domain comprises a second immunoglobulin heavy chain variable region (VH2) comprising a CDRH1 having the sequence of SEQ ID NO: 49, a CDRH2 having the sequence of SEQ ID NO: 55, and a CDRH3 having the sequence of SEQ ID NO: 60, and a second immunoglobulin light chain variable region (VL2) comprising a CDRL1 having the sequence of SEQ ID NO. 43, a CDRL2 having the sequence of SEQ ID NO. 44, and a CDRL3 having the sequence of SEQ ID NO: 47. In some embodiments, the first domain (e.g. anti-PSMA domain) of the PSMA-targeted T-cell engaging molecule comprises a heavy chain variable region comprising the sequence of SEQ ID NO: 33 and a light chain variable region comprising the sequence of SEQ ID NO: 30. In these and other embodiments, the second domain (e.g. anti-CD3 domain) of the PSMA-targeted T-cell engaging molecule comprises a heavy chain variable region comprising the sequence of SEQ ID NO: 72 and a light chain variable region comprising the sequence of SEQ ID NO: 70.

The PSMA-targeted T-cell engaging molecule administered to patients according to the methods of the invention may comprise (i) a first domain that specifically binds to human PSMA and has the amino acid sequence of SEQ ID NO: 104, (ii) a second domain that specifically binds to human CD3 and has the amino acid sequence of SEQ ID NO: 116, and (iii) an Fc domain comprising two Fc monomers each having the amino acid sequence of SEQ ID NO: 124, wherein said two Fc monomers are fused to each other via a peptide linker. In related embodiments, the Fc domain of the PSMA-targeted T-cell engaging molecule comprises the amino acid sequence of SEQ ID NO: 132. In certain embodiments, the PSMA-targeted T-cell engaging molecule used in the methods of the invention is a single chain polypeptide or single chain fusion protein. Thus, any of the single chain polypeptides described in Table 6 herein are suitable for use in the methods of the invention. In a preferred embodiment, the PSMA-targeted T-cell engaging molecule administered to a patient according to the methods of the invention is a single chain polypeptide comprising the amino acid sequence of SEQ ID NO. 140 (e.g. acapatamab).

The present invention also provides pharmaceutical compositions of PSMA-targeted T-cell engaging molecules for use in combination with an anti-androgen compound in the methods described herein. The pharmaceutical compositions can comprise one or more pharmaceutically acceptable diluents, carriers, or excipients, including buffers, surfactants, and stabilizing agents. In certain embodiments, the pharmaceutical compositions comprise a PSMA-targeted T-cell engaging molecule, a buffer, a surfactant, and a stabilizing agent. In one embodiment, the pharmaceutical composition comprises a PSMA-targeted T-cell engaging molecule (e.g. single chain polypeptide comprising the amino acid sequence of SEQ ID NO. 140), a glutamate buffer, polysorbate 20 or polysorbate 80, and sucrose, at a pH of about 4.0 to about 4.4. In some embodiments, the pharmaceutical compositions may be lyophilized and reconstituted prior to administration to a patient.

The use of PSMA-targeted T-cell engaging molecules in combination with anti-androgen compounds in any of the methods disclosed herein or for preparation of medicaments for administration according to any of the methods disclosed herein is specifically contemplated. For instance, the present invention includes a PSMA-targeted T-cell engaging molecule for use in combination with an anti-androgen compound in a method for treating prostate cancer in a patient in need thereof, wherein the method comprises administering a first cycle of the PSMA-targeted T-cell engaging molecule and the anti-androgen compound, said first cycle comprising administering a first dose of the anti-androgen compound at least 3 days after administering a first therapeutic dose of the T-cell engaging molecule. The present invention also includes the use of a PSMA-targeted T-cell engaging molecule for the manufacture of a medicament for the treatment of prostate cancer in a patient in need thereof, wherein the treatment comprises administering to the patient a first cycle of the PSMA-targeted T-cell engaging molecule and the anti-androgen compound, said first cycle comprising administering a first dose of the anti-androgen compound at least 3 days after administering a first therapeutic dose of the T-cell engaging molecule.

### DETAILED DESCRIPTION

The present invention is based, in part, on the finding that delaying the initiation of treatment with an anti-androgen compound for an extended period of time following administration of a PSMA-targeted T-cell engaging molecule significantly reduced the number of serious adverse events and dose limiting toxicities observed with the combination therapy as compared to a regimen when the PSMA-targeted T-cell engaging molecule and anti-androgen compound were first administered to the patient at the same time. See Example 1. In addition, the anti-tumor efficacy of the combination was comparable between the two regimens. These results are somewhat surprising for at least two reasons. Based on the mechanisms of action of the anti-androgen compound and the PSMA-targeted T-cell engaging molecule, no overlapping toxicity was expected because the potential adverse events for each molecule were distinct. For example, the most frequent adverse events for anti-androgen compounds, such as enzalutamide, are hot flashes and fatigue, whereas the most common adverse event for PSMA-targeted T-cell engaging molecules is cytokine release syndrome (CRS). Second, anti-androgen compounds have been reported to up-regulate expression of PSMA on prostate cancer cells suggesting that an anti-androgen compound should be administered prior to or at least simultaneously with a PSMA-targeted T-cell engaging molecule to observe a synergistic effect on tumor cell cytotoxicity. However, delaying the initiation of treatment with an anti-androgen compound did not significantly impact the efficacy of the combination therapy (see Example 1). Therefore, the present invention provides methods for administering anti-androgen compounds in combination with PSMA-targeted T-cell engagers that improve the safety and tolerability of the combination therapy while maintaining optimal anti-tumor efficacy in patients with prostate cancer. Accordingly, in certain embodiments, the present invention provides methods for treating prostate cancer in a patient in need thereof, comprising administering to the patient one or more cycles of an anti-androgen compound in combination with a T-cell engaging molecule that specifically binds to human PSMA and human CD3, wherein a first cycle comprises administering a first dose of the anti-androgen compound at least 3 days after administering a first therapeutic dose of the T-cell engaging molecule.

Prostate cancer is one of the most common types of cancer in men and occurs when cells in the prostate gland begin to grow out of control. Most forms of prostate cancer are adenocarcinomas, which are tumors formed from glandular cells. Other forms of prostate cancer include small cell carcinomas, neuroendocrine tumors, transitional cell carcinomas, and sarcomas. Prostate cancer is initially confined to the prostate gland but can metastasize and spread to other tissues. Metastatic prostate cancer can be divided into two primary types: a first type where the cancer bas not been treated with androgen deprivation therapy (“metastatic hormone-sensitive prostate cancer” or mHSPC) and a second type where the cancer is resistant to androgen deprivation therapy (“metastatic castration-resistant prostate cancer” or mCRPC). In prostate cancer, expression of PSMA increases with disease progression and is highest in metastatic disease, hormone refractory cases, and higher-grade lesions.

In its early stages, prostate cancer may not cause any signs or symptoms. As the disease progresses, signs and symptoms of prostate cancer can include incontinence, trouble urinating, blood in semen or urine, erectile dysfunction, pain in pelvic area or bones, or weakness in legs or feet. Prostate cancers are typically diagnosed and monitored by one or more tests conducted on a sample (e.g. blood, serum, plasma, semen, tissue) from a subject or patient suspected of having or developing prostate cancer. A sample can be any biological sample obtained from a human patient and can include body fluids, such as blood, serum, plasma, semen, and urine, and tissues, such as prostate tissue, lymph nodes, or tumor biopsies. A common test used to screen and/or monitor for prostate cancer is the prostate-specific antigen (PSA) blood test. Elevation of PSA in the blood (e.g. serum or plasma) can be an indicator of the presence or progression of prostate cancer. Another test commonly used to detect or monitor prostate cancer is a prostate tissue biopsy. The prostate tissue biopsy sample is evaluated for the presence of abnormal or cancerous cells. If cancerous cells are present, the prostate cancer may be assigned a grade based on the Gleason score grading system or other grading system, which assigns a grade based on how abnormal the cells appear as compared to normal cells. Gleason scores can range from 2 (nonaggressive cancer) to 10 (very aggressive cancer). Prostate cancer may also be diagnosed or monitored using imaging tests including, but not limited to, magnetic resonance imaging (MRI), computed tomography (CT), positron emission tomography (PET) with radioactive tracers, such as ⁶⁸Gallium-PSMA-11, piflufolastat F-18 (a.k.a. ¹⁸F-DCFPyL), or ¹⁸F-flurodeoxyglucose, or bone scans (e.g. bone scintigraphy with ^{98m}technetium-labeled radiotracers).

In certain embodiments, the patients to be treated according to the methods of the invention have or are diagnosed with prostate cancer. In such embodiments, the prostate cancer is PSMA positive—that is the tumors or cancer cells express PSMA as determined by standard immunohistochemical tests of biopsy samples or PSMA imaging methods. In some embodiments, the patients to be treated according to the methods of the invention have blood PSA levels of 4 ng/ml or greater. In other embodiments, the patients to be treated according to the methods of the invention have blood PSA levels of 10 ng/ml or greater. In still other embodiments, the patients to be treated according to the methods of the invention have blood PSA levels of 1 ng/ml or greater, wherein the PSA levels have increased on at least two successive occasions at least a week apart. In some embodiments, the patients to be treated according to the methods of the invention have prostate cancer with a Gleason score of 7. In other embodiments, the patients to be treated according to the methods of the invention have prostate cancer with a Gleason score of 8. In yet other embodiments, the patients to be treated according to the methods of the invention have prostate cancer with a Gleason score of 9 or 10 In some embodiments, the patients to be treated according to the methods of the invention may have been newly diagnosed with prostate cancer. Some patients in need of treatment may have been diagnosed with hormone-sensitive prostate cancer or castration-resistant prostate cancer.

In some embodiments, the patients to be treated according to the methods of the invention have or are diagnosed with metastatic prostate cancer. The metastatic prostate cancer can be hormone sensitive or resistant to hormone therapy. Patients diagnosed with metastatic prostate cancer have evidence of cancerous prostate cells outside the prostate gland, such as in lymph nodes, bones, or other organs, most commonly liver, lung, or brain. Evidence of spread of the cancer cells is typically detected by the presence of tumors or lesions in other tissues by one or more of the imaging methods described above, such as CT, MRI, PET, or bone scans. In some embodiments, the patients to be treated according to the methods of the invention have evidence of progressive prostate cancer as shown by progression of lymph node or visceral tumors as defined by Response Evaluation Criteria in Solid Tumors (RECIST) 1.1, optionally with Prostate Cancer Working Group 3 (PCWG3) modifications (see Eisenhauer et al., European Journal of Cancer, Vol. 45:228-247, 2009; Scher et al., J. Clin, Oncol, Vol. 34:1402-1418, 2016). In other embodiments, the patients to be treated according to the methods of the invention have evidence of progressive prostate cancer as shown by the appearance of two or more new bone lesions as determined by a bone scan (e.g. bone scintigraphy with ^{98m}technetium-labeled radiotracers).

In certain embodiments, the patients to be treated according to the methods of the invention have or are diagnosed with metastatic castration-resistant prostate cancer (mCRPC). mCRPC is diagnosed when the cancer progresses in patients with metastatic disease even though the patients have testosterone levels at or below the testosterone levels achieved by androgen deprivation therapy. In some embodiments, patients who have been diagnosed with mCRPC may have failed, become refractory to, or have relapsed following treatment with an androgen deprivation therapy. Androgen deprivation therapy includes surgical castration (e.g. bilateral orchiectomy), chemical castration with LHRH agonists or antagonists (e.g. leuprolide, goserelin, triptorelin, histrelin, relugolix, or degarelix), or treatment with anti-androgen compounds, such as androgen biosynthesis inhibitors (e.g. abiraterone, ketoconazole), or androgen receptor antagonists (e.g. flutamide, bicalutamide, nilutamide, enzalutamide, apalutamide, or darolutamide). In some embodiments, patients to be treated according to the methods of the invention have total serum testosterone levels of 50 ng/dL (1.7 nmol/L) or less. In other embodiments, patients to be treated according to the methods of the invention have total serum testosterone levels of 20 ng/dL (0.7 nmol/L) or less. In certain embodiments, patients to be treated according to the methods of the invention have undergone a bilateral orchiectomy. In other embodiments, patients to be treated according to the methods of the invention are receiving a continuous therapeutic regimen of a LHRH agonist or antagonist. In still other embodiments, patients to be treated according to the methods of the invention have undergone a bilateral orchiectomy and are receiving a continuous therapeutic regimen of a LHRH agonist or antagonist.

Androgen receptor (AR) signaling is altered in patients with castration-resistant prostate cancer (CRPC) and thus the patients' tumors develop various mutations in genes encoding proteins in the androgen receptor signaling pathway (see Sartor and de Bono, N Engl J Med., Vol. 378:645-657, 2018). Such mutations include mutations in the AR, FOXA1, ZBTB16, and SPOP genes along with mutations in genes involved in AKT signaling, DNA repair, and tumor suppression, such as PTEN, ET5, BRCA2, ATM, and CHEK2. Accordingly, diagnosis of CRPC or mCRPC can be supplemented by gene-expression profiling or genotyping to confirm an initial diagnosis and/or identify a subtype of CRPC or mCRPC.

Administration of a PSMA-targeted T-cell engaging molecule in combination with an anti-androgen compound according to the methods of the invention is for the treatment of prostate cancer or other PSMA-expressing cancers or tumors. The term “treatment” or “treat” as used herein refers to the application or administration of the T-cell engaging molecule in combination with an anti-androgen compound to a patient who has or is diagnosed with prostate cancer or other PSMA positive malignancy, has a symptom of prostate cancer or other PSMA positive malignancy, is at risk of developing prostate cancer or other PSMA positive malignancy, or has a predisposition to prostate cancer or other PSMA positive malignancy for the purpose of curing, healing, alleviating, relieving, altering, ameliorating, or improving prostate cancer or other PSMA positive malignancy, one or more symptoms of prostate cancer or other PSMA positive malignancy, the risk of developing prostate cancer or other PSMA positive malignancy, or predisposition toward prostate cancer or other PSMA positive malignancy. The term “treatment” encompasses any improvement of the disease in the patient, including the slowing or stopping of the progression of prostate cancer or other PSMA positive malignancy in the patient, a decrease in the number or severity of the symptoms of prostate cancer or other PSMA positive malignancy, or an increase in frequency or duration of periods where the patient is free from the symptoms of prostate cancer or other PSMA positive malignancy. The term “patient” includes human patients.

In certain embodiments of the methods of the invention, administration of the PSMA-targeted T-cell engaging molecule in combination with the anti-androgen compound reduces blood levels (e.g. serum or plasma levels) of PSA in the patient by at least about 10%, at least about 20%, at least about 30%, at least about 40%, at least about 50%, at least about 60%, at least about 70%, at least about 75%, at least about 80%, at least about 85%, at least about 90%, or at least about 95%, or about 100% relative to the blood levels of PSA in the patient prior to the start of the treatment (i.e. prior to the administration of the PSMA-targeted T-cell engaging molecule and anti-androgen compound). In some embodiments, administration of the PSMA-targeted T-cell engaging molecule in combination with the anti-androgen compound reduces PSA blood levels in the patient by 30% or greater relative to the PSA blood levels in the patient prior to the start of treatment (i.e. PSA30 response). In other embodiments, administration of the PSMA-targeted T-cell engaging molecule in combination with the anti-androgen compound reduces PSA blood levels in the patient by 50% or greater relative to the PSA blood levels in the patient prior to the start of treatment (i.e. PSA50 response). In yet other embodiments, administration of the PSMA-targeted T-cell engaging molecule in combination with the anti-androgen compound reduces PSA blood levels in the patient by 70% or greater relative to the PSA blood levels in the patient prior to the start of treatment (i.e. PSA70 response). In still other embodiments, administration of the PSMA-targeted T-cell engaging molecule in combination with the anti-androgen compound reduces PSA blood levels in the patient by 90% or greater relative to the PSA blood levels in the patient prior to the start of treatment (i.e. PSA90 response). In certain embodiments of the methods of the invention, administration of the PSMA-targeted T-cell engaging molecule in combination with the anti-androgen compound produces a PSA50 response in at least about 20%, at least about 30%, at least about 40%, at least about 50%, at least about 60%, at least about 70%, at least about 75%, at least about 80%, at least about 85%, at least about 90%, or at least about 95% of prostate cancer patients.

In some embodiments of the methods of the invention, administration of the PSMA-targeted T-cell engaging molecule in combination with the anti-androgen compound induces a complete response, a partial response, or a stable disease response in at least about 20%, at least about 30%, at least about 40%, at least about 50%, at least about 60%, at least about 70%, at least about 75%, at least about 80%, at least about 85%, at least about 90%, or at least about 95% of prostate cancer patients with measurable tumors or lesions prior to the start of treatment as determined by RECIST 1.1 criteria, optionally with PCWG3 modifications (see Eisenhauer et al., European Journal of Cancer, Vol. 45:228-247, 2009; Scher et al., J. Clin, Oncol, Vol. 34:1402-1418, 2016). A complete response (CR) in the context of the invention refers to the condition in which all target lesions have disappeared (i.e. no longer detectable) and any pathological lymph nodes have a reduction in the short axis to less than 10 mm. A partial response (PR) refers to the condition in which there is at least a 30% decrease in the sum of diameters of target lesions relative to the sum of the diameters prior to the start of treatment Stable disease (SD) refers to the condition where the target lesions have not reduced sufficiently to qualify as a PR but have not increased sufficiently to qualify as progressive disease (PD). PD refers to the condition in which there is the appearance of one or more new target lesions or there is at least 20% increase in the sum of diameters of target lesions relative to the smallest sum of diameters occurring previously and an absolute increase of the sum of diameters of at least 5 mm.

Efficacy of the therapeutic regimens described herein can also be assessed in terms of reduction of PSMA-positive tumor burden as assessed by ⁶⁸Ga-PSMA-11 PET/CT imaging, piflufolastat F-18 (¹⁸F-DCFPyL) PET/CT imaging, or imaging with another PSMA radiographic PET tracer relative to PSMA-positive tumor burden prior to the start of treatment, percentage of patients achieving a circulating tumor cell (CTC) response, duration of response to treatment, time to progression of disease, progression-free survival (PFS), and overall survival (OS). In certain embodiments, administration of the PSMA-targeted T-cell engaging molecule in combination with the anti-androgen compound according to the methods of the invention increases the duration of response to treatment, time to progression of disease, PFS, and/or OS as compared to the duration of response to treatment, time to progression of disease, PFS, and/or OS observed for a standard chemotherapy regimen (e.g. a taxane chemotherapy regimen) or standard androgen deprivation therapy regimen.

Generally, the methods of the invention comprise administering to the patient a PSMA-targeted T-cell engaging molecule in combination with an anti-androgen compound in one or more treatment cycles. A “treatment cycle” or “cycle” refers to a period of administration of the T-cell engaging molecule and anti-androgen compound each at specific dosages and dosing intervals. According to the methods of the invention, a patient can receive multiple treatment cycles (e.g. 2, 3, 4, 5, 6, 7, 8, 9, 10, 11, 12, 13, 14 or more cycles). The treatment cycles can be administered to the patient consecutively with no break or period without administration of the T-cell engaging molecule or anti-androgen compound between the cycles. Alternatively, a period without administration of either the T-cell engaging molecule, anti-androgen compound, or both (e.g. a “treatment-free period” or “break”) can be employed between the treatment cycles. The length of the treatment-free period can be adjusted based on the patient's characteristics and/or response to treatment.

Each of the treatment cycles entails administering a PSMA-targeted T-cell engaging molecule in combination with an anti-androgen compound. The term “in combination” or “combination therapy” as used herein refers to the manner of administration of the two compounds such that a therapeutic effect of the PSMA-targeted T-cell engaging molecule coincides at one or more time periods with a therapeutic effect of the anti-androgen compound. Except for the first dose of the anti-androgen compound and the first therapeutic dose of the PSMA-targeted T-cell engaging molecule in the first treatment cycle as described in more detail herein, the dosing of the PSMA-targeted T-cell engaging molecule and the dosing of the anti-androgen compound can occur in a substantially simultaneous manner or in a sequential manner (i.e. each compound is administered on a different day in any order). Substantially simultaneous administration includes concurrent administration and can be accomplished by administering a single formulation comprising both compounds (e.g. a single IV bag containing both compounds) or concurrently administering (e.g. on the same day) separate formulations containing each of the compounds. The anti-androgen compounds need not be administered at the same dosing frequency or dosing interval as the PSMA-targeted T-cell engaging molecule. In general, the anti-androgen compound will be administered at a dose and/or on a time schedule determined for that compound. The anti-androgen compound can be administered on the same day or different days of a treatment cycle as the PSMA-targeted T-cell engaging molecule. Exemplary doses and dosing schedules for the PSMA-targeted T-cell engaging molecule and anti-androgen compound for administration according to the methods of the invention are provided herein.

In certain embodiments, the efficacy of the combination therapy as assessed by any of the methods described above is greater than the efficacy of the PSMA-targeted T-cell engaging molecule monotherapy and the efficacy of the anti-androgen compound monotherapy. For instance, in some embodiments, administration of the PSMA-targeted T-cell engaging molecule in combination with the anti-androgen compound produces a PSA50 response in a greater proportion of prostate cancer patients than that produced with monotherapy of either compound alone. In other embodiments, administration of the PSMA-targeted T-cell engaging molecule in combination with the anti-androgen compound produces a greater number of CRs, PRs, and/or SD in prostate cancer patients with measurable tumors or lesions as determined by RECIST 1.1 criteria, optionally with PCWG3 modifications, as compared to the number of such responses resulting from monotherapy with either compound alone. In still other embodiments, administration of the PSMA-targeted T-cell engaging molecule in combination with the anti-androgen compound increases the duration of response to treatment, time to progression of disease, PFS, and/or OS as compared to the duration of response to treatment, time to progression of disease, PFS, and/or OS observed with a monotherapy regimen of either compound alone. In some embodiments, administration of the PSMA-targeted T-cell engaging molecule in combination with the anti-androgen compound results in a synergistic increase in anti-tumor efficacy as assessed by any of the methods described above (e.g. PSA response, RECIST 1.1. response, PSMA tumor burden by PET/CT, CTC response, etc.).

In preferred embodiments of the methods of the invention, the first treatment cycle (also referred to as the initiation cycle) comprises administering a first therapeutic dose of the PSMA-targeted T-cell engaging molecule followed by a specified delay period, and then administering a first dose of the anti-androgen compound. As described in the Examples herein, when the initiation of treatment with an anti-androgen compound was delayed until about 7 days after administration of the first therapeutic dose of the PSMA-targeted T-cell engaging molecule, the number and severity of adverse events was significantly reduced as compared to when the anti-androgen treatment was initiated at the same time as administration of the first dose of the PSMA-targeted T-cell engaging molecule. Without wishing to be bound by theory, because CRS events are most severe and most frequently occur with the first administration of a therapeutic dose of the PSMA-targeted T-cell engaging molecule, it is believed that delaying initiation of treatment with an anti-androgen compound until CRS symptoms resolve or are ameliorated following administration of the therapeutic dose (e.g. at least 3 days) improves the safety and tolerability profile of the combination therapy. Accordingly, in certain embodiments of the methods of the invention, the first treatment cycle comprises administering a first dose of the anti-androgen compound at least 3 days after administering a first therapeutic dose of the PSMA-targeted T-cell engaging molecule. In some embodiments, the first cycle comprises administering a first dose of the anti-androgen compound about 3 days to about 14 days, about 4 days to about 10 days, or about 5 days to about 9 days after administering the first therapeutic dose of the PSMA-targeted T-cell engaging molecule. The delay period between the administration of the first therapeutic dose of the PSMA-targeted T-cell engaging molecule and the administration of the first dose of the anti-androgen compound in the first cycle can be about 3 days, about 4 days, about 5 days, about 6 days, about 7 days, about 8 days, about 9 days, about 10 days, about 11 days, about 12 days, about 13 days, or about 14 days. In one embodiment, the first cycle comprises administering the first dose of the anti-androgen compound about 5 days after administering the first therapeutic dose of the PSMA-targeted T-cell engaging molecule. In another embodiment, the first cycle comprises administering the first dose of the anti-androgen compound about 7 days after administering the first therapeutic dose of the PSMA-targeted T-cell engaging molecule.

The first cycle or initiation cycle is typically administered to a patient when the patient begins a course of treatment with a PSMA-targeted T-cell engaging molecule. The first cycle regimen described herein can also be administered to a patient when the patient re-starts a course of treatment with a PSMA-targeted T-cell engaging molecule, for example, following a treatment-free period, dosing interruption (e.g. when a patient didn't complete a previous treatment cycle), or a relapse or progression of a cancer in the patient. The first cycle regimen can also be administered to a patient if the patient switches to a course of treatment with a different PSMA-targeted T-cell engaging molecule or different anti-androgen compound. In some embodiments of the methods of the invention, the duration of the first cycle or initiation cycle is from about 14 days to about 56 days, for example, from about 14 days to about 28 days, from about 21 days to about 42 days, from about 28 days to about 49 days, or from about 21 days to about 28 days. In certain embodiments, the duration of the first cycle or initiation cycle is about 28 days.

In one aspect, the methods of the invention comprise administering to a patient a therapeutically effective dose of a PSMA-targeted T-cell engaging molecule and a therapeutically effective dose of an anti-androgen compound. A “therapeutically effective dose” or “therapeutic dose” refers to an amount of the compound sufficient to treat or ameliorate prostate cancer or one or more of its symptoms, particularly a state or symptoms associated with prostate cancer, or otherwise prevent, hinder, retard or reverse the progression of prostate cancer or any other undesirable symptom associated with prostate cancer in any way whatsoever. The amounts of the therapeutic dose may vary depending on the characteristics of the patient to be treated, the route of administration, the type, grade or stage of cancer diagnosed in the patient, and the specific PSMA-targeted T-cell engaging molecule and anti-androgen compound administered to the patient. The therapeutic doses for the PSMA-targeted T-cell engaging molecule and/or the anti-androgen compound administered to the patient in the combination therapy methods of the invention may be different than the therapeutic doses of the same compounds employed in monotherapy methods. For instance, the therapeutic dose of one or both compounds may be less than the therapeutic dose of the compounds administered as part of a monotherapy due to, e.g., a synergistic anti-tumor effect. Specific therapeutic doses for PSMA-targeted T-cell engaging molecules and anti-androgen compounds can be determined from dose-exploration human clinical trials, such as those described in the Examples, or may in some cases be estimated from relevant animal models for the particular cancer to be treated. Suitable dosages for specific PSMA-targeted T-cell engaging molecules and anti-androgen compounds are described in more detail herein.

In some embodiments of the methods of the invention, the first dose of the PSMA-targeted T-cell engaging molecule and/or the anti-androgen compound administered to the patient, e.g., in the first cycle, is a therapeutic dose. In other embodiments, the first dose of the PSMA-targeted T-cell engaging molecule and/or the anti-androgen compound administered to the patient, e.g., in the first cycle, is not the therapeutic dose. For instance, the first dose of the PSMA-targeted T-cell engaging molecule and/or the anti-androgen compound administered to the patient may be a dose that is lower than a therapeutic dose (e.g. a titration or priming dose). Such priming doses, particularly for PSMA-targeted T-cell engaging molecules, may be employed to reduce the number or severity of adverse events, such as CRS events, when the patient is first exposed to the PSMA-targeted T-cell engaging molecule. As used herein, the term “priming dose” refers to a dose or amount of a PSMA-targeted T-cell engaging molecule that primes a patient for subsequent administration of a therapeutic dose of the PSMA-targeted T-cell engaging molecule such that administration of the therapeutic dose produces fewer or less severe adverse events, e.g. fewer or less severe CRS events, in the patient. In some embodiments, the priming dose may be lower than a therapeutic dose but is a dose that is sufficient to prime a patient's T-cells, e.g. to release cytokines, such that administration of a subsequent greater dose or therapeutic dose of the T-cell engaging molecule produces an attenuated increase in cytokine secretion. In certain embodiments, the priming dose is sufficient to increase the proportion of activated peripheral T-cells in the patient (e.g. increases the proportion of CD69+CD8+ peripheral T-cells) relative to the proportion of activated T-cells in the patient prior to receiving the dose of the T-cell engaging molecule. The priming dose may be a fraction of the therapeutic dose, such as from about 10% to about 80% of the therapeutic dose, from about 20% to about 75%, from about 15% to about 50%, from about 25% to about 70%, or from about 30% to about 60% of the therapeutic dose. In one embodiment, the priming dose of the PSMA-targeted T-cell engaging molecule is about 30% of the therapeutic dose. In another embodiment, the priming dose of the PSMA-targeted T-cell engaging molecule is about 60% of the therapeutic dose. One or more priming doses of the PSMA-targeted T-cell engaging molecule may be administered to the patient before administration of the therapeutic dose. In certain embodiments of the methods of the invention, the first dose of the anti-androgen compound administered to the patient in the first cycle is a therapeutic dose and the first dose of the PSMA-targeted T-cell engaging molecule administered to the patient in the first cycle is a priming dose. In certain other embodiments of the methods of the invention, the first dose of the anti-androgen compound administered to the patient in the first cycle is a therapeutic dose and the first dose of the PSMA-targeted T-cell engaging molecule administered to the patient in the first cycle is a therapeutic dose.

In certain embodiments of the methods of the invention, the first cycle further comprises administering one or more priming doses of the PSMA-targeted T-cell engaging molecule to the patient prior to administration of the first therapeutic dose of the T-cell engaging molecule. In such embodiments, the first therapeutic dose of the T-cell engaging molecule can be administered about 1 day to about 21 days, about 1 day to about 14 days, or about 1 day to about 7 days after administration of the first priming dose of the PSMA-targeted T-cell engaging molecule to the patient. In certain embodiments, the first therapeutic dose of the PSMA-targeted T-cell engaging molecule is administered to the patient about 3 days, about 4 days, about 5 days, about 6 days, or about 7 days after administration of the first priming dose of the PSMA-targeted T-cell engaging molecule. In embodiments in which two or more priming doses of the PSMA-targeted T-cell engaging molecule are administered to the patient, the priming doses can be the same or they may be different. For example, in some such embodiments, the priming dose of the PSMA-targeted T-cell engaging molecule may increase at one or more subsequent dosing intervals as a series of increasing dose steps. Such a step dosing regimen can be employed in embodiments in which two or more priming doses are administered prior to administration of the therapeutic dose during the first cycle and may comprise one or more dosage steps (e.g. one or more dose increases).

An anti-androgen compound administered according to the methods of the invention refers to a compound that inhibits or blocks the biological activity of androgens in one or more cells or tissues of the body. In some embodiments, the anti-androgen compound inhibits or blocks the synthesis of androgen, for example, by inhibiting the function of the CYP17A1 enzyme. Exemplary androgen biosynthesis inhibitors include abiraterone and ketoconazole. In other embodiments, the anti-androgen compound is an androgen receptor antagonist, such as flutamide, bicalutamide, nilutamide, enzalutamide, apalutamide, or darolutamide. In certain embodiments, the anti-androgen compound used in the methods of the invention is abiraterone, abiraterone acetate, ketoconazole, flutamide, bicalutamide, nilutamide, enzalutamide, apalutamide, or darolutamide. In other embodiments, the anti-androgen compound is enzalutamide, abiraterone, abiraterone acetate, apalutamide, or darolutamide. In one particular embodiment, the anti-androgen compound used in the methods of the invention is enzalutamide. In another particular embodiment, the anti-androgen compound used in the methods of the invention is abiraterone or abiraterone acetate.

In certain embodiments of the methods of the invention, the anti-androgen compound is administered orally. Suitable therapeutic oral doses for the anti-androgen compounds can be from about 50 mg to about 1,000, from about 150 mg to about 800 mg, or from about 200 mg to about 600 mg, depending on the specific anti-androgen compound employed. Such doses can be administered once per day, twice per day, or three times per day for the duration of the treatment cycle. In certain embodiments, the anti-androgen compound is administered to the patient orally once per day (QD) In some embodiments, the oral doses are in the form of tablets or capsules. In some such embodiments, each tablet or capsule may contain unit doses less than the total therapeutic dose to be administered such that more than one tablet or capsule is administered to the patient at each specified dosing interval.

In some embodiments, the anti-androgen compound is enzalutamide, and the therapeutic oral dose is from about 40 mg to about 160 mg administered once per day (QD). In one particular embodiment of the methods of the invention, enzalutamide is administered to the patient orally at a dose of 160 mg (e.g., four 40 mg tablets or capsules or two 80 mg tablets) once per day. In other embodiments, the anti-androgen compound is abiraterone or abiraterone acetate, and the therapeutic oral dose is from about 250 mg to about 1,000 mg or from about 500 mg to about 1,000 mg administered once per day. In one such embodiment, abiraterone or abiraterone acetate is administered to the patient orally at a dose of 1,000 mg (e.g., four 250 mg tablets or two 500 mg tablets) once per day. In embodiments in which the patient is administered abiraterone or abiraterone acetate, a 5 mg dose of prednisone may be orally administered twice per day (BID). In other embodiments in which the patient is administered abiraterone or abiraterone acetate, a 10 mg dose of prednisolone may be orally administered once per day. In certain other embodiments of the methods of the invention, the anti-androgen compound is darolutamide, and the therapeutic oral dose is from about 300 mg to about 600 mg administered twice per day (BID). In one such embodiment, darolutamide is administered to the patient orally at a dose of 600 mg (e.g., two 300 mg tablets) twice per day. In other embodiments, the anti-androgen compound is apalutamide, and the therapeutic oral dose is from about 120 mg to about 240 mg administered once per day (QD). In one such embodiment, apalutamide is administered to the patient orally at a dose of 240 mg (e.g., four 60 mg tablets) once per day.

The methods described herein comprise administering to a patient a PSMA-targeted T-cell engaging molecule, which refers to a T-cell engaging molecule that specifically binds to PSMA and CD3. The term “T-cell engaging molecule” refers to a molecule that comprises at least one domain in which the structure is derived from or comprises the minimum structural features of an antibody, e.g., of a full-length immunoglobulin molecule, that allow for specific binding to an antigen on the surface of a T cell, such as CD3. Thus, a T-cell engaging molecule according to the invention generally comprises one or more binding domains, each of which will typically comprise the minimum structural requirements of an antibody that allow for specific target binding. This minimum requirement may, for example, be defined by the presence of at least three light chain “complementarity determining regions” or CDRs (i.e. CDRL1, CDRL2 and CDRL3 of a VL region) and/or three heavy chain CDRs (i.e. CDRH1, CDRH2 and CDRH3 of a VH region), and preferably all six CDRs from both the light and heavy chain variable regions. The T-cell engaging molecules according to the invention may comprise domains or regions (e.g. CDRs or variable regions) from monoclonal, chimeric, humanized and human antibodies.

Preferably, the T-cell engaging molecules used in the methods of the invention are proteins and comprise one or more polypeptide chains. A polypeptide, as used herein, refers to a polymer of amino acids comprising at least 50 amino acids, preferably at least 100 amino acids. In some embodiments, the T-cell engaging molecules administered according to the methods of the invention are single-chain polypeptides. In other embodiments, the T-cell engaging molecules administered according to the methods of the invention comprise two or more polypeptide chains—e.g. are polypeptide dimers or multimers. In certain embodiments, the T-cell engaging molecules administered according to the methods of the invention comprise four polypeptide chains, and may, e.g. have the format of an antibody or an immunoglobulin protein. In certain other embodiments, the T-cell engaging molecules administered according to the methods of the invention comprise three polypeptide chains.

As used herein, the term “antibody” generally refers to a tetrameric immunoglobulin protein comprising two light chain polypeptides (about 25 kDa each) and two heavy chain polypeptides (about 50-70 kDa each). The term “light chain” or “immunoglobulin light chain” refers to a polypeptide comprising, from amino terminus to carboxyl terminus, a single immunoglobulin light chain variable region (VL) and a single immunoglobulin light chain constant domain (CL). The immunoglobulin light chain constant domain (CL) can be a human kappa (κ) or human lambda (λ) constant domain. The term “heavy chain” or “immunoglobulin heavy chain” refers to a polypeptide comprising, from amino terminus to carboxyl terminus, a single immunoglobulin heavy chain variable region (VH), an immunoglobulin heavy chain constant domain 1 (CH1), an immunoglobulin hinge region, an immunoglobulin heavy chain constant domain 2 (CH2), an immunoglobulin heavy chain constant domain 3 (CH3), and optionally an immunoglobulin heavy chain constant domain 4 (CH4). Heavy chains are classified as mu (μ), delta (Δ), gamma (γ), alpha (α), and epsilon (ε), and define the antibody's isotype as IgM, IgD, IgG, IgA, and IgE, respectively. The IgG-class and IgA-class antibodies are further divided into subclasses, namely, IgG1, IgG2, IgG3, and IgG4, and IgA1 and IgA2, respectively. The heavy chains in IgG, IgA, and IgD antibodies have three constant domains (CH1, CH2, and CH3), whereas the heavy chains in IgM and IgE antibodies have four constant domains (CHI, CH2, CH3, and CH4). The immunoglobulin heavy chain constant domains can be from any immunoglobulin isotype, including subtypes. The antibody chains are linked together via inter-polypeptide disulfide bonds between the CL domain and the CHI domain (i.e. between the light and heavy chain) and between the hinge regions of the two antibody heavy chains.

Variable regions of immunoglobulin chains generally exhibit the same overall structure, comprising relatively conserved framework regions (FR) joined by three hypervariable regions, more often called “complementarity determining regions” or CDRs. The CDRs from the two chains of each heavy chain and light chain pair typically are aligned by the framework regions to form a structure that binds specifically to a specific epitope on the target protein (e.g., PSMA or CD3). From N-terminus to C-terminus, naturally-occurring light and heavy chain variable regions both typically conform with the following order of these elements: FRI, CDR1, FR2, CDR2, FR3, CDR3, and FR4. A numbering system has been devised for assigning numbers to amino acids that occupy positions in each of these domains. This numbering system is defined in Kabat Sequences of Proteins of Immunological Interest (1987 and 1991, NIH, Bethesda, MD), or Chothia & Lesk, 1987, J. Mol. Biol. 196:901-917; Chothia et al., 1989, Nature 342:878-883. The CDRs and FRs of a given antibody may be identified using this system. Other numbering systems for the amino acids in immunoglobulin chains include IMGT® (the international ImMunoGeneTics information system; Lefranc et al., Dev. Comp Immunol. 29:185-203; 2005) and AHo (Honegger and Pluckthun, J. Mol. Biol. 309 (3): 657-670; 2001).

The T-cell engaging molecules used in the methods of the invention are preferably at least bispecific T-cell engaging molecules. The term “bispecific T-cell engaging molecule” refers to a molecule capable of specifically binding to two different antigens. In the context of the present invention, such bispecific T-cell engaging molecules specifically bind to PSMA (e.g. human PSMA) on the cell surface of target cells and CD3 (e.g. human CD3) on the cell surface of T cells. The term “PSMA-targeted T-cell engaging molecule” is used herein to refer to a T-cell engaging molecule that specifically binds to PSMA and CD3. A T-cell engaging molecule or binding domain thereof “specifically binds” to a target antigen when it has a significantly higher binding affinity for, and consequently is capable of distinguishing, that antigen compared to its affinity for other unrelated proteins, under similar binding assay conditions. T-cell engaging molecules or binding domains thereof that specifically bind an antigen may bind to that antigen with an equilibrium dissociation constant (K_{D})≤1×10⁻⁶ M. T-cell engaging molecules or binding domains thereof specifically bind antigen with “high affinity” when the K_{D} is ≤1×10⁻⁸ M. In one embodiment, the T-cell engaging molecules or binding domains thereof used in the methods of the invention bind to human PSMA and/or human CD3 with a K_{D} of ≤5×10⁻⁷ M. In another embodiment, the T-cell engaging molecules or binding domains thereof used in the methods of the invention bind to human PSMA and/or human CD3 with a K_{D} of ≤1×10⁻⁷M. In yet another embodiment, the T-cell engaging molecules or binding domains thereof used in the methods of the invention bind to human PSMA and/or human CD3 with a K_{D} of ≤5×10⁻⁸ M. In another embodiment, the T-cell engaging molecules or binding domains thereof used in the methods of the invention bind to human PSMA and/or human CD3 with a K_{D} of ≤2×10⁻⁸ M. In certain embodiments, the T-cell engaging molecules or binding domains thereof used in the methods of the invention bind to human PSMA and/or human CD3 with a K_{D} of ≤1×10⁻⁸ M. In other embodiments, the T-cell engaging molecules or binding domains thereof used in the methods of the invention bind to human PSMA and/or human CD3 with a K_{D} of ≤1×10⁻⁹ M.

Affinity is determined using a variety of techniques, an example of which is an affinity ELISA assay. In various embodiments, affinity is determined by a surface plasmon resonance assay (e.g., BIAcore®-based assay). Using this methodology, the association rate constant (kₐ in M⁻¹s⁻¹) and the dissociation rate constant (k_{d} in s⁻¹) can be measured. The equilibrium dissociation constant (K_{D} in M) can then be calculated from the ratio of the kinetic rate constants (k_{d}/kₐ). In some embodiments, affinity is determined by a kinetic method, such as a Kinetic Exclusion Assay (KinExA) as described in Rathanaswami et al. Analytical Biochemistry, Vol. 373:52-60, 2008. Using a KinExA assay, the equilibrium dissociation constant (K_{D} in M) and the association rate constant (kₐ in M⁻¹s⁻¹) can be measured. The dissociation rate constant (k_{d} in s⁻¹) can be calculated from these values (K_{D}×kₐ). In other embodiments, affinity is determined by a bio-layer interferometry method, such as that described in Kumaraswamy et al., Methods Mol. Biol., Vol. 1278:165-82, 2015 and employed in Octet® systems (Pall ForteBio). The kinetic (kₐ and k_{d}) and affinity (K_{D}) constants can be calculated in real-time using the bio-layer interferometry method. In some embodiments, the T-cell engaging molecules or binding domains thereof described herein exhibit desirable characteristics such as binding avidity as measured by kₐ (dissociation rate constant) for human PSMA and/or human CD3 of about 10⁻², 10⁻³, 10⁻⁴, 10⁻⁵, 10⁻⁶, 10⁻⁷, 10⁻⁸, 10⁻⁹, 10⁻¹⁰ s⁻¹ or lower (lower values indicating higher binding avidity), and/or binding affinity as measured by K_{D} (equilibrium dissociation constant) for human PSMA and/or human CD3 of about 10⁻⁷, 10⁻⁸, 10⁻⁹, 10⁻¹⁰, 10⁻¹¹ M or lower (lower values indicating higher binding affinity).

In some embodiments, PSMA-targeted T-cell engaging molecules used in the methods of the invention may be antibodies and have the general structure of a full-length immunoglobulin. For example, the PSMA-targeted T-cell engaging molecules may comprise two full-length antibody heavy chains and two full-length antibody light chains. In particular embodiments, the T-cell engaging molecules are heterodimeric antibodies (used interchangeably herein with “hetero immunoglobulins” or “hetero Igs”), which refer to antibodies comprising two different light chains and two different heavy chains. For instance, in some embodiments, the heterodimeric antibody comprises a light chain and heavy chain from an anti-PSMA antibody and a light chain and heavy chain from an anti-CD3 antibody.

The PSMA-targeted T-cell engaging molecules employed in the methods of the invention may also comprise fragments of full-length antibodies, such as VH, VHH, VL, (s)dAb, Fv, light chain (VL-CL), Fd (VH-CH1), heavy chain, Fab, Fab′, F(ab′)₂ or “r IgG” (“half antibody” consisting of a heavy chain and a light chain). PSMA-targeted T-cell engaging molecules according to the invention may also comprise modified fragments of antibodies. Examples of such modified fragments include, but are not limited to, single-chain variable fragment (scFv), di-scFv or bi(s)-scFv, scFv-Fc, scFv-zipper, single-chain Fab (scFab), Fab₂, Fab₃, diabodies, single-chain diabodies, tandem diabodies (Tandabs), tandem di-scFv, tandem tri-scFv, “minibodies” exemplified by a structure which is as follows: (VH-VL-CH3)₂, (scFv-CH3)₂, ((scFv)₂-CH3+CH3), ((scFv)₂-CH3) or (scFv-CH3-scFv)₂, multibodies, such as triabodies or tetrabodies, and single domain antibodies, such as nanobodies or single variable domain antibodies comprising merely one variable region, which might be VHH, VH or VL, that specifically binds to an antigen or target independently of other variable regions or domains. In some embodiments, PSMA-targeted T-cell engaging molecules used in the methods of the invention comprise a binding domain derived from a single variable domain antibody that comprises only a heavy chain variable region. In some such embodiments, the binding domain comprising the heavy chain variable region can be fused to one or more constant regions from a an immunoglobulin heavy chain, such as a CH2, a CH3, or an Fc domain.

In certain embodiments, the PSMA-targeted T-cell engaging molecules used in the methods of the invention are multivalent. The valency of the T-cell engaging molecule denotes the number of individual antigen-binding domains within the T-cell engaging molecule. For example, the terms “monovalent,” “bivalent,” and “tetravalent” with reference to the T-cell engaging molecules in the context of the invention refer to T-cell engaging molecules with one, two, and four antigen-binding domains, respectively. Thus, a multivalent T-cell engaging molecule comprises two or more antigen-binding domains. A T-cell engaging molecule can have more antigen-binding domains (e.g. a higher valency) than specificities. For example, a T-cell engaging molecule having two antigen-binding domains for a first target (e.g. PSMA) and one antigen-binding domain for a second target (CD3)—or vice versa—is considered to be trivalent (three antigen-binding domains) and bispecific (binds to two antigens). In certain embodiments, the PSMA-targeted T-cell engaging molecules used in the methods of the invention are bivalent. Thus, such bispecific, bivalent T-cell engaging molecules contain two antigen binding domains: one antigen-binding domain for PSMA (e.g. human PSMA) and one antigen-binding domain for CD3 (e.g. human CD3).

Exemplary PSMA-targeted T-cell engaging molecules for use in the methods of the invention are described in more detail herein. Other suitable PSMA-targeted T-cell engaging molecules that can be administered in the combination therapy methods of the invention are known in the art, such as those described in WO 2010/037836, WO 2011/121110, WO 2012/145714, WO 2017/023761, WO 2017/121905, WO 2017/134158, WO 2018/098356, WO 2019/224718, WO 2020/206330, and WO 2021/231976, all of which are hereby incorporated by reference in their entireties Exemplary ranges of therapeutic doses of a PSMA-targeted T-cell engaging molecule that may be administered according to the methods of the invention may include, but are not limited to, doses of about 50 μg to about 200 mg, from about 200 μg to about 80 mg, from about 90 μg to about 30 mg, from about 300 μg to about 15 mg, from about 150 μg to about 2 mg, from about 6 mg to about 25 mg, from about 1 mg to about 20 mg, from about 10 mg to about 100 mg, or from about 50 mg to about 150 mg. Depending on the specific PSMA-targeted T-cell engaging molecule employed and its half-life, the therapeutic dose of the PSMA-targeted T-cell engaging molecule may be administered at a dosing interval of once per week (QW), once every two weeks (Q2W), once every three weeks (Q3W), or once per month (QM). In certain embodiments, the therapeutic dose of the PSMA-targeted T-cell engaging molecule is administered once every 7 days or once per week. In certain other embodiments, the therapeutic dose of the PSMA-targeted T-cell engaging molecule is administered once every 14 days or once every two weeks.

In some embodiments of the methods described herein, the PSMA-targeted T-cell engaging molecule is administered to the patient parenterally. Parenteral administration refers to administration of the molecule by routes other than through the gastrointestinal tract and can include intraperitoneal, intramuscular, intravenous, intraarterial, intradermal, subcutaneous, intracerebral, intracerebroventricular, and intrathecal administration. In certain embodiments of the methods of the invention, the PSMA-targeted T-cell engaging molecule is administered to the patient intravenously. In other embodiments of the methods of the invention, the PSMA-targeted T-cell engaging molecule is administered to the patient subcutaneously.

Parenteral or intravenous administration can be performed by injection (e.g., using a needle and a syringe) or by infusion (e.g., via a catheter and a pump system). It is envisaged that the administration of the PSMA-targeted T-cell engaging molecule according to some embodiments of the methods of the present invention is via intravenous injection or via intravenous infusion. Usually, an intravenous (IV) infusion is administered via a line, a port or a catheter (small, flexible tube), such as a central venous access or a central venous catheter (CVC), which is a catheter placed into a large vein, or a peripheral venous catheter (PVC), which is a catheter placed into a peripheral vein. In general, catheters or lines can be placed in veins in the neck (internal jugular vein), chest (subclavian vein or axillary vein), groin (femoral vein), or through veins in the arms (also known as a PICC line, or peripherally inserted central catheters). Central IV lines have catheters that are advanced through a vein and empty into a large central vein, usually the superior vena cava, inferior vena cava or even the right atrium of the heart. A peripheral intravenous (PIV) line is used on peripheral veins (the veins in the arms, hands, legs and feet). A port is a central venous line that does not have an external connector; instead, it has a small reservoir that is covered with silicone rubber and is implanted under the skin. Medication is administered intermittently by placing a small needle through the skin, piercing the silicone, into the reservoir. When the needle is withdrawn, the reservoir cover reseals itself. The cover can accept hundreds of needle sticks during its lifetime.

In certain embodiments of the methods of the invention, one or more doses of the PSMA-targeted T-cell engaging molecule is administered to the patient by a continuous intravenous infusion over an extended period of time. As used herein, a continuous intravenous infusion refers to a controlled method of intravenous administration of the PSMA-targeted T-cell engaging molecule given over a period of time longer than about 3 hours, more typically longer than about 6 hours, without interruption or without substantial interruption. The continuous intravenous infusion may be administered by way of a fluid delivery device or small pump system including a fluid driving mechanism for driving fluid out of a reservoir and an actuating mechanism for actuating the driving mechanism. Pump systems for such administration may include a needle or a cannula for penetrating the skin of a patient and delivering the infusion solution into the patient's body. The pump system can be connected to the patient for 24 hours up to several days. Pump systems for delivering intravenous infusions are known in the art. Depending on the duration of the continuous infusion, the bags or reservoirs containing the infusion solution in the pump system may need to be exchanged or replaced. During the exchange of the bag or reservoir in the pump system, a temporary interruption of the otherwise uninterrupted flow of the infusate may occur. Such temporary interruptions occurring from bag or reservoir replacement do not constitute an interruption or substantial interruption of the intravenous administration, and the period of time during which the bag or reservoir is replaced would still be considered to be within the period of a continuous intravenous infusion as the term is used herein.

In other embodiments of the methods of the invention, one or more doses of the PSMA-targeted T-cell engaging molecule is administered by a bolus intravenous infusion. As used herein, a bolus intravenous infusion, used interchangeably herein with short intravenous infusion, refers to an intravenous infusion of a small volume (e.g., 20 mL to 100 mL) administered over a period of, at most three hours, and more typically over a period of about 30 min to about 90 min. In some embodiments of the methods of the invention, a bolus intravenous infusion is an intravenous infusion administered over about 30 min to about 60 min. In certain embodiments of the methods of the invention, a bolus intravenous infusion is an intravenous infusion administered over about 60 min (e.g., 55 min to 65 min).

A combination of administration routes may be used to administer the PSMA-targeted T-cell engaging molecule according to the methods of the invention. For example, in some embodiments, the first dose (e.g., a priming dose) of the PSMA-targeted T-cell engaging molecule, particularly in the first cycle, may be administered by continuous intravenous infusion over an extended period of time, such as over I day to 7 days, followed by administration of subsequent doses (e.g., therapeutic doses) by a bolus intravenous infusion or subcutaneous injection. Thus, in some embodiments of the methods of the invention, the first dose (e.g., a first priming dose) of the PSMA-targeted T-cell engaging molecule in the first cycle is administered by continuous intravenous infusion over a period of at least 24 hours, for example over a period of 1 to 14 days, 1 to 7 days, or 1 to 5 days. In particular embodiments, the first dose (e.g., a first priming dose) of the PSMA-targeted T-cell engaging molecule in the first cycle is administered by continuous intravenous infusion over a period of about 3 days (i.e., a 72-hour infusion). In these and other embodiments, the continuous intravenous infusion is given at a constant flow rate—that is the continuous intravenous infusion delivers the dose of the PSMA-targeted T-cell engaging molecule at a constant rate over the period of the infusion. By way of example, for a dose of 3 mg, a continuous intravenous infusion at a constant flow rate given over 3 days would deliver the dose at a rate of 1 mg per day such that the total dose of 3 mg would be delivered at the completion of the 3-day infusion period. Alternatively, in some embodiments, the continuous intravenous infusion may be given at a variable flow rate such that the dose is delivered at different doses per day over the period of infusion. For instance, in one such embodiment, the flow rate of the continuous infusion can be adjusted such that increasing doses are given each day over the infusion period to deliver the total dose at the completion of the infusion period.

In some embodiments of the methods of the invention, the first cycle comprises administering a dose of the PSMA-targeted T-cell engaging molecule at a dosing interval of once every 7 days (or once per week) and administering a dose of an anti-androgen compound at a dosing interval of once per day, wherein the first dose of the anti-androgen compound is administered at least 3 days after administering the first therapeutic dose of the PSMA-targeted T-cell engaging molecule. An exemplary dosing schedule according to such embodiments in which the duration of the first cycle is 28 days comprises administration of a dose of the PSMA-targeted T-cell engaging molecule on each of days 1, 8, 15, and 22 of the first cycle (e.g., by bolus intravenous infusion) and administration of a dose of the anti-androgen compound once per day on each of days 15 to 28 of the first cycle (e.g., by oral administration). In other embodiments of the methods of the invention, the first cycle comprises administering a dose of the PSMA-targeted T-cell engaging molecule at a dosing interval of once every 14 days (or once every two weeks) and administering a dose of an anti-androgen compound at a dosing interval of once per day, wherein the first dose of the anti-androgen compound is administered at least 3 days after administering the first therapeutic dose of the PSMA-targeted T-cell engaging molecule. An exemplary dosing schedule according to these embodiments in which the duration of the first cycle is 28 days comprises administration of a dose of the PSMA-targeted T-cell engaging molecule on each of days 1 and 15 of the first cycle (e.g., by bolus intravenous infusion) and administration of a dose of the anti-androgen compound once per day on each of days 15 to 28 of the first cycle (e.g., by oral administration). In any of the foregoing embodiments, all of the doses of the PSMA-targeted T-cell engaging molecule administered during the first cycle may be the same and be therapeutic doses. In alternative embodiments, the first dose of the PSMA-targeted T-cell engaging molecule (e.g. dose given on day 1 of the cycle) may be different than all subsequent doses of the T-cell engaging molecule administered during the first cycle. For example, the first dose of the PSMA-targeted T-cell engaging molecule may be a priming dose as described herein and all subsequent doses of the PSMA-targeted T-cell engaging molecule may be therapeutic doses.

In certain embodiments of the methods of the invention, the first cycle comprises administering a priming dose of the PSMA-targeted T-cell engaging molecule by continuous intravenous infusion over about 3 days, subsequently administering a therapeutic dose of the PSMA-targeted T-cell engaging molecule by bolus intravenous infusion on a dosing interval of 7 days or 14 days, and administering a dose of an anti-androgen compound at a dosing interval of once per day, wherein the first dose of the anti-androgen compound is administered at least 3 days after administering the first therapeutic dose of the PSMA-targeted T-cell engaging molecule. In embodiments in which the first cycle has a duration of 28 days, an exemplary dosing schedule may comprise administration of a priming dose (which can be any of the priming doses described herein) of the PSMA-targeted T-cell engaging molecule by continuous intravenous infusion over days 1 to 3 of the first cycle, administration of a therapeutic dose of the PSMA-targeted T-cell engaging molecule by bolus intravenous infusion on days 8 and 22 of the first cycle, and oral administration of a dose of the anti-androgen compound once per day on each of days 15 to 28 of the first cycle.

In some embodiments, the methods of the invention further comprise administering to the patient at least one maintenance cycle of the anti-androgen compound in combination with the PSMA-targeted T-cell engaging molecule after administration of the first cycle or initiation cycle. As used herein, a “maintenance cycle” is a treatment cycle in which the PSMA-targeted T-cell engaging molecule and the anti-androgen compound are administered at a dose and dosing frequency designed to maintain a threshold level of exposure of both compounds at therapeutic levels in the patient. In certain embodiments, the dosing frequency employed in the maintenance cycle for the PSMA-targeted T-cell engaging molecule, the anti-androgen compound, or both compounds is lower than the dosing frequency employed in the first cycle (i.e. the dosing interval in the maintenance cycle is longer than the dosing interval in the first cycle). In some embodiments, the maintenance cycle is administered immediately after the completion of the first cycle. Accordingly, in such embodiments, there are no treatment-free periods or breaks between the end of the first cycle or initiation cycle and the start of the maintenance cycle. In one such embodiment, the maintenance cycle is administered the following day after completing the first cycle or initiation cycle. In other embodiments, there is a treatment-free period or break between the completion of the first cycle or initiation cycle and the administration of the maintenance cycle. Preferably, the treatment-free period between the first cycle or initiation cycle and the maintenance cycle is no longer than the dosing interval for the PSMA-targeted T-cell engaging molecule employed in the maintenance cycle. In one embodiment, the maintenance cycle is administered about 7 days following completion of the first cycle or initiation cycle. In another embodiment, the maintenance cycle is administered about 14 days following completion of the first cycle or initiation cycle.

Multiple maintenance cycles (e.g., 2, 3, 4, 5, 6, 7, 8, 9, 10, 11, 12 or more cycles) can be administered to the patient depending on the desired duration of treatment for that patient. For instance, the patient may receive maintenance cycles of the PSMA-targeted T-cell engaging molecule in combination with the anti-androgen compound until the patient achieves a desired level of response, such as a complete response or partial response. In some embodiments, two or more maintenance cycles are administered to the patient. In other embodiments, four or more maintenance cycles are administered to the patient. In still other embodiments, six to twelve maintenance cycles are administered to the patient. In certain embodiments, the maintenance cycles are administered consecutively with no treatment-free periods between the maintenance cycles. If a treatment interruption is necessary, ideally the duration of the treatment-free period will be no greater than twice the dosing interval for the PSMA-targeted T-cell engaging molecule employed in the maintenance cycle. By way of example, if the dosing interval for the PSMA-targeted T-cell engaging molecule employed in the maintenance cycle is once every 14 days (e.g., once every two weeks), the treatment-free period between maintenance cycles will preferably be about 28 days or less.

In certain embodiments of the methods of the invention, the maintenance cycle comprises administering a therapeutic dose of the PSMA-targeted T-cell engaging molecule (such as any of the therapeutic doses described herein) at a dosing interval of at least 7 days and administering the anti-androgen compound orally once per day on each day of the cycle. For instance, in some embodiments of the methods of the invention, the maintenance cycle comprises administering a therapeutic dose of the PSMA-targeted T-cell engaging molecule by a bolus intravenous infusion or subcutaneous injection once every 7 days (e.g., weekly, QW dosing) and administering the anti-androgen compound orally once per day on each day of the cycle. In other embodiments of the methods of the invention, the maintenance cycle comprises administering a therapeutic dose of the PSMA-targeted T-cell engaging molecule by a bolus intravenous infusion or subcutaneous injection once every 14 days (e.g., once every two weeks, Q2W dosing) and administering the anti-androgen compound orally once per day on each day of the cycle. In still other embodiments, the therapeutic dose of the PSMA-targeted T-cell engaging molecule may be administered by a bolus intravenous infusion or subcutaneous injection at longer dosing intervals during the maintenance cycle, such as once every three weeks or once every four weeks. In such embodiments, the anti-androgen compound may continue to be administered orally once per day on each day of the cycle. Preferably, the therapeutic dose of the PSMA-targeted T-cell engaging molecule administered during the maintenance cycle is the same at each dosing interval, e.g., each weekly or biweekly dosing interval (e.g., a fixed dose for the entire maintenance cycle). In these and other embodiments, the therapeutic dose and dosing frequency of the PSMA-targeted T-cell engaging molecule administered during the maintenance cycle is the same from one maintenance cycle to the next maintenance cycle.

According to some embodiments of the methods of the invention, the duration of the maintenance cycle is from about 14 days to about 60 days, for example, from about 14 days to about 28 days, from about 21 days to about 42 days, from about 28 days to about 49 days, from about 28 days to about 56 days, or from about 21 days to about 28 days. In certain embodiments, the duration of the maintenance cycle is about 28 days. In some such embodiments, a therapeutic dose of the PSMA-targeted T-cell engaging molecule is administered by bolus intravenous infusion on days 1 and 15 of each maintenance cycle and the anti-androgen compound is administered orally once per day on each day of the maintenance cycle (i.e., each of days 1 to 28). In other embodiments in which the duration of the maintenance cycle is about 28 days, a therapeutic dose of the PSMA-targeted T-cell engaging molecule is administered by bolus intravenous infusion on days 1, 8, 15, and 22 of each maintenance cycle and the anti-androgen compound is administered orally once per day on each day of the maintenance cycle (i.e., each of days 1 to 28).

In some embodiments, the PSMA-targeted T-cell engaging molecules employed in the methods of the invention comprise a first binding domain that specifically binds to PSMA (e.g. human PSMA) and a second binding domain that specifically binds to CD3 (e.g. human CD3). As used herein, the term “antigen-binding domain,” which is used interchangeably with “binding domain,” refers to the region of the T-cell engaging molecule that contains the amino acid residues that interact with the antigen and confer on the T-cell engaging molecule its specificity and affinity for the antigen. In certain embodiments, one or more binding domains of the T-cell engaging molecules may be derived from an antibody or antigen-binding fragment thereof. For instance, the binding domains of the PSMA-targeted T-cell engaging molecules used in the methods of the invention may comprise one or more CDRs from the light and heavy chain variable regions of antibodies that specifically bind to human PSMA and/or human CD3. In some embodiments, the anti-PSMA binding domain of the T-cell engaging molecules comprises all six CDRs of the heavy and light chain variable regions of an anti-PSMA antibody described herein or known in the art and the anti-CD3 binding domain of the T-cell engaging molecules comprises all six CDRs of the heavy and light chain variable regions of an anti-CD3 antibody described herein or known in the art. In some embodiments, the binding domains (the anti-PSMA binding domain, the anti-CD3 binding domain or both) of the PSMA-targeted T-cell engaging molecules used in the methods of the invention comprise a Fab, a Fab′, a F(ab′)₂, a Fv, a single-chain variable fragment (scFv), or a nanobody. In one embodiment, both binding domains of the PSMA-targeted T-cell engaging molecule are Fab fragments. In another embodiment, one binding domain of the PSMA-targeted T-cell engaging molecule is a Fab fragment and the other binding domain is a scFv. In yet another embodiment, both binding domains of the PSMA-targeted T-cell engaging molecule are scFvs. In still another embodiment, one binding domain of the PSMA-targeted T-cell engaging molecule is a nanobody and the other binding domain is a Fab fragment.

As used in the context of the invention, an “antigen-binding fragment,” used interchangeably herein with “binding fragment” or “fragment,” is a portion of an antibody that lacks at least some of the amino acids present in a full-length heavy chain and/or light chain, but which is still capable of specifically binding to an antigen. An antigen-binding fragment includes, but is not limited to, a single-chain variable fragment (scFv), a nanobody (e.g. VH domain of heavy chain only antibodies (e.g. camelid heavy chain antibodies); VHH fragment, see Cortez-Retamozo et al, Cancer Research, Vol. 64:2853-57, 2004), a Fab fragment, a Fab′ fragment, a F(ab′)₂ fragment, a Fv fragment, a Fd fragment, and a CDR fragment, and can be derived from any mammalian source, such as human, mouse, rat, rabbit, or camelid. Antigen-binding fragments may compete for binding of a target antigen with an intact antibody and the fragments may be produced by the modification of intact antibodies (e.g. enzymatic or chemical cleavage) or synthesized de novo using recombinant DNA technologies or peptide synthesis. In some embodiments, the antigen-binding fragment comprises at least one CDR from an antibody that binds to the antigen, for example, the heavy chain CDR3 from an antibody that binds to the antigen. In other embodiments, the antigen-binding fragment comprises all three CDRs from the heavy chain of an antibody that binds to the antigen or all three CDRs from the light chain of an antibody that binds to the antigen. In still other embodiments, the antigen-binding fragment comprises all six CDRs from an antibody that binds to the antigen (three from the heavy chain and three from the light chain).

Papain digestion of antibodies produces two identical antigen-binding fragments, called “Fab” fragments, each with a single antigen-binding site, and a residual “Fc” fragment which contains all but the first domain of the immunoglobulin heavy chain constant region. The Fab fragment contains the variable domains from the light and heavy chains, as well as the constant domain of the light chain and the first constant domain (CH1) of the heavy chain. Thus, a “Fab fragment” is comprised of one immunoglobulin light chain (light chain variable region (VL) and constant region (CL)) and the CHI domain and variable region (VH) of one immunoglobulin heavy chain. The heavy chain of a Fab molecule cannot form a disulfide bond with another heavy chain molecule. The “Fd fragment” comprises the VH and CHI domains from an immunoglobulin heavy chain. The Fd fragment represents the heavy chain component of the Fab fragment.

The “Fc fragment” or “Fc domain” of an immunoglobulin generally comprises two constant domains, a CH2 domain and a CH3 domain, and optionally comprises a CH4 domain. In certain embodiments, the PSMA-targeted T-cell engaging molecules used in the methods of the invention comprise an Fc domain from an immunoglobulin. The Fc domain may be an Fc domain from an IgG1, IgG2, IgG3, or IgG4 immunoglobulin. In some embodiments, the Fc domain comprises CH2 and CH3 domains from a human IgG1 or human IgG2 immunoglobulin. The Fc domain may retain effector function, such as C1q binding, complement dependent cytotoxicity (CDC), Fc receptor binding, antibody-dependent cell-mediated cytotoxicity (ADCC), and phagocytosis. In other embodiments, the Fc domain may be modified to reduce or eliminate effector function.

A “Fab′ fragment” is a Fab fragment having at the C-terminus of the CH1 domain one or more cysteine residues from the antibody hinge region.

A “F(ab′)₂ fragment” is a bivalent fragment including two Fab′ fragments linked by a disulfide bridge between the heavy chains at the hinge region.

The “Fv” fragment is the minimum fragment that contains a complete antigen recognition and binding site from an antibody. This fragment consists of a dimer of one immunoglobulin heavy chain variable region (VH) and one immunoglobulin light chain variable region (VL) in tight, non-covalent association. It is in this configuration that the three CDRs of each variable region interact to define an antigen binding site on the surface of the VH-VL dimer. A single light chain or heavy chain variable region (or half of an Fv fragment comprising only three CDRs specific for an antigen) has the ability to recognize and bind antigen, although at a lower affinity than the entire binding site comprising both VH and VL.

A “single-chain variable fragment” or “scFv fragment” comprises the VH and VL regions of an antibody, wherein these regions are present in a single polypeptide chain, and optionally comprising a peptide linker between the VH and VL regions that enables the Fv to form the desired structure for antigen binding (see e.g., Bird et al., Science, Vol. 242:423-426, 1988; and Huston et al., Proc. Natl. Acad. Sci. USA, Vol. 85:5879-5883, 1988).

A “nanobody” is the heavy chain variable region of a heavy-chain antibody. Such variable domains are the smallest fully functional antigen-binding fragment of such heavy-chain antibodies with a molecular mass of only 15 kDa. See Cortez-Retamozo et al., Cancer Research 64:2853-57, 2004. Functional heavy-chain antibodies devoid of light chains are naturally occurring in certain species of animals, such as nurse sharks, wobbegong sharks and Camelidae, such as camels, dromedaries, alpacas and llamas. The antigen-binding site is reduced to a single domain, the VHH domain, in these animals. These antibodies form antigen-binding regions using only heavy chain variable region, i.e., these functional antibodies are homodimers of heavy chains only (referred to as “heavy-chain antibodies” or “HCAbs”). Camelized VHH reportedly recombines with IgG2 and IgG3 constant regions that contain hinge, CH2, and CH3 domains and lack a CH1 domain. Camelized VHH domains have been found to bind to antigen with high affinity (Desmyter et al., J. Biol. Chem., Vol. 276.26285-90, 2001) and possess high stability in solution (Ewert et al., Biochemistry, Vol. 41:3628-36, 2002). Methods for generating antibodies having camelized heavy chains are described in, for example, U.S. Patent Publication Nos. 2005/0136049 and 2005/0037421. Alternative scaffolds can be made from human variable-like domains that more closely match the shark V-NAR scaffold and may provide a framework for a long penetrating loop structure. Human heavy-chain antibodies can be produced by transgenic animals expressing human immunoglobulin genes, such as Uni Ab™ antibodies produced by UniRat™ transgenic rats.

In certain embodiments, the binding domains of the PSMA-targeted T-cell engaging molecules used in the methods of the invention comprise an immunoglobulin heavy chain variable region (VH) and an immunoglobulin light chain variable region (VL) of an antibody or antibody fragment which specifically binds to the desired antigen. For instance, the anti-PSMA binding domain of the PSMA-targeted T-cell engaging molecules of the invention comprises a VH region and VL region from an anti-PSMA antibody, such as any of the anti-PSMA antibodies or fragments thereof known in the art or described herein, and the anti-CD3 binding domain comprises a VH region and VL region from an anti-CD3 antibody, such as any of the anti-CD3 antibodies or fragments thereof known in the art or described herein. The binding domains that specifically bind to human PSMA or human CD3 can be derived from known antibodies to these antigens or from new antibodies or antibody fragments obtained by de novo immunization methods using the antigen proteins or fragments thereof, by phage display, or other methods described herein or known in the art. The antibodies from which the binding domains for the PSMA-targeted T-cell engaging molecules are derived can be monoclonal antibodies, recombinant antibodies, chimeric antibodies, human antibodies, or humanized antibodies. In certain embodiments, the antibodies from which the binding domains are derived are monoclonal antibodies. In these and other embodiments, the antibodies are human antibodies or humanized antibodies and can be of the IgG1-, IgG2-, IgG3-, or IgG4-type.

The first binding domain of the PSMA-targeted T-cell engaging molecules used in the methods of the invention specifically binds to PSMA, preferably human PSMA. This binding domain is referred to herein as an anti-PSMA binding domain. PSMA (prostate-specific membrane antigen; also known as glutamate carboxypeptidase II (GCPII), N-acetyl-L-aspartyl-L-glutamate peptidase I, or NAAG peptidase) is a type II membrane glycoprotein expressed primarily on prostate epithelial cells. More preferably, the first binding domain binds to PSMA on the surface of a target cell. The “target cell” can be any prokaryotic or eukaryotic cell expressing PSMA on its surface; preferably the target cell is a cell that is part of the human or animal body, such as a specific PSMA-expressing cancer or tumor cell. It is furthermore envisaged that the first binding domain of the PSMA-targeted T-cell engaging molecules binds to human PSMA, preferably to human PSMA on the surface of a target cell. It is also envisaged that the first binding domain binds to macaque PSMA, preferably to macaque PSMA on the surface of a target cell. Exemplary amino acid sequences for the mature polypeptides and extracellular domains of human PSMA and macaque PSMA are provided in Table I below

TABLE 1
Sequences of human and macaque PSMA polypeptides

| Polypeptide | Sequence |
|---|---|
| Human PSMA | MWNLLHETDSAVATARRPRWLCAGALVLAGGFFLLGFLFGWFIKSSN |
| | EATNITPKHNMKAFLDELKAENIKKFLYNFTQIPHLAGTEQNFQLAK |
| | QIQSQWKEFGLDSVELAHYDVLLSYPNKTHPNYISIINEDGNEIFNT |
| | SLFEPPPPGYENVSDIVPPFSAFSPQGMPEGDLVYVNYARTEDFFKL |
| | ERDMKINCSGKIVIARYGKVFRGNKVKNAQLAGAKGVILYSDPADYF |
| | APGVKSYPDGWNLPGGGVQRGNILNLNGAGDPLTPGYPANEYAYRRG |
| | IAEAVGLPSIPVHPIGYYDAQKLLEKMGGSAPPDSSWRGSLKVPYNV |
| | GPGFTGNFSTQKVKMHIHSTNEVTRIYNVIGTLRGAVEPDRYVILGG |
| | HRDSWVFGGIDPQSGAAVVHEIVRSFGTLKKEGWRPRRTILFASWDA |
| | EEFGLLGSTEWAEENSRLLQERGVAYINADSSIEGNYTLRVDCTPLM |
| | YSLVHNLTKELKSPDEGFEGKSLYESWTKKSPSPEFSGMPRISKLGS |
| | GNDFEVFFQRLGIASGRARYTKNWETNKFSGYPLYHSVYETYELVEK |
| | FYDPMFKYHLTVAQVRGGMVFELANSIVLPFDCRDYAVVLRKYADKI |
| | YSISMKHPQEMKTYSVSFDSLFSAVKNFTEIASKFSERLQDFDKSNP |
| | IVLRMMNDQLMFLERAFIDPLGLPDRPFYRHVIYAPSSHNKYAGESF |
| | PGIYDALFDIESKVDPSKAWGEVKRQIYVAAFTVQAAAETLSEVA |
| | (SEQ ID NO: 1) |
| Macaque PSMA | MWNLLHETDSAVATARRPRWLCAGALVLAGGFFLLGFLFGWFIKSSS |
| | EATNITPKHNMKAFLDELKAENIKKFLHNFTQIPHLAGTEQNFQLAK |
| | QIQSQWKEFGLDSVELTHYDVLLSYPNKTHPNYISIINEDGNEIFNT |
| | SLFEPPPAGYENVSDIVPPFSAFSPOGMPEGDLVYVNYARTEDFFKL |
| | ERDMKINCSGKIVIARYGKVFRGNKVKNAQLAGATGVILYSDPADYF |
| | APGVKSYPDGWNLPGGGVQRGNILNLNGAGDPLTPGYPANEYAYRRG |
| | MAEAVGLPSIPVHPIGYYDAQKLLEKMGGSASPDSSWRGSLKVPYNV |
| | GPGFTGNFSTQKVKMHIHSTSEVTRIYNVIGTLRGAVEPDRYVILGG |
| | HRDSWVFGGIDPQSGAAVVHEIVRSFGTLKKEGWRPRRTILFASWDA |
| | EEFGLLGSTEWAEENSRLLQERGVAYINADSSIEGNYTLRVDCTPLM |
| | YSLVYNLTKELESPDEGFEGKSLYESWTKKSPSPEFSGMPRISKLGS |
| | GNDFEVFFQRLGIASGRARYTKNWETNKFSSYPLYHSVYETYELVEK |
| | FYDPMFKYHLTVAQVRGGMVFELANSVVLPFDCRDYAVVLRKYADKI |
| | YNISMKHPQEMKTYSVSFDSLFSAVKNFTEIASKFSERLRDFDKSNP |
| | ILLRMMNDQLMFLERAFIDPLGLPDRPFYRHVIYAPSSHNKYAGESF |
| | PGIYDALFDIESKVDPSQAWGEVKRQISVATFTVQAAAETLSEVA |
| | (SEQ ID NO: 2) |
| Extracellular | KSSNEATNITPKHNMKAFLDELKAENIKKFLYNFTQIPHLAGTEQNF |
| Domain of | QLAKQIQSQWKEFGLDSVELAHYDVLLSYPNKTHPNYISIINEDGNE |
| Human PSMA | IFNTSLFEPPPPGYENVSDIVPPFSAFSPQGMPEGDLVYVNYARTED |
| | FFKLERDMKINCSGKIVIARYGKVFRGNKVKNAQLAGAKGVILYSDP |
| | ADYFAPGVKSYPDGWNLPGGGVQRGNILNLNGAGDPLTPGYPANEYA |
| | YRRGIAEAVGLPSIPVHPIGYYDAQKLLEKMGGSAPPDSSWRGSLKV |
| | PYNVGPGFTGNFSTQKVKMHIHSTNEVTRIYNVIGTLRGAVEPDRYV |
| | ILGGHRDSWVFGGIDPQSGAAVVHEIVRSFGTLKKEGWRPRRTILFA |
| | SWDAEEFGLLGSTEWAEENSRLLQERGVAYINADSSIEGNYTLRVDC |
| | TPLMYSLVHNLTKELKSPDEGFEGKSLYESWTKKSPSPEFSGMPRIS |
| | KLGSGNDFEVFFQRLGIASGRARYTKNWETNKFSGYPLYHSVYETYE |
| | LVEKFYDPMFKYHLTVAQVRGGMVFELANSIVLPFDCRDYAVVLRKY |
| | ADKIYSISMKHPQEMKTYSVSFDSLFSAVKNFTEIASKFSERLQDFD |
| | KSNPIVLRMMNDQLMFLERAFIDPLGLPDRPFYRHVIYAPSSHNKYA |
| | GESFPGIYDALFDIESKVDPSKAWGEVKRQIYVAAFTVQAAAETLSE |
| | VA (SEQ ID NO: 3) |
| Extracellular | KSSSEATNITPKHNMKAFLDELKAENIKKFLHNFTQIPHLAGTEQNF |
| Domain of | QLAKQIQSQWKEFGLDSVELTHYDVLLSYPNKTHPNYISIINEDGNE |
| Macaque PSMA | IFNTSLFEPPPAGYENVSDIVPPFSAFSPQGMPEGDLVYVNYARTED |
| | FFKLERDMKINCSGKIVIARYGKVFRGNKVKNAQLAGATGVILYSDP |
| | ADYFAPGVKSYPDGWNLPGGGVQRGNILNLNGAGDPLTPGYPANEYA |
| | YRRGMAEAVGLPSIPVHPIGYYDAQKLLEKMGGSASPDSSWRGSLKV |
| | PYNVGPGFTGNFSTQKVKMHIHSTSEVTRIYNVIGTLRGAVEPDRYV |
| | ILGGHRDSWVFGGIDPQSGAAVVHEIVRSFGTLKKEGWRPRRTILFA |
| | SWDAEEFGLLGSTEWAEENSRLLQERGVAYINADSSIEGNYTLRVDC |
| | TPLMYSLVYNLTKELESPDEGFEGKSLYESWTKKSPSPEFSGMPRIS |
| | KLGSGNDFEVFFQRLGIASGRARYTKNWETNKFSSYPLYHSVYETYE |
| | LVEKFYDPMFKYHLTVAQVRGGMVFELANSVVLPFDCRDYAVVLRKY |
| | ADKIYNISMKHPQEMKTYSVSFDSLFSAVKNFTEIASKFSERLRDFD |
| | KSNPILLRMMNDQLMFLERAFIDPLGLPDRPFYRHVIYAPSSHNKYA |
| | GESFPGIYDALFDIESKVDPSQAWGEVKRQISVATFTVQAAAETLSE |
| | VA (SEQ ID NO: 4) |

Examples of anti-PSMA binding domains from which the first binding domain of the PSMA-targeted T-cell engaging molecules used in the methods of the invention can be constructed or derived are described in WO 2010/037836, WO 2011/121110, WO 2017/134158, and WO2020/206330, all of which are hereby incorporated by reference in their entireties. Light chain and heavy chain variable regions and associated CDRs of exemplary anti-human PSMA antibodies from which the anti-PSMA binding domain of the PSMA-targeted T-cell engaging molecules can be derived or constructed are set forth in Tables 2A and 2B, respectively.

TABLE 2A
Exemplary Anti-Human PSMA Antibody Light Chain Variable
Region Amino Acid Sequences

| Antibody | VL | VL Amino Acid | | | |
|---|---|---|---|---|---|
| ID. | Group | Sequence | CDRL1 | CDRL2 | CDRL3 |
| 01 | LV-01 | DIQMTQSPSSLSASVGDRV | KASQNVDTNVA | SASYRYS | QQYDQQLIT |
| | | ITCKASQNVDTNVAWYQQT | (SEQ ID | (SEQ ID | (SEQ ID |
| | | KPGQAPKSLIYSASYRYSD | NO: 5) | NO: 7) | NO: 9) |
| | | VPSRFSGSASGTDFTLTIS | | | |
| | | SVQSEDFATYYCQQYDQQL | | | |
| | | ITFGCGTKLEIK | | | |
| | | (SEQ ID NO: 21) | | | |
| 02 | LV-02 | DIQMTQSPSSLSASVGDRV | KASQNVDTNVA | SASYRYS | QQYDQVRLT |
| | | TITCKASQNVDTNVAWYQQ | (SEQ ID | (SEQ ID | (SEQ ID |
| | | KPGQAPKSLIYSASYRYSD | NO: 5) | NO: 7) | NO: 10) |
| | | VPSRFSGSASGTDFTLTIS | | | |
| | | SVQSEDFATYYCQQYDQVR | | | |
| | | LTFGCGTKLEIK | | | |
| | | (SEQ ID NO: 22) | | | |
| 03, 09 | LV-03 | DIQMTQSPSSLSASVGDRV | KASQNVDTNVA | SASYRYS | QQYDSYPYT |
| | | TITCKASQNVDTNVAWYQQ | (SEQ ID | (SEQ ID | (SEQ ID |
| | | KPGQAPKSLIYSASYRYSD | NO: 5) | NO: 7) | NO: 11) |
| | | VPSRFSGSASGTDFTLTIS | | | |
| | | SVQSEDFATYYCQQYDSYP | | | |
| | | YTFGCGTKLEIK | | | |
| | | (SEQ ID NO: 23) | | | |
| 04 | LV-04 | DIQMTQSPSSLSASVGDRV | KASQNVDTNVA | SASYRYS | QQYDROPLT |
| | | TITCKASQNVDINVAWYQQ | (SEQ ID | (SEQ ID | (SEQ ID |
| | | KPGQAPKSLIYSASYRYSD | NO: 5) | NO: 7) | NO: 12) |
| | | VPSRFSGSASGTDFTLTIS | | | |
| | | SVQSEDFATYYCQQYDRQP | | | |
| | | LTFGCGTKLEIK | | | |
| | | (SEQ ID NO: 24) | | | |
| 05 | LV-05 | DIQMTQSPSSLSASVGDRV | KASQNVDTNVA | SASYRYS | QQYDAPMGT |
| | | TITCKASQNVDTNVAWYQQ | (SEQ ID | (SEQ ID | (SEQ ID |
| | | KPGQAPKSLIYSASYRYSD | NO: 5) | NO: 7) | NO: 13) |
| | | VPSRFSGSASGTDFTLTIS | | | |
| | | SVQSEDFATYYCQQYDAPM | | | |
| | | GTFGCGTKLEIK | | | |
| | | (SEQ ID NO: 25) | | | |
| 06 | LV-06 | DIQMTOSPSSLSASVGDRV | KASQNVDTNVA | SASYRYS | QQYDSYPYT |
| | | TITCKASQNVDTNVAWYQQ | (SEQ ID | (SEQ ID | (SEQ ID |
| | | KPGQAPKSLIYSASYRYSD | NO: 5) | NO: 7) | NO: 11) |
| | | VPSRFSGSASGTDFTLTIS | | | |
| | | SLQSEDFATYYCQQYDSYP | | | |
| | | YTFGGGTKLEIK | | | |
| | | (SEQ ID NO: 26) | | | |
| 07 | LV-07 | DIQMTQSPSSLSASVGDRV | KASQNVDTNVA | SASYRYS | QQYDSYPYT |
| | | TITCKASQNVDTNVAWYQQ | (SEQ ID | (SEQ ID | (SEQ ID |
| | | KPGQAPKSLIYSASYRYSD | NO: 5) | NO: 7) | NO: 11) |
| | | VPSRFSGSASGTDFTLTIS | | | |
| | | SLQSEDFATYYCQQYDSYP | | | |
| | | YTFGCGTKLEIK | | | |
| | | (SEQ ID NO: 27) | | | |
| 08 | LV-08 | DIQMTOSPSSLSASVGDRV | KASQNVDTNVA | SASYRYS | QQYDSYPYT |
| | | TITCKASQNVDTNVAWYQQ | (SEQ ID | (SEQ ID | (SEQ ID |
| | | KPGQAPKSLIYSASYRYSD | NO: 5) | NO: 7) | NO: 11) |
| | | VPSRFSGSASGTDFTLTIS | | | |
| | | SVQSEDFATYYCQQYDSYP | | | |
| | | YTFGGGTKLEIK | | | |
| | | (SEQ ID NO: 28) | | | |
| 10 | LV-09 | DIQMTQSPSSLSASVGDRV | KASQNVDTNVA | SASYVYW | QQYDQQLIT |
| | | TITCKASQNVDTNVAWYQQ | (SEQ ID | (SEQ ID | (SEQ ID |
| | | KPGQAPKSLIYSASYVYWD | NO: 5) | NO: 8) | NO: 9) |
| | | VPSRFSGSASGTDFTLTIS | | | |
| | | SVQSEDFATYYCQQYDQQL | | | |
| | | ITFGGGTKLEIK (SEQ | | | |
| | | ID NO: 29) | | | |
| 11 | LV-10 | DIQMTQSPSSLSASVGDRV | KASQNVDTNVA | SASYVYW | QQYDQQLIT |
| | | TITCKASQNVDTNVAWYQQ | (SEQ ID | (SEQ ID | (SEQ ID |
| | | KPGQAPKSLIYSASYVYWD | NO: 5) | NO: 8) | NO: 9) |
| | | VPSRFSGSASGTDFTLTIS | | | |
| | | SVQSEDFATYYCQQYDQQL | | | |
| | | ITFGCGTKLEIK (SEQ | | | |
| | | ID NO: 30) | | | |
| 12 | LV-11 | DIQMTQSPSSLSASVGDRV | KASQNVDANVA | SASYVYW | QQYDQQLIT |
| | | TITCKASQNVDANVAWYQQ | (SEQ ID | (SEQ ID | (SEQ ID |
| | | KPGQAPKSLIYSASYVYWD | NO: 6) | NO: 8) | NO: 9) |
| | | VPSRFSGSASGTDFTLTIS | | | |
| | | SVQSEDFATYYCQQYDQQL | | | |
| | | ITFGGGTKLEIK (SEQ | | | |
| | | ID NO: 31) | | | |
| 13 | LV-12 | DIQMTQSPSSLSASVGDRV | KASQNVDANVA | SASYVYW | QQYDQQLIT |
| | | TITCKASQNVDANVAWYQQ | (SEQ ID | (SEQ ID | (SEQ ID |
| | | KPGQAPKSLIYSASYVYWD | NO: 6) | NO: 8) | NO: 9) |
| | | VPSRFSGSASGTDFTLTIS | | | |
| | | SVQSEDFATYYCQQYDQQL | | | |
| | | ITFGCGTKLEIK (SEQ | | | |
| | | ID NO: 32) | | | |

TABLE 2B
Exemplary Anti-Human PSMA Antibody Heavy Chain Variable Region Amino
Acid Sequences

| Antibody | VH | VH Amino Acid | | | |
|---|---|---|---|---|---|
| ID. | Group | Sequence | CDRH1 | CDRH2 | CDRH3 |
| 01, 02, 04, | HV-01 | QVQLVESGGGLVKPGE | DYYMY | IISDGGYYTYYSDIIKG | GFPLLRHGAM |
| 05, 09, 11 | | SLRLSCAASGFTFSDYY | (SEQ ID NO: | (SEQ ID NO: 16) | DY |
| | | MYWVRQAPGKCLEWV | 14) | | (SEQ ID NO: |
| | | AUSDGGYYTYYSDIIKG | | | 20) |
| | | RFTISRDNAKNSLYLQ | | | |
| | | MNSLKAEDTAVYYCA | | | |
| | | RGFPLLRHGAMDYWG | | | |
| | | QGTLVTVSS (SEQ ID | | | |
| | | NO: 33) | | | |
| 03 | HV-02 | QVQLVESGGGLVKPGE | DYYMY | IISDHHHWTYYSDIIKG | GFPLLRHGAM |
| | | SLRLSCAASGFTFSDYY | (SEQ ID NO: | (SEQ ID NO: 17) | DY |
| | | MYWVRQAPGKCLEWV | 14) | | (SEQ ID NO: |
| | | AIISDHHHWTYYSDIIK | | | 20) |
| | | GRFTISRDNAKNSLYLQ | | | |
| | | MNSLKAEDTAVYYCA | | | |
| | | RGFPLLRHGAMDYWG | | | |
| | | QGTLVTVSS (SEQ ID | | | |
| | | NO: 34) | | | |
| 06 | HV-03 | QVQLVESGGGLVKPGE | DYYMN | ISDNKHQTYYSDIIKG | GFPLLRHGAM |
| | | SLRLSCAASGFTFSDYY | (SEQ ID NO: | (SEQ ID NO: 18) | DY |
| | | MNWVRQAPGKGLEWV | 15) | | (SEQ ID NO: |
| | | AIISDNKHQTYYSDIIKG | | | 20) |
| | | RFAISRDNAKNSLYLQ | | | |
| | | MNSLKAEDTAVYYCA | | | |
| | | RGFPLLRHGAMDYWG | | | |
| | | QGTLVTVSS (SEQ ID | | | |
| | | NO: 35) | | | |
| 07 | HV-04 | QVQLVESGGGLVKPGE | DYYMN | IISDNKHQTYYSDIIKG | GFPLLRHGAM |
| | | SLRLSCAASGFTFSDYY | (SEQ ID NO: | (SEQ ID NO: 18) | DY |
| | | MNWVRQAPGKCLEWV | 15) | | (SEQ ID NO: |
| | | AIISDNKHQTYYSDIIKG | | | 20) |
| | | RFAISRDNAKNSLYLQ | | | |
| | | MNSLKAEDTAVYYCA | | | |
| | | RGFPLLRHGAMDYWG | | | |
| | | QGTLVTVSS (SEQ ID | | | |
| | | NO: 36) | | | |
| 08, 10 | HV-05 | QVQLVESGGGLVKPGE | DYYMY | IISDGGYYTYYSDIIKG | GFPLLRHGAM |
| | | SLRLSCAASGFTFSDYY | (SEQ ID NO: | (SEQ ID NO: 16) | DY |
| | | MYWVRQAPGKGLEWV | 14) | | (SEQ ID NO: |
| | | AIISDGGYYTYYSDIIKG | | | 20) |
| | | RFTISRDNAKNSLYLQ | | | |
| | | MNSLKAEDTAVYYCA | | | |
| | | RGFPLLRHGAMDYWG | | | |
| | | QGTLVTVSS (SEQ ID | | | |
| | | NO: 37) | | | |
| 12 | HV-06 | QVQLVESGGGLVKPGE | DYYMY | IISDAGYYTYYSDIIKG | GFPLLRHGAM |
| | | SLRLSCAASGFTFSDYY | (SEQ ID NO: | (SEQ ID NO: 19) | DY |
| | | MYWVRQAPGKGLEWV | 14) | | (SEQ ID NO: |
| | | AIISDAGYYTYYSDIIKG | | | 20) |
| | | RFTISRDNAKNSLYLQ | | | |
| | | MNSLKAEDTAVYYCA | | | |
| | | RGFPLLRHGAMDYWG | | | |
| | | QGTLVTVSS (SEQ ID | | | |
| | | NO: 38) | | | |
| 13 | HV-07 | QVQLVESGGGLVKPGE | DYYMY | IISDAGYYTYYSDIIKG | GFPLLRHGAM |
| | | SLRLSCAASGFTFSDYY | (SEQ ID NO: | (SEQ ID NO: 19) | DY |
| | | MYWVRQAPGKCLEWV | 14) | | (SEQ ID NO: |
| | | AIISDAGYYTYYSDIIKG | | | 20) |
| | | RFTISRDNAKNSLYLQ | | | |
| | | MNSLKAEDTAVYYCA | | | |
| | | RGFPLLRHGAMDYWG | | | |
| | | QGTLVTVSS (SEQ ID | | | |
| | | NO: 39) | | | |
| 51 | HV-08 | QLQLQESGPGLVKPSET | GGSISSSNYF | IDYSGYT | ARHKAATADF |
| | | LSLTCTVSGGSISSSNYF | (SEQ ID NO: | (SEQ ID NO: 163) | DY |
| | | WGWIRQSPGKGLEWIG | 158) | | (SEQ ID NO: |
| | | SIDYSGYTYYNPSLKSR | | | 167 |
| | | VTISVDTSKNQFSLKLS | | | |
| | | SVTAADTAVYNCARHK | | | |
| | | AATADFDYRGQGTLVT | | | |
| | | VSS (SEQ ID NO: 170) | | | |
| 52 | HV-09 | QVQLVESGGGVVQPGR | GFSFSRYG | IWYDGSNR | AREPRIGYYY |
| | | SLRLSCAASGFSFSRYG | (SEQ ID NO: | (SEQ ID NO: 164) | ESSGYYSLDY |
| | | MHWVRQAPGKGLEGV | 159) | | (SEQ ID NO: |
| | | AVIWYDGSNRYYADSV | | | 168) |
| | | KGRFTISRDNSKNTLYL | | | |
| | | QMNSLRAEDTAVYYC | | | |
| | | AREPRIGYYYESSGYYS | | | |
| | | LDYRGQGTLVTVSS | | | |
| | | (SEQ ID NO: 171) | | | |
| 53 | HV-10 | QLQLQESGPGLVKPSET | GGSISSSSYY | IDYSGYT | ARHKAATADF |
| | | LSLTCTVSGGSISSSSYY | (SEQ ID NO: | (SEQ ID NO: 163) | DY |
| | | WGWIRQPPGKGLEWIG | 160) | | (SEQ ID NO: |
| | | SIDYSGYTYYNPSLQSR | | | 167) |
| | | VTISVDTSKNQFSLKLS | | | |
| | | SVTAADTAVYNCARHK | | | |
| | | AATADFDYRGQGTLVT | | | |
| | | VSS (SEQ ID NO: 172) | | | |
| 54 | HV-11 | QLQLQESGPGLVKPSET | GGSISSSSYY | VDYSGYT | ARHKAATADF |
| | | LSLTCTVSGGSISSSSYY | (SEQ ID NO: | (SEQ ID NO: 165) | DY |
| | | WGWIRQPPGKGLEWIG | 160) | | (SEQ ID NO: |
| | | SVDYSGYTYYNPSLQS | | | 167) |
| | | RVTISVDTSKNQFSLKL | | | |
| | | SSVTAADTAVYNCARH | | | |
| | | KAATADFDYRGQGTLV | | | |
| | | TVSS (SEQ ID NO: 173) | | | |
| 55 | HV-12 | QLQLQESGPGLVKPSET | GGSISSSSYY | IDYSGYT | ARHKAATADF |
| | | LSLTCTVSGGSISSSSYY | (SEQ ID NO: | (SEQ ID NO: 163) | DY |
| | | WGWIRQPPGKGLEWIG | 160) | | (SEQ ID NO: |
| | | SIDYSGYTYYNPSLKSR | | | 167) |
| | | VTISVDTSKNQFSLKLS | | | |
| | | SVTAADTAVYNCARHK | | | |
| | | AATADFDYRGQGTLVT | | | |
| | | VSS (SEQ ID NO: 174) | | | |
| 56 | HV-13 | QVQLVESGGGVVQPGR | GFSFRSYG | IWYDGSNK | AREPRIGYYY |
| | | SLRLSCAASGFSFRSYG | (SEQ ID NO: | (SEQ ID NO: 166) | ESSGYDSLDY |
| | | MHWVRQAPGKGLEWV | 161) | | (SEQ ID NO: |
| | | AVIWYDGSNKYYADSV | | | 169) |
| | | KGRFTISRDYSKNTLYL | | | |
| | | QMNSLRAEDTAVYYC | | | |
| | | AREPRIGYYYESSGYDS | | | |
| | | LDYRGQGTLVTVSS | | | |
| | | (SEQ ID NO: 175) | | | |
| 57 | HV-14 | QVQLVESGGGVVQPGR | GFSFSSYG | IWYDGSNK | AREPRIGYYY |
| | | SLRLSCAASGFSFSSYG | (SEQ ID NO: | (SEQ ID NO: 166) | ESSGYDSLDY |
| | | MHWVRQAPGKGLEWV | 162) | | (SEQ ID NO: |
| | | AVIWYDGSNKYYADSV | | | 169) |
| | | KGRFTISRDYSKNTLYL | | | |
| | | QMNSLRAEDTAVYYC | | | |
| | | AREPRIGYYYESSGYDS | | | |
| | | LDYRGOGTLVTVSS | | | |
| | | (SEQ ID NO: 176) | | | |

The domain that specifically binds to human PSMA (e.g., the anti-PSMA binding domain) of the PSMA-targeted T-cell engaging molecules suitable for use in the methods of the invention may comprise one or more of the light chain CDRs (i.e. CDRLs) and/or heavy chain CDRs (i.e. CDRHs) presented in Tables 2A and 2B, respectively. For instance, in some embodiments, the anti-PSMA binding domains of the PSMA-targeted T-cell engaging molecules according to the invention comprise a CDRL1 comprising the sequence of SEQ ID NO: 5 or SEQ ID NO: 6; a CDRL2 comprising the sequence of SEQ ID NO: 7 or SEQ ID NO. 8; a CDRL3 comprising a sequence selected from SEQ ID NOs: 9 to 13; a CDRH1 comprising the sequence of SEQ ID NO: 14 or SEQ ID NO: 15; a CDRH2 comprising a sequence selected from SEQ ID NOs: 16 to 19; and a CDRH3 comprising the sequence of SEQ ID NO: 20.

In some embodiments, the anti-PSMA binding domains of the PSMA-targeted T-cell engaging molecules comprise a light chain variable region comprising a CDRL1, a CDRL2, and a CDRL3, wherein: (a) CDRL1, CDRL2, and CDRL3 have the sequence of SEQ ID NOs: 5, 7 and 9, respectively; (b) CDRL1, CDRL2, and CDRL3 have the sequence of SEQ ID NOs: 5, 7 and 10, respectively; (c) CDRL1, CDRL2, and CDRL3 have the sequence of SEQ ID NOs: 5, 7 and 11, respectively; (d) CDRL1, CDRL2, and CDRL3 have the sequence of SEQ ID NOs: 5, 7 and 12, respectively; (e) CDRL1, CDRL2, and CDRL3 have the sequence of SEQ ID NOs: 5, 7 and 13, respectively, (f) CDRL1, CDRL2, and CDRL3 have the sequence of SEQ ID NOs: 5, 8 and 9, respectively; or (g) CDRL1, CDRL2, and CDRL3 have the sequence of SEQ ID NOs: 6, 8 and 9, respectively. In these and other embodiments, the anti-PSMA binding domains of the PSMA-targeted T-cell engaging molecules comprise a heavy chain variable region comprising a CDRH1, a CDRH2, and a CDRH3, wherein: (a) CDRH1, CDRH2, and CDRH3 have the sequence of SEQ ID NOs: 14, 16 and 20, respectively; (b) CDRH1, CDRH2, and CDRH3 have the sequence of SEQ ID NOs: 14, 17 and 20, respectively; (c) CDRH1, CDRH2, and CDRH3 have the sequence of SEQ ID NOs: 15, 18 and 20, respectively; or (d) CDRH1, CDRH2, and CDRH3 have the sequence of SEQ ID NOs: 14, 19 and 20, respectively.

In certain embodiments, the anti-PSMA binding domains of the PSMA-targeted T-cell engaging molecules suitable for use in the methods of the invention comprise a light chain variable region comprising a CDRL1, a CDRL2, and a CDRL3 and a heavy chain variable region comprising a CDRH1, a CDRH2, and a CDRH3, wherein:
(a) CDRL1, CDRL2, and CDRL3 have the sequence of SEQ ID NOs: 5, 7 and 9, respectively, and CDRH1, CDRH2, and CDRH3 have the sequence of SEQ ID NOs: 14, 16 and 20, respectively;
(b) CDRL1, CDRL2, and CDRL3 have the sequence of SEQ ID NOs: 5, 7 and 10, respectively, and CDRH1, CDRH2, and CDRH3 have the sequence of SEQ ID NOs: 14, 16 and 20, respectively;
(c) CDRL1, CDRL2, and CDRL3 have the sequence of SEQ ID NOs: 5, 7 and 11, respectively, and CDRH1, CDRH2, and CDRH3 have the sequence of SEQ ID NOs: 14, 17 and 20, respectively;
(d) CDRL1, CDRL2, and CDRL3 have the sequence of SEQ ID NOs: 5, 7 and 12, respectively, and CDRH1, CDRH2, and CDRH3 have the sequence of SEQ ID NOs: 14, 16 and 20, respectively;
(e) CDRL1, CDRL2, and CDRL3 have the sequence of SEQ ID NOs: 5, 7 and 13, respectively, and CDRH1, CDRH2, and CDRH3 have the sequence of SEQ ID NOs: 14, 16 and 20, respectively;
(f) CDRL1, CDRL2, and CDRL3 have the sequence of SEQ ID NOs: 5, 7 and 11, respectively, and CDRH1, CDRH2, and CDRH3 have the sequence of SEQ ID NOs: 15, 18 and 20, respectively;
(g) CDRL1, CDRL2, and CDRL3 have the sequence of SEQ ID NOs: 5, 7 and 11, respectively, and CDRH1, CDRH2, and CDRH3 have the sequence of SEQ ID NOs: 14, 16 and 20, respectively;
(h) CDRL1, CDRL2, and CDRL3 have the sequence of SEQ ID NOs: 5, 8 and 9, respectively, and CDRH1, CDRH2, and CDRH3 have the sequence of SEQ ID NOs: 14, 16 and 20, respectively; or
(i) CDRL1, CDRL2, and CDRL3 have the sequence of SEQ ID NOs: 6, 8 and 9, respectively, and CDRH1, CDRH2, and CDRH3 have the sequence of SEQ ID NOs: 14, 19 and 20, respectively. In a preferred embodiment, the anti-PSMA binding domain of the PSMA-targeted T-cell engaging molecules used in the methods of the invention comprises (i) a light chain variable region comprising a CDRL1 having the sequence of SEQ ID NO: 5, a CDRL2 having the sequence of SEQ ID NO: 8, and a CDRL3 having the sequence of SEQ ID NO: 9, and (ii) a heavy chain variable region comprising a CDRH1 having the sequence of SEQ ID NO: 14, a CDRH2 having the sequence of SEQ ID NO: 16, and a CDRH3 having the sequence of SEQ ID NO: 20.

In some embodiments, the anti-PSMA binding domain of the PSMA-targeted T-cell engaging molecules used in the methods of the invention comprise a heavy chain variable region from a heavy chain only antibody, such as the antibodies designated as antibodies 51 to 57 in Table 2B. In such embodiments, the anti-PSMA binding domain may comprise a heavy chain variable region comprising a CDRH1, a CDRH2, and a CDRH3, wherein: (a) CDRH1, CDRH2, and CDRH3 have the sequence of SEQ ID NOs: 158, 163, and 167, respectively; (b) CDRH1, CDRH2, and CDRH3 have the sequence of SEQ ID NOs: 159, 164, 168, respectively; (c) CDRH1, CDRH2, and CDRH3 have the sequence of SEQ ID NOs: 160, 163, and 167, respectively; (d) CDRH1, CDRH2, and CDRH3 have the sequence of SEQ ID NOs: 160, 165, and 167, respectively; (e) CDRH1, CDRH2, and CDRH3 have the sequence of SEQ ID NOs: 161, 166, and 169, respectively; or (f) CDRH1, CDRH2, and CDRH3 have the sequence of SEQ ID NOs: 162, 166, and 169, respectively.

In some embodiments, the anti-PSMA binding domain of the PSMA-targeted T-cell engaging molecules used in the methods of the invention comprise an immunoglobulin heavy chain variable region (VH) and an immunoglobulin light chain variable region (VL) from an antibody that specifically binds to human PSMA, such as the antibodies described herein. The “variable region,” used interchangeably herein with “variable domain” (variable region of a light chain (VL), variable region of a heavy chain (VH)), refers to the region in each of the light and heavy immunoglobulin chains which is involved directly in binding of the antibody to the antigen. As discussed above, the regions of variable light and heavy chains have the same general structure and each region comprises four framework (FR) regions, the sequences of which are widely conserved, connected by three CDRs. The framework regions adopt a beta-sheet conformation and the CDRs may form loops connecting the beta-sheet structure. The CDRs in each chain are held in their three-dimensional structure by the framework regions and form, together with the CDRs from the other chain, the antigen binding site. Thus, in some embodiments, the anti-PSMA binding domain of the PSMA-targeted T-cell engaging molecules for use according to the methods of the invention may comprise a light chain variable region selected from LV-01 to LV-12 (SEQ ID NOs: 21-32), as shown in Table 2A, and/or a heavy chain variable region selected from HV-01 to HV-14 (SEQ ID NOs: 33-39 and 170-176), as shown in Table 2B, and binding fragments, derivatives, and variants of these light chain and heavy chain variable regions. In certain embodiments, the anti-PSMA binding domain of the PSMA-targeted T-cell engaging molecules may comprise only a heavy chain variable region selected from HV-08 to HV-14 (SEQ ID NOs: 170-176), which correspond to heavy chain variable regions from heavy chain only antibodies 51 to 57 in Table 2B.

Each of the light chain variable regions listed in Table 2A may be combined with any of the heavy chain variable regions listed in Table 2B to form an anti-PSMA binding domain of the PSMA-targeted T-cell engaging molecules according to the invention. Examples of such combinations include, but are not limited to: (i) HV-01 and any one of LV-01, LV-02, LV-03, LV-04, LV-05 and LV-10; (ii) LV-03 and HV-02; (iii) LV-06 and HV-03; (iv) LV-07 and HV-04; (v) LV-08 and HV-05; (vi) LV-09 and HV-05; (vii) LV-11 and HV-06; and (viii) LV-12 and HV-07.

In certain embodiments, the anti-PSMA binding domains of the PSMA-targeted T-cell engaging molecules according to the invention comprise a light chain variable region comprising the sequence of SEQ ID NO: 21 and a heavy chain variable region comprising the sequence of SEQ ID NO: 33. In some embodiments, the anti-PSMA binding domains of the PSMA-targeted T-cell engaging molecules according to the invention comprise a light chain variable region comprising the sequence of SEQ ID NO: 22 and a heavy chain variable region comprising the sequence of SEQ ID NO: 33. In other embodiments, the anti-PSMA binding domains of the PSMA-targeted T-cell engaging molecules according to the invention comprise a light chain variable region comprising the sequence of SEQ ID NO: 23 and a heavy chain variable region comprising the sequence of SEQ ID NO: 33. In still other embodiments, the anti-PSMA binding domains of the PSMA-targeted T-cell engaging molecules according to the invention comprise a light chain variable region comprising the sequence of SEQ ID NO: 24 and a heavy chain variable region comprising the sequence of SEQ ID NO: 33. In some embodiments, the anti-PSMA binding domains of the PSMA-targeted T-cell engaging molecules according to the invention comprise a light chain variable region comprising the sequence of SEQ ID NO: 25 and a heavy chain variable region comprising the sequence of SEQ ID NO: 33. In certain embodiments, the anti-PSMA binding domains of the PSMA-targeted T-cell engaging molecules according to the invention comprise a light chain variable region comprising the sequence of SEQ ID NO: 23 and a heavy chain variable region comprising the sequence of SEQ ID NO: 34. In one embodiment, the anti-PSMA binding domains of the PSMA-targeted T-cell engaging molecules according to the invention comprise a light chain variable region comprising the sequence of SEQ ID NO: 26 and a heavy chain variable region comprising the sequence of SEQ ID NO: 35. In another embodiment, the anti-PSMA binding domains of the PSMA-targeted T-cell engaging molecules according to the invention comprise a light chain variable region comprising the sequence of SEQ ID NO: 27 and a heavy chain variable region comprising the sequence of SEQ ID NO: 36. In yet another embodiment, the anti-PSMA binding domains of the PSMA-targeted T-cell engaging molecules according to the invention comprise a light chain variable region comprising the sequence of SEQ ID NO: 31 and a heavy chain variable region comprising the sequence of SEQ ID NO: 38. In still another embodiment, the anti-PSMA binding domains of the PSMA-targeted T-cell engaging molecules according to the invention comprise a light chain variable region comprising the sequence of SEQ ID NO: 32 and a heavy chain variable region comprising the sequence of SEQ ID NO: 39. In some embodiments, the anti-PSMA binding domains of the PSMA-targeted T-cell engaging molecules according to the invention comprise a light chain variable region comprising the sequence of SEQ ID NO: 28 or SEQ ID NO: 29 and a heavy chain variable region comprising the sequence of SEQ ID NO: 37. In a preferred embodiment, the anti-PSMA binding domains of the PSMA-targeted T-cell engaging molecules according to the invention comprise a light chain variable region comprising the sequence of SEQ ID NO: 30 and a heavy chain variable region comprising the sequence of SEQ ID NO: 33.

In certain other embodiments, the anti-PSMA binding domains of the PSMA-targeted T-cell engaging molecules according to the invention comprise a heavy chain variable region comprising the sequence of any one of SEQ ID NOs: 170 to 176. In some such embodiments, the anti-PSMA binding domain does not comprise a light chain variable region. In one particular embodiment, the anti-PSMA binding domain comprises a heavy chain variable region comprising the sequence of SEQ ID NO: 170. In another particular embodiment, the anti-PSMA binding domain comprises a heavy chain variable region comprising the sequence of SEQ ID NO: 171. In another embodiment, the anti-PSMA binding domain comprises a first heavy chain variable region comprising the sequence of SEQ ID NO: 170 and a second heavy chain variable region comprising the sequence of SEQ ID NO: 171, wherein the two heavy chain variable region sequences are optionally connected by a linker (e.g. a glycine-serine linker).

In some embodiments, the anti-PSMA binding domains of the PSMA-targeted T-cell engaging molecules for use according to the methods of the invention comprise a light chain variable region comprising a sequence of contiguous amino acids that differs from the sequence of a light chain variable region in Table 2A, i.e. a VL selected from LV-01 to LV-12, at only 1, 2, 3, 4, 5, 6, 7, 8, 9, 10, 11, 12, 13, 14 or 15 amino acid residues, wherein each such sequence difference is independently either a deletion, insertion or substitution of one amino acid, with the deletions, insertions and/or substitutions resulting in no more than 15 amino acid changes relative to the foregoing variable domain sequences. The light chain variable region in some anti-PSMA binding domains comprises a sequence of amino acids that has at least 70%, at least 75%, at least 80%, at least 85%, at least 90%, at least 95%, at least 97% or at least 99% sequence identity to the amino acid sequences of SEQ ID NOs: 21 to 32 (i.e. the light chain variable regions in Table 2A).

In one embodiment, the anti-PSMA binding domains of the PSMA-targeted T-cell engaging molecules according to the invention comprise a light chain variable region comprising a sequence that is at least 90% identical to a sequence selected from SEQ ID NOs: 21-32. In another embodiment, the anti-PSMA binding domains of the PSMA-targeted T-cell engaging molecules according to the invention comprise a light chain variable region comprising a sequence that is at least 95% identical to a sequence selected from SEQ ID NOs: 21-32. In yet another embodiment, the anti-PSMA binding domains of the PSMA-targeted T-cell engaging molecules according to the invention comprise a light chain variable region comprising a sequence selected from SEQ ID NOs: 21-32.

In these and other embodiments, the anti-PSMA binding domains of the PSMA-targeted T-cell engaging molecules according to the invention comprise a heavy chain variable region comprising a sequence of contiguous amino acids that differs from the sequence of a heavy chain variable region in Table 2B, i.e., a VH selected from HV-01 to HV-14, at only 1, 2, 3, 4, 5, 6, 7, 8, 9, 10, 11, 12, 13, 14 or 15 amino acid residues, wherein each such sequence difference is independently either a deletion, insertion or substitution of one amino acid, with the deletions, insertions and/or substitutions resulting in no more than 15 amino acid changes relative to the foregoing variable domain sequences. The heavy chain variable region in some anti-PSMA binding domains comprises a sequence of amino acids that has at least 70%, at least 75%, at least 80%, at least 85%, at least 90%, at least 95%, at least 97% or at least 99% sequence identity to the amino acid sequences of SEQ ID NOs: 33 to 39 or SEQ ID NOs: 170 to 176 (i.e. the heavy chain variable regions in Table 2B).

In one embodiment, the anti-PSMA binding domains of the PSMA-targeted T-cell engaging molecules according to the invention comprise a heavy chain variable region comprising a sequence that is at least 90% identical to a sequence selected from SEQ ID NOs: 33-39 and 170-176. In another embodiment, the anti-PSMA binding domains of the PSMA-targeted T-cell engaging molecules according to the invention comprise a heavy chain variable region comprising a sequence that is at least 95% identical to a sequence selected from SEQ ID NOs: 33-39 and 170-176. In yet another embodiment, the anti-PSMA binding domains of the PSMA-targeted T-cell engaging molecules according to the invention comprise a heavy chain variable region comprising a sequence selected from SEQ ID NOs: 33-39 and 170-176.

The term “identity,” as used herein, refers to a relationship between the sequences of two or more polypeptide molecules or two or more nucleic acid molecules, as determined by aligning and comparing the sequences. “Percent identity.” as used herein, means the percent of identical residues between the amino acids or nucleotides in the compared molecules and is calculated based on the size of the smallest of the molecules being compared. For these calculations, gaps in alignments (if any) must be addressed by a particular mathematical model or computer program (i.e., an “algorithm”). Methods that can be used to calculate the identity of the aligned nucleic acids or polypeptides include those described in Computational Molecular Biology, (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., 1987, Sequence Analysis in Molecular Biology, New York: Academic Press; Sequence Analysis Primer, (Gribskov, M. and Devereux, J., eds.), 1991, New York: M. Stockton Press; and Carillo et al., 1988, SIAM J. Applied Math. 48:1073. For example, sequence identity can be determined by standard methods that are commonly used to compare the similarity in position of the amino acids of two polypeptides. Using a computer program such as BLAST or FASTA, two polypeptide or two polynucleotide sequences are aligned for optimal matching of their respective residues (either along the full length of one or both sequences, or along a pre-determined portion of one or both sequences). The programs provide a default opening penalty and a default gap penalty, and a scoring matrix such as PAM 250 (Dayhoff et al., in Atlas of Protein Sequence and Structure, vol. 5, supp. 3, 1978) or BLOSUM62 (Henikoff et al., 1992, Proc. Natl. Acad. Sci. U.S.A. 89:10915-10919) can be used in conjunction with the computer program. For example, the percent identity can then be calculated as: the total number of identical matches multiplied by 100 and then divided by the sum of the length of the longer sequence within the matched span and the number of gaps introduced into the longer sequences in order to align the two sequences. In calculating percent identity, the sequences being compared are aligned in a way that gives the largest match between the sequences.

The GCG program package is a computer program that can be used to determine percent identity, which package includes GAP (Devereux et al., 1984, Nucl. Acid Res. 12:387; Genetics Computer Group, University of Wisconsin, Madison, WI). The computer algorithm GAP is used to align the two polypeptides or two polynucleotides for which the percent sequence identity is to be determined. The sequences are aligned for optimal matching of their respective amino acid or nucleotide (the “matched span,” as determined by the algorithm). A gap opening penalty (which is calculated as 3× the average diagonal, wherein the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. In certain embodiments, a standard comparison matrix (see, Dayhoff et al., 1978, Atlas of Protein Sequence and Structure 5:345-352 for the PAM 250 comparison matrix; Henikoff et al., 1992, Proc. Natl. Acad. Sci. U.S.A. 89:10915-10919 for the BLOSUM 62 comparison matrix) is also used by the algorithm.

Recommended parameters for determining percent identity for polypeptide or nucleotide sequences using the GAP program include the following:
Algorithm: Needleman et al. 1970, J. Mol. Biol. 48:443-453;
Comparison matrix: BLOSUM 62 from Henikoff et al., 1992, supra;
Gap Penalty: 12 (but with no penalty for end gaps)
Gap Length Penalty: 4
Threshold of Similarity: 0

Certain alignment schemes for aligning two amino acid sequences may result in matching of only a short region of the two sequences, and this small aligned region may have very high sequence identity even though there is no significant relationship between the two full-length sequences. Accordingly, the selected alignment method (GAP program) can be adjusted if so desired to result in an alignment that spans at least 50 contiguous amino acids of the target polypeptide.

The second binding domain of the PSMA-targeted T-cell engaging molecules used in the methods of the invention specifically binds to CD3, preferably human CD3. This binding domain is referred to herein as an anti-CD3 binding domain. “CD3” (cluster of differentiation 3) is a T cell co-receptor composed of four chains. In mammals, the CD3 protein complex contains a CD3γ (gamma) chain, a CD3δ (delta) chain, and two CD3ε (epsilon) chains. These four chains associate with the T cell receptor (TCR) and the so-called ζ (zeta) chain to form the “T cell receptor complex” and to generate an activation signal in T lymphocytes. The CD3γ (gamma), CD3δ (delta), and CD3ε (epsilon) chains are highly related cell-surface proteins of the immunoglobulin superfamily and each contain a single extracellular immunoglobulin domain. The intracellular tails of the CD3 molecules contain a single conserved motif known as an immunoreceptor tyrosine-based activation motif (ITAM), which is essential for the signaling capacity of the TCR. The CD3 epsilon molecule is a polypeptide, which in humans is encoded by the CD3E gene which resides on chromosome 11.

The redirected lysis of target cells via the recruitment of T cells by a T-cell engaging molecule which binds to CD3 on the T cell and to a target protein (e.g. PSMA) on the target cell (e.g. tumor cell) generally involves cytolytic synapse formation and delivery of perforin and granzymes. The engaged T cells are capable of serial target cell lysis, and are not affected by immune escape mechanisms interfering with peptide antigen processing and presentation, or clonal T cell differentiation (see, for example, WO 2007/042261).

In certain embodiments, the second binding domain of the PSMA-targeted T-cell engaging molecules used in the methods of the invention specifically binds to CD3 on the surface of a T cell, more preferably to human CD3 on the surface of a T cell. In some embodiments, the second binding domain of the PSMA-targeted T-cell engaging molecules specifically binds to CD3 epsilon, preferably human CD3 epsilon, e.g. human CD3 epsilon on the surface of a T cell. An exemplary amino acid sequence for the extracellular domain of human CD3 epsilon is provided below as SEQ ID NO: 40:

| (SEQ ID NO: 40) | |
|---|---|
| 1 | QDGNEEMGGI TQTPYKVSIS GTTVILTCPQ YPGSEILWQH NDKNIGGDED DKNIGSDEDH |
| 61 | LSLKEFSELE QSGYYVCYPR GSKPEDANFY LYLRARVCEN CMEMD |

Examples of anti-CD3 binding domains from which the second binding domain of the T-cell engaging molecules used in the methods of the invention can be constructed or derived are described in WO 2007/042261 and WO 2008/119567, both of which are hereby incorporated by reference in their entireties. Light chain and heavy chain variable regions and associated CDRs of exemplary anti-human CD3 antibodies from which the anti-CD3 binding domain of the PSMA-targeted T-cell engaging molecules can be derived or constructed are set forth in Tables 3A and 3B, respectively.

TABLE 3A
Exemplary Anti-Human CD3 Antibody Light Chain Variable Region Amino
Acid Sequences

| Antibody | VL | | | | |
|---|---|---|---|---|---|
| ID. | Group | VL Amino Acid Sequence | CDRL1 | CDRL2 | CDRL3 |
| 101, 102, | LV- | QTVVTQEPSLTVSPGGTVTL | GSSTGAVTSGYY | GTKFLAP | ALWYSNRWV |
| 103, 104, | 101 | TCGSSTGAVTSGYYPNWVQ | PN (SEQ ID NO: | (SEQ ID NO: | (SEQ ID NO: 46) |
| 106,108 | | QKPGQAPRGLIGGTKFLAPG | 41) | 44) | |
| | | TPARFSGSLLGGKAALTLSG | | | |
| | | VQPEDEAEYYCALWYSNRW | | | |
| | | VFGGGTKLTVL (SEQ ID NO: | | | |
| | | 68) | | | |
| 105, 107 | LV- | QTVVTQEPSLTVSPGGTVTL | RSSTGAVTSGYY | ATDMRPS | ALWYSNRWV |
| | 102 | TCRSSTGAVTSGYYPNWVQ | PN (SEQ ID NO: | (SEQ ID NO: | (SEQ ID NO: 46) |
| | | QKPGQAPRGLIGATDMRPSG | 42) | 45) | |
| | | TPARFSGSLLGGKAALTLSG | | | |
| | | VQPEDEAEYYCALWYSNRW | | | |
| | | VFGGGTKLTVL (SEQ ID NO: | | | |
| | | 69) | | | |
| 109, 110 | LV- | QTVVTQEPSLTVSPGGTVTL | GSSTGAVTSGNY | GTKFLAP | VLWYSNRWV |
| | 103 | TCGSSTGAVTSGNYPNWVQ | PN (SEQ ID NO: | (SEQ ID NO: | (SEQ ID NO: 47) |
| | | QKPGQAPRGLIGGTKFLAPG | 43) | 44) | |
| | | TPARFSGSLLGGKAALTLSG | | | |
| | | VQPEDEAEYYCVLWYSNRW | | | |
| | | VFGGGTKLTVL (SEQ ID NO: | | | |
| | | 70) | | | |
| 111 | LV- | EIVMTQSPATLSVSPGERATL | QSVSSN | GAS | QQYNNWPWT |
| | 104 | SCRASQSVSSNLAWYQQKP | (SEQ ID NO: 177) | (SEQ ID NO: | (SEQ ID NO: 179) |
| | | GQAPRLLIYGASTRATGIPAR | | 178) | |
| | | FSGSGSGTEFTLTISSLQSEDF | | | |
| | | AVYYCQQYNNWPWTFGQG | | | |
| | | TKVEIK (SEQ ID NO: 180) | | | |

TABLE 3B
Exemplary Anti-Human CD3 Antibody Heavy Chain Variable Region Amino
Acid Sequences

| Antibody | VH | | | | |
|---|---|---|---|---|---|
| ID. | Group | VH Amino Acid Sequence | CDRH1 | CDRH2 | CDRH3 |
| 101 | HV- | EVQLVESGGGLVQPGGSLK | IYAMN | RIRSKYNN | HGNFGNSYVSFF |
| | 101 | LSCAASGFTFNIYAMNWVR | (SEQ ID NO: 48) | YATYYADS | AY (SEQ ID NO: |
| | | QAPGKGLEWVARIRSKYN | | VKS (SEQ | 59) |
| | | NYATYYADSVKSRFTISRD | | ID NO: 54) | |
| | | DSKNTAYLQMNNLKTEDT | | | |
| | | AVYYCVRHGNFGNSYVSFF | | | |
| | | AYWGQGTLVTVSS (SEQ ID | | | |
| | | NO: 71) | | | |
| 102, 110 | HV- | EVQLVESGGGLVQPGGSLK | KYAMN | RIRSKYNN | HGNFGNSYISY |
| | 102 | LSCAASGFTFNKYAMNWV | (SEQ ID NO: 49) | YATYYADS | WAY (SEQ ID |
| | | RQAPGKGLEWVARIRSKYN | | VKD (SEQ | NO: 60) |
| | | NYATYYADSVKDRFTISRD | | ID NO: 55) | |
| | | DSKNTAYLQMNNLKTEDT | | | |
| | | AVYYCVRHGNFGNSYISY | | | |
| | | WAYWGQGTLVTVSS (SEQ | | | |
| | | ID NO: 72) | | | |
| 103 | HV | EVQLVESGGGLEQPGGSLK | SYAMN | RIRSKYNN | HGNFGNSYLSF |
| | 103 | LSCAASGFTFNSYAMNWV | (SEQ ID NO: 50) | YATYYADS | WAY (SEQ ID |
| | | RQAPGKGLEWVARIRSKYN | | VKG (SEQ | NO: 61) |
| | | NYATYYADSVKGRFTISRD | | ID NO: 56) | |
| | | DSKNTAYLQMNNLKTEDT | | | |
| | | AVYYCVRHGNFGNSYLSF | | | |
| | | WAYWGQGTLVTVSS (SEQ | | | |
| | | ID NO: 73) | | | |
| 104 | HV- | EVQLVESGGGLVQPGGSLK | RYAMN | RIRSKYNN | HGNFGNSYLSYF |
| | 104 | LSCAASGFTFNRYAMNWV | (SEQ ID NO: 51) | YATYYADS | AY (SEQ ID NO: |
| | | RQAPGKGLEWVARIRSKYN | | VKG (SEQ | 62) |
| | | NYATYYADSVKGRFTISRD | | ID NO: 56) | |
| | | DSKNTAYLQMNNLKTEDT | | | |
| | | AVYYCVRHGNFGNSYLSYF | | | |
| | | AYWGQGTLVTVSS (SEQ ID | | | |
| | | NO: 74) | | | |
| 105 | HV- | EVQLVESGGGLVQPGGSLK | VYAMN | RIRSKYNN | HGNFGNSYLSW |
| | 105 | LSCAASGFTFNVYAMNWV | (SEQ ID NO: 52) | YATYYADS | WAY (SEQ ID |
| | | RQAPGKGLEWVARIRSKYN | | VKK | NO: 63) |
| | | NYATYYADSVKKRFTISRD | | (SEQ ID NO: | |
| | | DSKNTAYLQMNNLKTEDT | | 57) | |
| | | AVYYCVRHGNFGNSYLSW | | | |
| | | WAYWGQGTLVTVSS (SEQ | | | |
| | | ID NO: 75) | | | |
| 106 | HV- | EVQLVESGGGLVQPGGSLK | KYAMN | RIRSKYNN | HGNFGNSYTSY |
| | 106 | LSCAASGFTFNKYAMNWV | (SEQ ID NO: 49) | YATYYADS | YAY (SEQ ID NO: |
| | | RQAPGKGLEWVARIRSKYN | | VKS | 64) |
| | | NYATYYADSVKSRFTISRD | | (SEQ ID NO: | |
| | | DSKNTAYLQMNNLKTEDT | | 54) | |
| | | AVYYCVRHGNFGNSYTSY | | | |
| | | YAYWGQGTLVTVSS (SEQ | | | |
| | | ID NO: 76) | | | |
| 107 | HV- | EVQLVESGGGLVQPGGSLK | GYAMN | RIRSKYNN | HRNFGNSYLSW |
| | 107 | LSCAASGFTFNGYAMNWV | (SEQ ID NO: 53) | YATYYADS | FAY (SEQ ID NO: |
| | | RQAPGKGLEWVARIRSKYN | | VKE (SEQ | 65) |
| | | NYATYYADSVKERFTISRD | | ID NO: 58) | |
| | | DSKNTAYLQMNNLKTEDT | | | |
| | | AVYYCVRHRNFGNSYLSW | | | |
| | | FAYWGQGTLVTVSS (SEQ | | | |
| | | ID NO: 77) | | | |
| 108 | HV- | EVQLVESGGGLVQPGGSLK | VYAMN | RIRSKYNN | HGNFGNSYISW |
| | 108 | LSCAASGFTFNVYAMNWV | (SEQ ID NO: 52) | YATYYADS | WAY (SEQ ID |
| | | RQAPGKGLEWVARIRSKYN | | VKK | NO: 66) |
| | | NYATYYADSVKKRFTISRD | | (SEQ ID NO: | |
| | | DSKNTAYLQMNNLKTEDT | | 57) | |
| | | AVYYCVRHGNFGNSYISW | | | |
| | | WAYWGQGTLVTVSS (SEQ | | | |
| | | ID NO: 78) | | | |
| 109 | HV- | EVQLVESGGGLVQPGGSLK | SYAMN | RIRSKYNN | HGNFGNSYVSW |
| | 109 | LSCAASGFTFNSYAMNWV | (SEQ ID NO: 50) | YATYYADS | WAY (SEQ ID |
| | | RQAPGKGLEWVARIRSKYN | | VKG (SEQ | NO: 67) |
| | | NYATYYADSVKGRFTISRD | | ID NO: 56) | |
| | | DSKNTAYLQMNNLKTEDT | | | |
| | | AVYYCVRHGNFGNSYVSW | | | |
| | | WAYWGQGTLVTVSS (SEQ | | | |
| | | ID NO: 79) | | | |
| 111 | HV- | EVQLVESGGGLVQPGRSLR | GFTFDDYA | ISWNSGSI | AKDSRGYGDYR |
| | 110 | LSCAASGFTFDDYAMHWV | (SEQ ID NO: 181) | (SEQ ID NO: | LGGAY |
| | | RQAPGKGLEWVSGISWNSG | | 182) | (SEQ ID NO: 183) |
| | | SIGYADSVKGRFTISRDNAK | | | |
| | | NSLYLQMNSLRAEDTALYY | | | |
| | | CAKDSRGYGDYRLGGAYW | | | |
| | | GQGTLVTVSS (SEQ ID NO: | | | |
| | | 184) | | | |

The domain that specifically binds to human CD3 (e.g., the anti-CD3 binding domain) of the PSMA-targeted T-cell engaging molecules suitable for use in the methods of the invention may comprise one or more of the light chain CDRs (i.e. CDRLs) and/or heavy chain CDRs (i.e. CDRHs) presented in Tables 3A and 3B, respectively. For instance, in some embodiments, the anti-CD3 binding domains of the PSMA-targeted T-cell engaging molecules according to the invention comprise a CDRL1 comprising a sequence selected from SEQ ID NOs: 41 to 43 and 177; a CDRL2 comprising the sequence of SEQ ID NO: 44, 45, or 178; a CDRL3 comprising the sequence of SEQ ID NO: 46, 47, or 179; a CDRH1 comprising a sequence selected from SEQ ID NOs: 48 to 53 and 181; a CDRH2 comprising a sequence selected from SEQ ID NOs: 54 to 58 and 182; and a CDRH3 comprising a sequence selected from SEQ ID NOs: 59 to 67 and 183.

In some embodiments, the anti-CD3 binding domains of the PSMA-targeted T-cell engaging molecules comprise a light chain variable region comprising a CDRL1, a CDRL2, and a CDRL3, wherein: (a) CDRL1, CDRL2, and CDRL3 have the sequence of SEQ ID NOs: 41, 44 and 46, respectively; (b) CDRL1, CDRL2, and CDRL3 have the sequence of SEQ ID NOs: 42, 45 and 46, respectively; (c) CDRL1, CDRL2, and CDRL3 have the sequence of SEQ ID NOs: 43, 44 and 47, respectively; or (d) CDRL1, CDRL2, and CDRL3 have the sequence of SEQ ID NOs: 177, 178 and 179, respectively. In these and other embodiments, the anti-CD3 binding domains of the PSMA-targeted T-cell engaging molecules comprise a heavy chain variable region comprising a CDRH1, a CDRH2, and a CDRH3, wherein: (a) CDRH1, CDRH2, and CDRH3 have the sequence of SEQ ID NOs: 48, 54 and 59, respectively; (b) CDRH1, CDRH2, and CDRH3 have the sequence of SEQ ID NOs: 49, 55 and 60, respectively; (c) CDRH1, CDRH2, and CDRH3 have the sequence of SEQ ID NOs. 50, 56 and 61, respectively; (d) CDRH1, CDRH2, and CDRH3 have the sequence of SEQ ID NOs: 51, 56 and 62, respectively; (e) CDRH1, CDRH2, and CDRH3 have the sequence of SEQ ID NOs: 52, 57 and 63, respectively; (f) CDRH1, CDRH2, and CDRH3 have the sequence of SEQ ID NOs: 49, 54 and 64, respectively; (g) CDRH1, CDRH2, and CDRH3 have the sequence of SEQ ID NOs: 53, 58 and 65, respectively; (h) CDRH1, CDRH2, and CDRH3 have the sequence of SEQ ID NOs: 52, 57 and 66, respectively; (i) CDRH1, CDRH2, and CDRH3 have the sequence of SEQ ID NOs: 50, 56 and 67, respectively; or (j) CDRH1, CDRH2, and CDRH3 have the sequence of SEQ ID NOs: 181, 182, and 183, respectively.

In certain embodiments, the anti-CD3 binding domains of the PSMA-targeted T-cell engaging molecules suitable for use in the methods of the invention comprise a light chain variable region comprising a CDRL1, a CDRL2, and a CDRL3 and a heavy chain variable region comprising a CDRH1, a CDRH2, and a CDRH3, wherein:
(a) CDRL1, CDRL2, and CDRL3 have the sequence of SEQ ID NOs: 41, 44 and 46, respectively, and CDRH1, CDRH2, and CDRH3 have the sequence of SEQ ID NOs: 48, 54 and 59, respectively;
(b) CDRL1, CDRL2, and CDRL3 have the sequence of SEQ ID NOs: 41, 44 and 46, respectively, and CDRH1, CDRH2, and CDRH3 have the sequence of SEQ ID NOs: 49, 55 and 60, respectively;
(c) CDRL1, CDRL2, and CDRL3 have the sequence of SEQ ID NOs: 41, 44 and 46, respectively, and CDRH1, CDRH2, and CDRH3 have the sequence of SEQ ID NOs: 50, 56 and 61, respectively;
(d) CDRL1, CDRL2, and CDRL3 have the sequence of SEQ ID NOs: 41, 44 and 46, respectively, and CDRH1, CDRH2, and CDRH3 have the sequence of SEQ ID NOs: 51, 56 and 62, respectively;
(e) CDRL1, CDRL2, and CDRL3 have the sequence of SEQ ID NOs: 42, 45 and 46, respectively, and CDRH1, CDRH2, and CDRH3 have the sequence of SEQ ID NOs: 52, 57 and 63, respectively;
(f) CDRL1, CDRL2, and CDRL3 have the sequence of SEQ ID NOs: 41, 44 and 46, respectively, and CDRH1, CDRH2, and CDRH3 have the sequence of SEQ ID NOs: 49, 54 and 64, respectively;
(g) CDRL1, CDRL2, and CDRL3 have the sequence of SEQ ID NOs: 42, 45 and 46, respectively, and CDRH1, CDRH2, and CDRH3 have the sequence of SEQ ID NOs: 53, 58 and 65, respectively;
(h) CDRL1, CDRL2, and CDRL3 have the sequence of SEQ ID NOs: 41, 44 and 46, respectively, and CDRH1, CDRH2, and CDRH3 have the sequence of SEQ ID NOs: 52, 57 and 66, respectively;
(i) CDRL1, CDRL2, and CDRL3 have the sequence of SEQ ID NOs: 43, 44 and 47, respectively, and CDRH1, CDRH2, and CDRH3 have the sequence of SEQ ID NOs: 50, 56 and 67, respectively;
(j) CDRL1, CDRL2, and CDRL3 have the sequence of SEQ ID NOs: 43, 44 and 47, respectively, and CDRH1, CDRH2, and CDRH3 have the sequence of SEQ ID NOs: 49, 55 and 60, respectively; or
(k) CDRL1, CDRL2, and CDRL3 have the sequence of SEQ ID NOs: 177, 178, and 179, respectively, and CDRH1, CDRH2, and CDRH3 have the sequence of SEQ ID NOs: 181, 182, and 183, respectively. In a preferred embodiment, the anti-CD3 binding domain of the PSMA-targeted T-cell engaging molecules used in the methods of the invention comprises (i) a light chain variable region comprising a CDRL1 having the sequence of SEQ ID NO: 43, a CDRL2 having the sequence of SEQ ID NO: 44, and a CDRL3 having the sequence of SEQ ID NO: 47, and (ii) a heavy chain variable region comprising a CDRH1 having the sequence of SEQ ID NO: 49, a CDRH2 having the sequence of SEQ ID NO: 55, and a CDRH3 having the sequence of SEQ ID NO. 60. In another preferred embodiment, the anti-CD3 binding domain of the PSMA-targeted T-cell engaging molecules used in the methods of the invention comprises (i) a light chain variable region comprising a CDRL1 having the sequence of SEQ ID NO: 177, a CDRL2 having the sequence of SEQ ID NO: 178, and a CDRL3 having the sequence of SEQ ID NO: 179, and (ii) a heavy chain variable region comprising a CDRH1 having the sequence of SEQ ID NO: 181, a CDRH2 having the sequence of SEQ ID NO: 182, and a CDRH3 having the sequence of SEQ ID NO: 183.

The anti-CD3 binding domain of the PSMA-targeted T-cell engaging molecules according to the invention may comprise a light chain variable region selected from LV-101 to LV-104 (SEQ ID NOs: 68-70 and 180), as shown in Table 3A, and/or a heavy chain variable region selected from HV-101 to HV-110 (SEQ ID NOs: 71-79 and 184), as shown in Table 3B, and binding fragments, derivatives, and variants of these light chain and heavy chain variable regions. Each of the light chain variable regions listed in Table 3A may be combined with any of the heavy chain variable regions listed in Table 3B to form an anti-CD3 binding domain of the PSMA-targeted T-cell engaging molecules according to the invention. Examples of such combinations include, but are not limited to: (i) LV-101 and HV-101; (ii) LV-101 and HV-102; (iii) LV-101 and HV-103; (iv) LV-101 and HV-104; (v) LV-101 and HV-106; (vi) LV-101 and HV-108; (vii) LV-102 and HV-105; (viii) LV-102 and HV-107; (ix) LV-103 and HV-109; (x) LV-103 and HV-102; and (xi) LV-104 and HV-110.

In certain embodiments, the anti-CD3 binding domains of the PSMA-targeted T-cell engaging molecules according to the invention comprise a light chain variable region comprising the sequence of SEQ ID NO: 68 and a heavy chain variable region comprising the sequence of SEQ ID NO: 71. In some embodiments, the anti-CD3 binding domains of the PSMA-targeted T-cell engaging molecules according to the invention comprise a light chain variable region comprising the sequence of SEQ ID NO: 68 and a heavy chain variable region comprising the sequence of SEQ ID NO: 72. In other embodiments, the anti-CD3 binding domains of the PSMA-targeted T-cell engaging molecules according to the invention comprise a light chain variable region comprising the sequence of SEQ ID NO: 68 and a heavy chain variable region comprising the sequence of SEQ ID NO: 73. In still other embodiments, the anti-CD3 binding domains of the PSMA-targeted T-cell engaging molecules according to the invention comprise a light chain variable region comprising the sequence of SEQ ID NO: 68 and a heavy chain variable region comprising the sequence of SEQ ID NO: 74. In some embodiments, the anti-CD3 binding domains of the PSMA-targeted T-cell engaging molecules according to the invention comprise a light chain variable region comprising the sequence of SEQ ID NO: 69 and a heavy chain variable region comprising the sequence of SEQ ID NO: 75. In certain embodiments, the anti-CD3 binding domains of the PSMA-targeted T-cell engaging molecules according to the invention comprise a light chain variable region comprising the sequence of SEQ ID NO: 68 and a heavy chain variable region comprising the sequence of SEQ ID NO: 76. In one embodiment, the anti-CD3 binding domains of the PSMA-targeted T-cell engaging molecules according to the invention comprise a light chain variable region comprising the sequence of SEQ ID NO: 69 and a heavy chain variable region comprising the sequence of SEQ ID NO: 77. In another embodiment, the anti-CD3 binding domains of the PSMA-targeted T-cell engaging molecules according to the invention comprise a light chain variable region comprising the sequence of SEQ ID NO: 68 and a heavy chain variable region comprising the sequence of SEQ ID NO: 78. In a preferred embodiment, the anti-CD3 binding domains of the PSMA-targeted T-cell engaging molecules according to the invention comprise a light chain variable region comprising the sequence of SEQ ID NO: 70 and a heavy chain variable region comprising the sequence of SEQ ID NO: 72. In another preferred embodiment, the anti-CD3 binding domains of the PSMA-targeted T-cell engaging molecules according to the invention comprise a light chain variable region comprising the sequence of SEQ ID NO: 70 and a heavy chain variable region comprising the sequence of SEQ ID NO: 79. In still another embodiment, the anti-CD3 binding domains of the PSMA-targeted T-cell engaging molecules according to the invention comprise a light chain variable region comprising the sequence of SEQ ID NO: 180 and a heavy chain variable region comprising the sequence of SEQ ID NO: 184.

In some embodiments, the anti-CD3 binding domains of the PSMA-targeted T-cell engaging molecules according to the invention comprise a light chain variable region comprising a sequence of contiguous amino acids that differs from the sequence of a light chain variable region in Table 3A, i.e. a VL selected from LV-101 to LV-104, at only 1, 2, 3, 4, 5, 6, 7, 8, 9, 10, 11, 12, 13, 14 or 15 amino acid residues, wherein each such sequence difference is independently either a deletion, insertion or substitution of one amino acid, with the deletions, insertions and/or substitutions resulting in no more than 15 amino acid changes relative to the foregoing variable domain sequences. The light chain variable region in some anti-CD3 binding domains comprises a sequence of amino acids that has at least 70%, at least 75%, at least 80%, at least 85%, at least 90%, at least 95%, at least 97% or at least 99% sequence identity to the amino acid sequences of SEQ ID NOs. 68 to 70 and 180 (i.e. the light chain variable regions in Table 3A).

In one embodiment, the anti-CD3 binding domains of the PSMA-targeted T-cell engaging molecules according to the invention comprise a light chain variable region comprising a sequence that is at least 90% identical to a sequence selected from SEQ ID NOs: 68-70 and 180. In another embodiment, the anti-CD3 binding domains of the PSMA-targeted T-cell engaging molecules according to the invention comprise a light chain variable region comprising a sequence that is at least 95% identical to a sequence selected from SEQ ID NOs: 68-70 and 180. In yet another embodiment, the anti-CD3 binding domains of the PSMA-targeted T-cell engaging molecules according to the invention comprise a light chain variable region comprising a sequence selected from SEQ ID NOs: 68-70 and 180.

In these and other embodiments, the anti-CD3 binding domains of the PSMA-targeted T-cell engaging molecules according to the invention comprise a heavy chain variable region comprising a sequence of contiguous amino acids that differs from the sequence of a heavy chain variable region in Table 3B, i.e., a VH selected from HV-101 to HV-110, at only 1, 2, 3, 4, 5, 6, 7, 8, 9, 10, 11, 12, 13, 14 or 15 amino acid residues, wherein each such sequence difference is independently either a deletion, insertion or substitution of one amino acid, with the deletions, insertions and/or substitutions resulting in no more than 15 amino acid changes relative to the foregoing variable domain sequences. The heavy chain variable region in some anti-CD3 binding domains comprises a sequence of amino acids that bas at least 70%, at least 75%, at least 80%, at least 85%, at least 90%, at least 95%, at least 97% or at least 99% sequence identity to the amino acid sequences of SEQ ID NOs: 71 to 79 and 184 (i.e. the heavy chain variable regions in Table 3B).

In one embodiment, the anti-CD3 binding domains of the PSMA-targeted T-cell engaging molecules according to the invention comprise a heavy chain variable region comprising a sequence that is at least 90% identical to a sequence selected from SEQ ID NOs: 71-79 and 184. In another embodiment, the anti-CD3 binding domains of the PSMA-targeted T-cell engaging molecules according to the invention comprise a heavy chain variable region comprising a sequence that is at least 95% identical to a sequence selected from SEQ ID NOs: 71-79 and 184. In yet another embodiment, the anti-CD3 binding domains of the PSMA-targeted T-cell engaging molecules according to the invention comprise a heavy chain variable region comprising a sequence selected from SEQ ID NOs: 71-79 and 184.

According to certain embodiments, one or more of the binding domains of the PSMA-targeted T-cell engaging molecule used in the methods of the invention, are in the format of an scFv. In an scFv, the VH region and the VL region are arranged in the order VH-VL or VL-VH (from N- to C-terminus). It is envisaged that the VH and the VL regions of the first and/or the second binding domain are connected via a linker, preferably a peptide linker. In one embodiment of the first and/or second binding domain, the VH-region is positioned N-terminally of the linker, and the VL-region is positioned C-terminally of the linker. The linkers are preferably peptide linkers, more preferably short peptide linkers. Examples of suitable linkers include, but are not limited to:

(SEQ ID NO: 80)
GGGG
(SEQ ID NO: 81)
GGGGS
(SEQ ID NO: 82)
GGGGSGGGGS
(SEQ ID NO: 83)
GGGGSGGGGSGGGGS
(SEQ ID NO: 84)
GGGGSGGGGSGGGGSGGGGS
(SEQ ID NO: 85)
GGGGSGGGGSGGGGSGGGGGGGGS
(SEQ ID NO: 86)
GGGGSGGGGSGGGGSGGGGSGGGGSGGGGS
(SEQ ID NO: 87)
GGGGSGGGGSGGGGSGGGGSGGGGSGGGGSGGGGS
(SEQ ID NO: 88)
GGGGGGGGSGGGGSGGGGSGGGGSGGGGSGGGGSGGGGS
(SEQ ID NO: 89)
PGGGGS
(SEQ ID NO: 90)
PGGDGS
(SEQ ID NO: 91)
SGGGGS
(SEQ ID NO: 92)
GGGGSGGGS
(SEQ ID NO: 93)
GGGGQ

In the present context, a “short” linker has between 2 and 50 amino acids, preferably between 3 and 35, between 4 and 30, between 5 and 25, between 6 and 20 or between 6 and 17 amino acids. The linker between two variable regions of one binding domain may have a different length (e.g. may be longer) than the linker between the two binding domains. For example, the linker between two variable regions of one or both binding domains may have a length between 8 and 16 amino acids, preferably between 10 and 15, and the linker between the two binding domains may have a length between 3 and 10 amino acids, preferably between S and 8. It is further envisaged that the peptide linkers are glycine/serine linkers, such as those depicted in SEQ ID NOs: 81-92. In one embodiment, the anti-PSMA binding domain and/or the anti-CD3 binding domain of the PSMA-targeted T-cell engaging molecule according to the invention is an scFv comprising, from N-terminus to C-terminus, a VH region-peptide linker-VL region, where the peptide linker comprises a glycine-serine linker, such as the linker set forth in SEQ ID NO: 83. In related embodiments, the peptide linker between the anti-PSMA and anti-CD3 binding domains (e.g. scFv domains) is the linker set forth in SEQ ID NO: 81 or SEQ ID NO: 91. Exemplary scFv domains for the anti-PSMA and anti-CD3 binding domains of the PSMA-targeted T-cell engaging molecules suitable for use in the methods of the invention are set forth in Table 4 below.

TABLE 4
Exemplary Single-Chain Variable Fragment Binding Domains

| | | |
|---|---|---|
| | SEQ ID | |
| Designation | NO. | Amino Acid Sequence |

Anti-PSMA scFv domains

| | | |
|---|---|---|
| PSMA scFv-01 | 94 | QVQLVESGGGLVKPGESLRLSCAASGFTFSDYYMYWVRQAPGKCLEWVAIISD |
| | | GGYYTYYSDIIKGRFTISRDNAKNSLYLQMNSLKAEDTAVYYCARGFPLLRHG |
| | | AMDYWGQGTLVTVSSGGGGSGGGGSGGGGSDIQMTQSPSSLSASVGDRVTITC |
| | | KASQNVDTNVAWYQQKPGQAPKSLIYSASYRYSDVPSRFSGSASGTDFTLTISS |
| | | VQSEDFATYYCQQYDQQLITFGCGTKLEIK |
| PSMA scFv-02 | 95 | QVQLVESGGGLVKPGESLRLSCAASGFTFSDYYMYWVRQAPGKCLEWVAIISD |
| | | GGYYTYYSDIIKGRFTISRDNAKNSLYLQMNSLKAEDTAVYYCARGFPLLRHG |
| | | AMDYWGQGTLVTVSSGGGGSGGGGSGGGGSDIQMTQSPSSLSASVGDRVTITC |
| | | KASQNVDTNVAWYQQKPGQAPKSLIYSASYRYSDVPSRFSGSASGTDFTLTISS |
| | | VQSEDFATYYCQQYDQVRLTFGCGTKLEIK |
| PSMA scFv-03 | 96 | QVQLVESGGGLVKPGESLRLSCAASGFTFSDYYMYWVRQAPGKCLEWVAIISD |
| | | HHHWTYYSDIIKGRFTISRDNAKNSLYLQMNSLKAEDTAVYYCARGFPLLRHG |
| | | AMDYWGQGTLVTVSSGGGGSGGGGSGGGGSDIQMTQSPSSLSASVGDRVTITC |
| | | KASQNVDTNVAWYQQKPGQAPKSLIYSASYRYSDVPSRFSGSASGTDFTLTISS |
| | | VQSEDFATYYCQQYDSYPYTFGCGTKLEIK |
| PSMA scFv-04 | 97 | QVQLVESGGGLVKPGESLRLSCAASGFTFSDYYMYWVRQAPGKCLEWVAIISD |
| | | GGYYTYYSDIIKGRFTISRDNAKNSLYLQMNSLKAEDTAVYYCARGFPLLRHG |
| | | AMDYWGQGTLVTVSSGGGGSGGGGSGGGGSDIQMTQSPSSLSASVGDRVTITC |
| | | KASQNVDTNVAWYQQKPGQAPKSLIYSASYRYSDVPSRFSGSASGTDFTLTISS |
| | | VQSEDFATYYCQQYDRQPLTFGCGTKLEIK |
| PSMA scFv-05 | 98 | QVQLVESGGGLVKPGESLRLSCAASGFTFSDYYMYWVRQAPGKCLEWVAIISD |
| | | GGYYTYYSDIIKGRFTISRDNAKNSLYLQMNSLKAEDTAVYYCARGFPLLRHG |
| | | AMDYWGQGTLVTVSSGGGGSGGGGSGGGGSDIQMTQSPSSLSASVGDRVTITC |
| | | KASQNVDTNVAWYQQKPGQAPKSLIYSASYRYSDVPSRFSGSASGTDFTLTISS |
| | | VQSEDFATYYCQQYDAPMGTFGCGTKLEIK |
| PSMA scFv-06 | 99 | QVQLVESGGGLVKPGESLRLSCAASGFTFSDYYMNWVRQAPGKGLEWVAIISD |
| | | NKHQTYYSDIIKGRFAISRDNAKNSLYLQMNSLKAEDTAVYYCARGFPLLRHG |
| | | AMDYWGQGTLVTVSSGGGGSGGGGSGGGGSDIQMTQSPSSLSASVGDRVTITC |
| | | KASQNVDTNVAWYQQKPGQAPKSLIYSASYRYSDVPSRFSGSASGTDFTLTISS |
| | | LQSEDFATYYCQQYDSYPYTFGGGTKLEIK |
| PSMA scFv-07 | 100 | QVQLVESGGGLVKPGESLRLSCAASGFTFSDYYMNWVRQAPGKCLEWVAIISD |
| | | NKHQTYYSDIIKGRFAISRDNAKNSLYLQMNSLKAEDTAVYYCARGFPLLRHG |
| | | AMDYWGQGTLVTVSSGGGGSGGGGSGGGGSDIQMTQSPSSLSASVGDRVTITC |
| | | KASQNVDTNVAWYQQKPGQAPKSLIYSASYRYSDVPSRFSGSASGTDFTLTISS |
| | | LQSEDFATYYCQQYDSYPYTFGCGTKLEIK |
| PSMA scFv-08 | 101 | QVQLVESGGGLVKPGESLRLSCAASGFTFSDYYMYWVRQAPGKGLEWVAIISD |
| | | GGYYTYYSDIIKGRFTISRDNAKNSLYLQMNSLKAEDTAVYYCARGFPLLRHG |
| | | AMDYWGQGTLVTVSSGGGGSGGGGSGGGGSDIQMTQSPSSLSASVGDRVTITC |
| | | KASQNVDTNVAWYQQKPGQAPKSLIYSASYRYSDVPSRFSGSASGTDFTLTISS |
| | | VQSEDFATYYCQQYDSYPYTFGGGTKLEIK |
| PSMA scFv-09 | 102 | QVQLVESGGGLVKPGESLRLSCAASGFTFSDYYMYWVRQAPGKCLEWVAIISD |
| | | GGYYTYYSDIIKGRFTISRDNAKNSLYLQMNSLKAEDTAVYYCARGFPLLRHG |
| | | AMDYWGQGTLVTVSSGGGGSGGGGSGGGGSDIQMTQSPSSLSASVGDRVTITC |
| | | KASQNVDTNVAWYQQKPGQAPKSLIYSASYRYSDVPSRFSGSASGTDFTLTISS |
| | | VQSEDFATYYCQQYDSYPYTFGCGTKLEIK |
| PSMA scFv-10 | 103 | QVQLVESGGGLVKPGESLRLSCAASGFTFSDYYMYWVRQAPGKGLEWVAIISD |
| | | GGYYTYYSDIIKGRFTISRDNAKNSLYLQMNSLKAEDTAVYYCARGFPLLRHG |
| | | AMDYWGQGTLVTVSSGGGGSGGGGSGGGGSDIQMTQSPSSLSASVGDRVTITC |
| | | KASQNVDTNVAWYQQKPGQAPKSLIYSASYVYWDVPSRFSGSASGTDFTLTIS |
| | | SVQSEDFATYYCQQYDQQLITFGGGTKLEIK |
| PSMA scFv-11 | 104 | QVQLVESGGGLVKPGESLRLSCAASGFTFSDYYMYWVRQAPGKCLEWVAIISD |
| | | GGYYTYYSDIIKGRFTISRDNAKNSLYLQMNSLKAEDTAVYYCARGFPLLRHG |
| | | AMDYWGQGTLVTVSSGGGGSGGGGSGGGGSDIQMTQSPSSLSASVGDRVTITC |
| | | KASQNVDTNVAWYQQKPGQAPKSLIYSASYVYWDVPSRFSGSASGTDFTLTIS |
| | | SVQSEDFATYYCQQYDQQLITFGCGTKLEIK |
| PSMA scFv-12 | 105 | QVQLVESGGGLVKPGESLRLSCAASGFTFSDYYMYWVRQAPGKGLEWVAIISD |
| | | AGYYTYYSDIIKGRFTISRDNAKNSLYLQMNSLKAEDTAVYYCARGFPLLRHG |
| | | AMDYWGQGTLVTVSSGGGGSGGGGSGGGGSDIQMTQSPSSLSASVGDRVTITC |
| | | KASQNVDANVAWYQQKPGQAPKSLIYSASYVYWDVPSRFSGSASGTDFTLTIS |
| | | SVQSEDFATYYCQQYDQQLITFGGGTKLEIK |
| PSMA scFv-13 | 106 | QVQLVESGGGLVKPGESLRLSCAASGFTFSDYYMYWVRQAPGKCLEWVAIISD |
| | | AGYYTYYSDIIKGRFTISRDNAKNSLYLQMNSLKAEDTAVYYCARGFPLLRHG |
| | | AMDYWGQGTLVTVSSGGGGSGGGGSGGGGSDIQMTQSPSSLSASVGDRVTITC |
| | | KASQNVDANVAWYQQKPGQAPKSLIYSASYVYWDVPSRFSGSASGTDFTLTIS |
| | | SVQSEDFATYYCQQYDQQLITFGCGTKLEIK |

Anti-CD3 scFv domains

| | | |
|---|---|---|
| CD3 scFv-01 | 107 | EVQLVESGGGLVQPGGSLKLSCAASGFTFNIYAMNWVRQAPGKGLEWVARIR |
| | | SKYNNYATYYADSVKSRFTISRDDSKNTAYLQMNNLKTEDTAVYYCVRHGNF |
| | | GNSYVSFFAYWGQGTLVTVSSGGGGSGGGGSGGGGSQTVVTQEPSLTVSPGGT |
| | | VTLTCGSSTGAVTSGYYPNWVQQKPGQAPRGLIGGTKFLAPGTPARFSGSLLG |
| | | GKAALTLSGVQPEDEAEYYCALWYSNRWVFGGGTKLTVL |
| CD3 scFv-02 | 108 | EVQLVESGGGLVQPGGSLKLSCAASGFTFNKYAMNWVRQAPGKGLEWVARIR |
| | | SKYNNYATYYADSVKDRFTISRDDSKNTAYLQMNNLKTEDTAVYYCVRHGNF |
| | | GNSYISYWAYWGQGTLVTVSSGGGGSGGGGSGGGGSQTVVTQEPSLTVSPGG |
| | | TVTLTCGSSTGAVTSGYYPNWVQQKPGQAPRGLIGGTKFLAPGTPARFSGSLLG |
| | | GKAALTLSGVQPEDEAEYYCALWYSNRWVFGGGTKLTVL |
| CD3 scFv-03 | 109 | EVQLVESGGGLEQPGGSLKLSCAASGFTFNSYAMNWVRQAPGKGLEWVARIR |
| | | SKYNNYATYYADSVKGRFTISRDDSKNTAYLQMNNLKTEDTAVYYCVRHGNF |
| | | GNSYLSFWAYWGQGTLVTVSSGGGGSGGGGSGGGGSQTVVTQEPSLTVSPGG |
| | | TVTLTCGSSTGAVTSGYYPNWVQQKPGQAPRGLIGGTKFLAPGTPARFSGSLLG |
| | | GKAALTLSGVQPEDEAEYYCALWYSNRWVFGGGTKLTVL |
| CD3 scFv-04 | 110 | EVQLVESGGGLVQPGGSLKLSCAASGFTFNRYAMNWVRQAPGKGLEWVARIR |
| | | SKYNNYATYYADSVKGRFTISRDDSKNTAYLQMNNLKTEDTAVYYCVRHGNF |
| | | GNSYLSYFAYWGQGTLVTVSSGGGGSGGGGSGGGGSQTVVTQEPSLTVSPGG |
| | | TVTLTCGSSTGAVTSGYYPNWVQQKPGQAPRGLIGGTKFLAPGTPARFSGSLLG |
| | | GKAALTLSGVQPEDEAEYYCALWYSNRWVFGGGTKLTVL |
| CD3 scFv-05 | 111 | EVQLVESGGGLVQPGGSLKLSCAASGFTFNVYAMNWVRQAPGKGLEWVARIR |
| | | SKYNNYATYYADSVKKRFTISRDDSKNTAYLQMNNLKTEDTAVYYCVRHGNF |
| | | GNSYLSWWAYWGQGTLVTVSSGGGGSGGGGSGGGGSQTVVTQEPSLTVSPG |
| | | GTVTLTCRSSTGAVTSGYYPNWVQQKPGQAPRGLIGATDMRPSGTPARFSGSL |
| | | LGGKAALTLSGVQPEDEAEYYCALWYSNRWVFGGGTKLTVL |
| CD3 scFv-06 | 112 | EVQLVESGGGLVQPGGSLKLSCAASGFTFNKYAMNWVRQAPGKGLEWVARIR |
| | | SKYNNYATYYADSVKSRFTISRDDSKNTAYLQMNNLKTEDTAVYYCVRHGNF |
| | | GNSYTSYYAYWGQGTLVTVSSGGGGSGGGGSGGGGSQTVVTQEPSLTVSPGG |
| | | TVTLTCGSSTGAVTSGYYPNWVQQKPGQAPRGLIGGTKFLAPGTPARFSGSLLG |
| | | GKAALTLSGVQPEDEAEYYCALWYSNRWVFGGGTKLTVL |
| CD3 scFv-07 | 113 | EVQLVESGGGLVQPGGSLKLSCAASGFTFNGYAMNWVRQAPGKGLEWVARIR |
| | | SKYNNYATYYADSVKERFTISRDDSKNTAYLQMNNLKTEDTAVYYCVRHRNF |
| | | GNSYLSWFAYWGQGTLVTVSSGGGGSGGGGSGGGGSQTVVTQEPSLTVSPGG |
| | | TVTLTCRSSTGAVTSGYYPNWVQQKPGQAPRGLIGATDMRPSGTPARFSGSLL |
| | | GGKAALTLSGVQPEDEAEYYCALWYSNRWVFGGGTKLTVL |
| CD3 scFv-08 | 114 | EVQLVESGGGLVQPGGSLKLSCAASGFTFNVYAMNWVRQAPGKGLEWVARIR |
| | | SKYNNYATYYADSVKKRFTISRDDSKNTAYLQMNNLKTEDTAVYYCVRHGNF |
| | | GNSYISWWAYWGQGTLVTVSSGGGGSGGGGSGGGGSQTVVTQEPSLTVSPGG |
| | | TVTLTCGSSTGAVTSGYYPNWVQQKPGQAPRGLIGGTKFLAPGTPARFSGSLLG |
| | | GKAALTLSGVQPEDEAEYYCALWYSNRWVFGGGTKLTVL |
| CD3 scFv-09 | 115 | EVQLVESGGGLVQPGGSLKLSCAASGFTFNSYAMNWVRQAPGKGLEWVARIR |
| | | SKYNNYATYYADSVKGRFTISRDDSKNTAYLQMNNLKTEDTAVYYCVRHGNF |
| | | GNSYVSWWAYWGQGTLVTVSSGGGGSGGGGSGGGGSQTVVTQEPSLTVSPG |
| | | GTVTLTCGSSTGAVTSGNYPNWVQQKPGQAPRGLIGGTKFLAPGTPARFSGSLL |
| | | GGKAALTLSGVQPEDEAEYYCVLWYSNRWVFGGGTKLTVL |
| CD3 scFv-10 | 116 | EVQLVESGGGLVQPGGSLKLSCAASGFTFNKYAMNWVRQAPGKGLEWVARIR |
| | | SKYNNYATYYADSVKDRFTISRDDSKNTAYLQMNNLKTEDTAVYYCVRHGNF |
| | | GNSYISYWAYWGQGTLVTVSSGGGGSGGGGSGGGGSQTVVTQEPSLTVSPGG |
| | | TVTLTCGSSTGAVTSGNYPNWVQQKPGQAPRGLIGGTKFLAPGTPARFSGSLLG |
| | | GKAALTLSGVQPEDEAEYYCVLWYSNRWVFGGGTKLTVL |

In certain embodiments, the PSMA-targeted T-cell engaging molecules suitable for use in the methods of the invention comprise a first binding domain that specifically binds to human PSMA and has an amino acid sequence selected from any one of SEQ ID NOs: 94-106, and a second binding domain that specifically binds to human CD3 and has an amino acid sequence selected from any one of SEQ ID NOs: 107-116. In a preferred embodiment, the first binding domain (e.g. anti-PSMA binding domain) of the PSMA-targeted T-cell engaging molecules comprises the amino acid sequence of SEQ ID NO: 104. In another preferred embodiment, the second binding domain (e.g. the anti-CD3 binding domain) of the PSMA-targeted T-cell engaging molecules comprises the amino acid sequence of SEQ ID NO: 116.

The PSMA-targeted T-cell engaging molecules according to the invention can comprise any of the anti-PSMA scFv binding domains set forth in Table 4 in combination with any of the anti-CD3 scFv binding domains set forth in Table 4. For instance, in some embodiments, the PSMA-targeted T-cell engaging molecules comprise an anti-PSMA scFv binding domain from Table 4 and an anti-CD3 scFv binding domain from Table 4, wherein the anti-PSMA scFv binding domain is connected to the anti-CD3 scFv binding domain through a peptide linker, such as the peptide linkers described herein. In certain embodiments, the PSMA-targeted T-cell engaging molecule comprises, in amino to carboxyl order, an anti-PSMA scFv binding domain, a peptide linker, and an anti-CD3 scFv binding domain. In some such embodiments, the peptide linker comprises the sequence of SEQ ID NO. 81 or SEQ ID NO: 91.

The PSMA-targeted T-cell engaging molecules according to the invention may also comprise additional domains, which, e.g., can modulate the pharmacokinetic profile of the molecule. For instance, the PSMA-targeted T-cell engaging molecules may further comprise an immunoglobulin Fc domain, a domain derived from serum albumin (e.g. human serum albumin), or an albumin-binding domain (e.g. comprising human albumin binding peptides), and/or be conjugated to polyethylene glycol chains to increase the serum half-life of the T-cell engaging molecule. In certain embodiments, the PSMA-targeted T-cell engaging molecules used in the methods of the invention further comprise one or more immunoglobulin Fc domains. Each immunoglobulin Fc domain may comprise one or more Fc monomers Each “Fc monomer” typically comprises at least a CH2 domain and a CH3 domain from an immunoglobulin molecule. The Fc monomer may comprise the CH2 and CH3 domains from an IgG1, IgG2, IgG3, or IgG4 immunoglobulin. As an example, the CH2 domain comprises amino acids 231 to 340 of an IgG1 immunoglobulin and the CH3 domain comprises amino acids 341 to 446 of an IgG1 immunoglobulin, where the amino acid numbering is according to the EU numbering system described in Edelman et al., Proc. Natl. Acad. USA, Vol. 63:78-85 (1969) and Kabat et al., Sequences of Proteins of Immunological Interest, 5th Ed. Public Health Service, National Institutes of Health Publication No. 91-3242, Bethesda, MD (1991). The boundaries of the CH2 and CH3 domains may vary slightly from one IgG isoform to another, but the CH2 and CH3 domains in IgG2, IgG3, and IgG4 can be ascertained by alignment with the CH2 and CH3 domains in IgG1.

In some embodiments, the Fc monomer may comprise an immunoglobulin hinge region or portion thereof. The immunoglobulin hinge region is typically the region defined by amino acids 216 to 231 (according to the EU numbering system) of IgG immunoglobulins. In certain embodiments, the Fc monomer comprises a hinge region from an IgG1 immunoglobulin or a portion thereof. In some such embodiments, the IgG1 hinge region comprises the amino acid sequence DKTHTCPPCP (SEQ ID NO: 117) or EPKSCDKTHTCPPCP (SEQ ID NO: 118). In other embodiments, the Fc monomer comprises an IgG2 hinge region having the sequence ERKCCVECPPCP (SEQ ID NO: 119), an IgG3 hinge region having the sequence ELKTPLDTTHTCPRCP (SEQ ID NO: 120), EPKSCDTPPPCPRCP (SEQ ID NO: 121), or ELKTPLGDTTHTCPRCP (SEQ ID NO: 122), or an IgG4 hinge region having the sequence ESKYGPPCPSCP (SEQ ID NO: 123). In certain embodiments, the Fe monomer comprises, in amino to carboxyl order, an immunoglobulin hinge region, an immunoglobulin CH2 domain, and an immunoglobulin CH3 domain.

In certain embodiments, the PSMA-targeted T-cell engaging molecules comprise an Fc domain having one Fc monomer. In alternative embodiments, the PSMA-targeted T-cell engaging molecules comprise an Fc domain having two or more Fc monomers. For instance, in one embodiment, the PSMA-targeted T-cell engaging molecules used in the methods of the invention comprise an Fc domain having two Fc monomers. The two Fc monomers can be present on separate polypeptide chains and associate to form a dimer, e.g. via non-covalent interactions and/or disulfide bonds (e.g. between cysteine residues in the hinge regions of Fc monomers). In a preferred embodiment, the two Fc monomers are fused to each other via a peptide linker, preferably a linker sufficient in length to allow the Fc monomers to associate and form an intra-chain dimer. The fusion of two Fc monomers to form a single polypeptide chain is referred to herein as a single-chain Fc domain (scFc domain) and is described in more detail below.

The peptide linker, by which the Fc monomers are fused to each other to form a single-chain Fc domain, preferably comprises at least 25 amino acid residues (e.g. 25, 26, 27, 28, 29, 30 or more). More preferably, this peptide linker comprises at least 30 amino acid residues (e.g. 30, 31, 32, 33, 34, 35 or more). In some embodiments, the linker comprises up to 40 amino acid residues, more preferably up to 35 amino acid residues, and even more preferably exactly 30 amino acid residues. In certain embodiments, the peptide linker comprises glycine-serine residues, for example repeats of the amino acid sequence Gly-Gly-Gly-Gly-Ser (SEQ ID NO: 81). In such embodiments, the peptide linker comprises (Gly₄Ser)ₓ, where x is an integer of 5 or greater (e.g. 6, 7 or 8). Preferably the integer is 6 or 7, more preferably the integer is 6. In one particular embodiment, the peptide linker used to connect the two Fc monomers to form a single-chain Fc domain comprises the sequence of SEQ ID NO: 86.

The Fc monomer may contain one or more amino acid substitutions relative to the native CH2 or CH3 immunoglobulin amino acid sequences, e.g. to modulate effector function, alter glycosylation, or enhance stability. For instance, in one embodiment, the glycosylation site in the CH2 domain at amino acid position 297 according to EU numbering is removed by substituting a different amino acid for the asparagine residue at this position. A N297G substitution is preferred in some embodiments. Stability-enhancing mutations include the substitution of one or more amino acids in the CH2 and/or CH3 domains with cysteine residues to promote disulfide bond formation. Preferably, specific pairs of residues are substituted with cysteine such that they preferentially form a disulfide bond with each other, thus limiting or preventing disulfide bond scrambling. Preferred pairs include, but are not limited to, A287C and L306C, V259C and L306C, R292C and V302C, and V323C and 1332C, with the amino acid positions numbered according to the EU numbering system. In one particular embodiment, the Fc monomer(s) incorporated into the Fc domain of the PSMA-targeted T-cell engaging molecules comprises N297G, R292C, and V302C substitutions, with the amino acid positions numbered according to the EU numbering system.

In certain preferred embodiments, the PSMA-targeted T-cell engaging molecules used in the methods of the invention comprise an Fc domain, which is a single-chain Fc domain. Accordingly, in certain such embodiments, the Fc domain comprises two Fc monomers, each monomer comprising an immunoglobulin hinge region, an immunoglobulin CH2 domain, and an immunoglobulin CH3 domain, wherein the two Fc monomers are fused to each other via a peptide linker as described herein. Exemplary amino acid sequences for the Fe monomers and the single-chain Fc (scFc) domains are provided in Table 5 below. In some embodiments, each of the Fc monomers of the Fc domain has an amino acid sequence that is at least 90% identical to a sequence selected from SEQ ID NOs: 124-131. In other embodiments, each of the Fc monomers of the Fc domain has an amino acid sequence selected from SEQ ID NOs: 124-131. In a preferred embodiment, each of the Fc monomers of the Fc domain comprises the amino acid sequence of SEQ ID NO: 124. In another preferred embodiment, each of the Fc monomers of the Fc domain comprises the amino acid sequence of SEQ ID NO: 125.

TABLE 5
Exemplary Fc Monomer and Single-Chain Fc Domains

| | | |
|---|---|---|
| | SEQ | |
| Designation | ID NO. | Amino Acid Sequence |

Fc Monomers

| | | |
|---|---|---|
| Fc monomer-1 | 124 | DKTHTCPPCPAPELLGGPSVFLFPPKPKDTLMISRTPEVTCVVVDVSHEDPE |
| | | VKFNWYVDGVEVHNAKTKPCEEQYGSTYRCVSVLTVLHQDWLNGKEYK |
| | | CKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGN |
| | | VFSCSVMHEALHNHYTQKSLSLSPGK |
| Fc monomer-2 | 125 | DKTHTCPPCPAPELLGGPSVFLFPPKPKDTLMISRTPEVTCVVVDVSHEDPE |
| | | VKFNWYVDGVEVHNAKTKPCEEQYGSTYRCVSVLTVLHQDWLNGKEYK |
| | | CKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGN |
| | | VFSCSVMHEALHNHYTQKSLSLSP |
| Fc monomer-3 | 126 | DKTHTCPPCPAPELLGGPSVFLFPPKPKDTLMISRTPEVTCVVVDVSHEDPE |
| | | VKFNWYVDGVEVHNAKTKPREEQYNSTYRVVSVLTVLHQDWLNGKEYK |
| | | CKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGN |
| | | VFSCSVMHEALHNHYTQKSLSLSPGK |
| Fc monomer-4 | 127 | DKTHTCPPCPAPELLGGPSVFLFPPKPKDTLMISRTPEVTCVVVDVSHEDPE |
| | | VKFNWYVDGVEVHNAKTKPREEQYNSTYRVVSVLTVLHQDWLNGKEYK |
| | | CKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGN |
| | | VFSCSVMHEALHNHYTQKSLSLSP |
| Fc monomer-5 | 128 | DKTHTCPPCPAPELLGGPSVFLFPPKPKDTLMISRTPEVTCVVVDVSHEDPE |
| | | VKFNWYVDGVEVHNAKTKPREEQYGSTYRVVSVLTVLHQDWLNGKEYK |
| | | CKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGN |
| | | VFSCSVMHEALHNHYTQKSLSLSPGK |
| Fc monomer-6 | 129 | DKTHTCPPCPAPELLGGPSVFLFPPKPKDTLMISRTPEVTCVVVDVSHEDPE |
| | | VKFNWYVDGVEVHNAKTKPREEQYGSTYRVVSVLTVLHQDWLNGKEYK |
| | | CKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGN |
| | | VFSCSVMHEALHNHYTQKSLSLSP |
| Fc monomer-7 | 130 | DKTHTCPPCPAPELLGGPSVFLFPPKPKDTLMISRTPEVTCVVVDVSHEDPE |
| | | VKFNWYVDGVEVHNAKTKPCEEQYNSTYRCVSVLTVLHQDWLNGKEYK |
| | | CKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGN |
| | | VFSCSVMHEALHNHYTQKSLSLSPGK |
| Fc monomer-8 | 131 | DKTHTCPPCPAPELLGGPSVFLFPPKPKDTLMISRTPEVTCVVVDVSHEDPE |
| | | VKFNWYVDGVEVHNAKTKPCEEQYNSTYRCVSVLTVLHQDWLNGKEYK |
| | | CKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGN |
| | | VFSCSVMHEALHNHYTQKSLSLSP |

Single-Chain Fc Domains

| | | |
|---|---|---|
| scFc-1 | 132 | DKTHTCPPCPAPELLGGPSVFLFPPKPKDTLMISRTPEVTCVVVDVSHEDPE |
| | | VKFNWYVDGVEVHNAKTKPCEEQYGSTYRCVSVLTVLHQDWLNGKEYK |
| | | CKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGN |
| | | VFSCSVMHEALHNHYTQKSLSLSPGKGGGGSGGGGSGGGGSGGGGSGGG |
| | | GSGGGGSDKTHTCPPCPAPELLGGPSVFLFPPKPKDTLMISRTPEVTCVVV |
| | | DVSHEDPEVKFNWYVDGVEVHNAKTKPCEEQYGSTYRCVSVLTVLHQD |
| | | WLNGKEYKCKVSNKALPAPIEKTISKAKGQPREPQVYTLPPSREEMTKNQ |
| | | VSLTCLVKGFYPSDIAVEWESNGQPENNYKTTPPVLDSDGSFFLYSKLTVD |
| | | KSRWQQGNVFSCSVMHEALHNHYTQKSLSLSPGK |
| scFc-2 | 133 | DKTHTCPPCPAPELLGGPSVFLFPPKPKDTLMISRTPEVTCVVVDVSHEDPE |
| | | VKFNWYVDGVEVHNAKTKPCEEQYGSTYRCVSVLTVLHQDWLNGKEYK |
| | | CKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGN |
| | | VFSCSVMHEALHNHYTQKSLSLSPGGGGSGGGGSGGGGSGGGGSGGGGS |
| | | GGGGSDKTHTCPPCPAPELLGGPSVFLFPPKPKDTLMISRTPEVTCVVVDV |
| | | SHEDPEVKFNWYVDGVEVHNAKTKPCEEQYGSTYRCVSVLTVLHQDWL |
| | | NGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSR |
| | | WQQGNVFSCSVMHEALHNHYTQKSLSLSP |
| scFc-3 | 134 | DKTHTCPPCPAPELLGGPSVFLFPPKPKDTLMISRTPEVTCVVVDVSHEDPE |
| | | VKFNWYVDGVEVHNAKTKPREEQYNSTYRVVSVLTVLHQDWLNGKEYK |
| | | CKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGN |
| | | VFSCSVMHEALHNHYTQKSLSLSPGKGGGGSGGGGSGGGGSGGGGSGGG |
| | | GSGGGGSDKTHTCPPCPAPELLGGPSVFLFPPKPKDTLMISRTPEVTCVVV |
| | | DVSHEDPEVKFNWYVDGVEVHNAKTKPREEQYNSTYRVVSVLTVLHQD |
| | | WLNGKEYKCKVSNKALPAPIEKTISKAKGQPREPQVYTLPPSREEMTKNQ |
| | | VSLTCLVKGFYPSDIAVEWESNGQPENNYKTTPPVLDSDGSFFLYSKLTVD |
| | | KSRWQQGNVFSCSVMHEALHNHYTQKSLSLSPGK |
| scFc-4 | 135 | DKTHTCPPCPAPELLGGPSVFLFPPKPKDTLMISRTPEVTCVVVDVSHEDPE |
| | | VKFNWYVDGVEVHNAKTKPREEQYNSTYRVVSVLTVLHQDWLNGKEYK |
| | | CKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGN |
| | | VFSCSVMHEALHNHYTQKSLSLSPGGGGSGGGGSGGGGSGGGGSGGGGS |
| | | GGGGSDKTHTCPPCPAPELLGGPSVFLFPPKPKDTLMISRTPEVTCVVVDV |
| | | SHEDPEVKFNWYVDGVEVHNAKTKPREEQYNSTYRVVSVLTVLHQDWL |
| | | NGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSR |
| | | WQQGNVFSCSVMHEALHNHYTQKSLSLSP |
| scFc-5 | 136 | DKTHTCPPCPAPELLGGPSVFLFPPKPKDTLMISRTPEVTCVVVDVSHEDPE |
| | | VKFNWYVDGVEVHNAKTKPREEQYGSTYRVVSVLTVLHQDWLNGKEYK |
| | | CKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGN |
| | | VFSCSVMHEALHNHYTQKSLSLSPGKGGGGSGGGGSGGGGSGGGGSGGG |
| | | GSGGGGSDKTHTCPPCPAPELLGGPSVFLFPPKPKDTLMISRTPEVTCVVV |
| | | DVSHEDPEVKFNWYVDGVEVHNAKTKPREEQYGSTYRVVSVLTVLHQD |
| | | WLNGKEYKCKVSNKALPAPIEKTISKAKGQPREPQVYTLPPSREEMTKNQ |
| | | VSLTCLVKGFYPSDIAVEWESNGQPENNYKTTPPVLDSDGSFFLYSKLTVD |
| | | KSRWQQGNVFSCSVMHEALHNHYTQKSLSLSPGK |
| scFc-6 | 137 | DKTHTCPPCPAPELLGGPSVFLFPPKPKDTLMISRTPEVTCVVVDVSHEDPE |
| | | VKFNWYVDGVEVHNAKTKPREEQYGSTYRVVSVLTVLHQDWLNGKEYK |
| | | CKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGN |
| | | VFSCSVMHEALHNHYTQKSLSLSPGGGGSGGGGSGGGGSGGGGSGGGGS |
| | | GGGGSDKTHTCPPCPAPELLGGPSVFLFPPKPKDTLMISRTPEVTCVVVDV |
| | | SHEDPEVKFNWYVDGVEVHNAKTKPREEQYGSTYRVVSVLTVLHQDWL |
| | | NGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSR |
| | | WQQGNVFSCSVMHEALHNHYTQKSLSLSP |
| scFc-7 | 138 | DKTHTCPPCPAPELLGGPSVFLFPPKPKDTLMISRTPEVTCVVVDVSHEDPE |
| | | VKFNWYVDGVEVHNAKTKPCEEQYNSTYRCVSVLTVLHQDWLNGKEYK |
| | | CKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGN |
| | | VFSCSVMHEALHNHYTQKSLSLSPGKGGGGSGGGGSGGGGSGGGGSGGG |
| | | GSGGGGSDKTHTCPPCPAPELLGGPSVFLFPPKPKDTLMISRTPEVTCVVV |
| | | DVSHEDPEVKFNWYVDGVEVHNAKTKPCEEQYNSTYRCVSVLTVLHQD |
| | | WLNGKEYKCKVSNKALPAPIEKTISKAKGQPREPQVYTLPPSREEMTKNQ |
| | | VSLTCLVKGFYPSDIAVEWESNGQPENNYKTTPPVLDSDGSFFLYSKLTVD |
| | | KSRWQQGNVFSCSVMHEALHNHYTQKSLSLSPGK |
| scFc-8 | 139 | DKTHTCPPCPAPELLGGPSVFLFPPKPKDTLMISRTPEVTCVVVDVSHEDPE |
| | | VKFNWYVDGVEVHNAKTKPCEEQYNSTYRCVSVLTVLHQDWLNGKEYK |
| | | CKVSNKALPAPIEKTISKAKGQPREPQVYTLPPSREEMTKNQVSLTCLVKG |
| | | FYPSDIAVEWESNGQPENNYKTTPPVLDSDGSFFLYSKLTVDKSRWQQGN |
| | | VFSCSVMHEALHNHYTQKSLSLSPGGGGSGGGGSGGGGSGGGGSGGGGS |
| | | GGGGSDKTHTCPPCPAPELLGGPSVFLFPPKPKDTLMISRTPEVTCVVVDV |
| | | SHEDPEVKFNWYVDGVEVHNAKTKPCEEQYNSTYRCVSVLTVLHQDWL |
| | | NGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSR |
| | | WQQGNVFSCSVMHEALHNHYTQKSLSLSP |

The Fc domain of the PSMA-targeted T-cell engaging molecules used in the methods of the invention can be any of the scFc domains set forth in Table 5 or a variant of these scFc domains. In one embodiment, the PSMA-targeted T-cell engaging molecules according to the invention comprise an Fc domain comprising an amino acid sequence that is at least 90% identical to a sequence selected from SEQ ID NOs: 132-139. In another embodiment, the PSMA-targeted T-cell engaging molecules according to the invention comprise an Fc domain comprising an amino acid sequence selected from SEQ ID NOs: 132-139. In a preferred embodiment, the PSMA-targeted T-cell engaging molecules according to the invention comprise an Fc domain comprising the amino acid sequence of SEQ ID NO: 132. In another preferred embodiment, the PSMA-targeted T-cell engaging molecules according to the invention comprise an Fc domain comprising the amino acid sequence of SEQ ID NO: 133.

In certain embodiments, the PSMA-targeted T-cell engaging molecules used in the methods of the invention comprise, in an amino to carboxyl order:
(i) a first domain that specifically binds to human PSMA comprising a first immunoglobulin heavy chain variable region (VH1) and a first immunoglobulin light chain variable region (VL1);
(ii) a second domain that specifically binds to human CD3 comprising a second immunoglobulin heavy chain variable region (VH2) and a second immunoglobulin light chain variable region (VL2); and
(iii) an Fc domain comprising two Fc monomers.
   In such embodiments, each Fc monomer may comprise an immunoglobulin hinge region, a CH2 domain, and a CH3 domain, wherein said two Fc monomers are fused to each other via a peptide linker, such as any of the peptide linkers described herein.

In some embodiments, the PSMA-targeted T-cell engaging molecules comprise, in amino to carboxyl order:
(i) a first domain that specifically binds to human PSMA comprising a VHI comprising a CDRH1 having the sequence of SEQ ID NO: 14 or SEQ ID NO. 15, a CDRH2 having a sequence selected from SEQ ID NOs: 16-19, and a CDRH3 having the sequence of SEQ ID NO: 20, and a VL1 comprising a CDRL1 having the sequence of SEQ ID NO: 5 or SEQ ID NO. 6, a CDRL2 having the sequence of SEQ ID NO: 7 or SEQ ID NO: 8, and a CDRL3 having a sequence selected from SEQ ID NOs: 9-13;
(ii) a second domain that specifically binds to human CD3 comprising a VH2 comprising a CDRH1 having a sequence selected from SEQ ID NOs: 48-53, a CDRH2 having a sequence selected from SEQ ID NOs: 54-58, and a CDRH3 having a sequence selected from SEQ ID NOs: 59-67, and a VL2 comprising a CDRL1 having a sequence selected from SEQ ID NOs: 41-43, a CDRL2 having the sequence of SEQ ID NO: 44 or SEQ ID NO. 45, and a CDRL3 having the sequence of SEQ ID NO: 46 or SEQ ID NO: 47; and
(iii) an Fc domain comprising two Fc monomers, each monomer comprising an immunoglobulin hinge region, a CH2 domain, and a CH3 domain, wherein said two Fc monomers are fused to each other via a peptide linker. In such embodiments, VHI comprises a sequence selected from SEQ ID NOs: 33-39 and VL1 comprises a sequence selected from SEQ ID NOs: 21-32. In these and other embodiments, VH2 comprises a sequence selected from SEQ ID NOs: 71-79 and VL2 comprises a sequence selected from SEQ ID NOs: 68-70. In one embodiment, VH1 comprises the sequence of SEQ ID NO: 33 and VL1 comprises the sequence of SEQ ID NO: 30. In a related embodiment, VH2 comprises the sequence of SEQ ID NO: 72 and VL2 comprises the sequence of SEQ ID NO: 70.

In a preferred embodiment, the PSMA-targeted T-cell engaging molecule comprises, in amino to carboxyl order:
(i) a first domain that specifically binds to human PSMA comprising a VHI comprising a CDRH1 having the sequence of SEQ ID NO: 14, a CDRH2 having the sequence of SEQ ID NO: 16, and a CDRH3 having the sequence of SEQ ID NO: 20, and a VL I comprising a CDRL1 having the sequence of SEQ ID NO: 5, a CDRL2 having the sequence of SEQ ID NO: 8, and a CDRL3 having the sequence of SEQ ID NO: 9;
(ii) a second domain that specifically binds to human CD3 comprising a VH2 comprising a CDRH1 having the sequence of SEQ ID NO: 49, a CDRH2 having the sequence of SEQ ID NO: 55, and a CDRH3 having the sequence of SEQ ID NO: 60, and a VL2 comprising a CDRL1 having the sequence of SEQ ID NO: 43, a CDRL2 having the sequence of SEQ ID NO: 44, and a CDRL3 having the sequence of SEQ ID NO: 47; and
(iii) an Fc domain comprising two Fc monomers, each monomer comprising an immunoglobulin hinge region, a CH2 domain, and a CH3 domain, wherein said two Fc monomers are fused to each other via a peptide linker.

In certain embodiments, peptide linkers, such as those described herein, connect the first domain to the second domain and/or the second domain to the Fc domain. Accordingly, in some embodiments, the PSMA-targeted T-cell engaging molecule according to the invention comprises, in amino to carboxyl order:
(i) a first domain that specifically binds to human PSMA;
(ii) a first peptide linker having an amino acid sequence selected from SEQ ID NOs: 81-83 and 91;
(iii) a second domain that specifically binds to human CD3;
(iv) a second peptide linker having an amino acid sequence selected from SEQ ID NOs: 80-83, and 89-91;
(v) a first Fc monomer;
(vi) a third peptide linker having an amino acid sequence selected from SEQ ID NOs: 85-88; and
(vii) a second Fc monomer.

In certain embodiments, the PSMA-targeted T-cell engaging molecule according to the invention comprises, in amino to carboxyl order:
(i) a first domain (e.g. anti-PSMA binding domain) having an amino acid sequence selected from SEQ ID NOs: 94-106;
(ii) a first peptide linker having an amino acid sequence selected from SEQ ID NOs: 81-83 and 91;
(iii) a second domain (e.g. anti-CD3 binding domain) having an amino acid sequence selected from SEQ ID NOs: 107-116;
(iv) a second peptide linker having an amino acid sequence selected from SEQ ID NOs: 80-83, and 89-91;
(v) a first Fc monomer having an amino acid sequence selected from SEQ ID NOs: 124-131;
(vi) a third peptide linker having an amino acid sequence selected from SEQ ID NOs: 85-88; and
(vii) a second Fc monomer having an amino acid sequence selected from SEQ ID NOs: 124-131.

In a preferred embodiment, the PSMA-targeted T-cell engaging molecule according to the invention comprises, in amino to carboxyl order:
(i) a first domain (e.g. anti-PSMA binding domain) having the amino acid sequence of SEQ ID NO: 104;
(ii) a first peptide linker having the amino acid sequence of SEQ ID NO: 81 or SEQ ID NO: 91;
(iii) a second domain (e.g. anti-CD3 binding domain) having the amino acid sequence of SEQ ID NO: 116;
(iv) a second peptide linker having the amino acid sequence of SEQ ID NO: 80 or SEQ ID NO: 81;
(v) a first Fc monomer having the amino acid sequence of SEQ ID NO: 124;
(vi) a third peptide linker having the amino acid sequence of SEQ ID NO: 86 or SEQ ID NO: 87; and
(vii) a second Fc monomer having the amino acid sequence of SEQ ID NO: 124.

In certain embodiments, the PSMA-targeted T-cell engaging molecules used in the methods of the invention are single chain polypeptides or single chain fusion proteins. As used herein, a “single chain polypeptide” or “single chain fusion protein” refers to a molecule consisting of only one polypeptide chain, i.e. all of the domains in the T-cell engaging molecule are linked together, optionally via peptide linkers, to form a single polypeptide chain. One example of such a single chain polypeptide or single chain fusion protein in the context of the present invention is a single chain polypeptide comprising, in an amino to carboxyl order, an anti-PSMA scFv domain, a first peptide linker, an anti-CD3 scFv domain, a second peptide linker, and an scFc domain. Exemplary PSMA-targeted T-cell engaging single chain polypeptides or single chain fusion proteins that can be used in the methods of the invention are set forth in Table 6 below. Other PSMA-targeted T-cell engaging single chain polypeptides or single chain fusion proteins suitable for use in the methods of the invention are described in WO 2017/134158, which is hereby incorporated by reference in its entirety.

TABLE 6
Exemplary PSMA-targeted T-cell Engaging Single Chain Polypeptides

| | SEQ ID | |
|---|---|---|
| =Designation | NO. | Amino Acid Sequence |
| scp-1 | 140 | QVQLVESGGGLVKPGESLRLSCAASGFTFSDYYMYWVRQAPGKCLEWVAIISDG |
| | | GYYTYYSDIIKGRFTISRDNAKNSLYLQMNSLKAEDTAVYYCARGFPLLRHGAM |
| | | DYWGQGTLVTVSSGGGGSGGGGSGGGGSDIQMTQSPSSLSASVGDRVTITCKAS |
| | | QNVDTNVAWYQQKPGQAPKSLIYSASYVYWDVPSRFSGSASGTDFTLTISSVQSE |
| | | DFATYYCQQYDQQLITFGCGTKLEIKSGGGGSEVQLVESGGGLVQPGGSLKLSCA |
| | | ASGFTFNKYAMNWVRQAPGKGLEWVARIRSKYNNYATYYADSVKDRFTISRDD |
| | | SKNTAYLQMNNLKTEDTAVYYCVRHGNFGNSYISYWAYWGQGTLVTVSSGGG |
| | | GSGGGGSGGGGSQTVVTQEPSLTVSPGGTVTLTCGSSTGAVTSGNYPNWVQQKP |
| | | GQAPRGLIGGTKFLAPGTPARFSGSLLGGKAALTLSGVQPEDEAEYYCVLWYSNR |
| | | WVFGGGTKLTVLGGGGDKTHTCPPCPAPELLGGPSVFLFPPKPKDTLMISRTPEV |
| | | TCVVVDVSHEDPEVKFNWYVDGVEVHNAKTKPCEEQYGSTYRCVSVLTVLHQD |
| | | WLNGKEYKCKVSNKALPAPIEKTISKAKGQPREPQVYTLPPSREEMTKNQVSLTC |
| | | LVKGFYPSDIAVEWESNGQPENNYKTTPPVLDSDGSFFLYSKLTVDKSRWQQGN |
| | | VFSCSVMHEALHNHYTQKSLSLSPGKGGGGSGGGGSGGGGSGGGGSGGGGSGG |
| | | GGSDKTHTCPPCPAPELLGGPSVFLFPPKPKDTLMISRTPEVTCVVVDVSHEDPEV |
| | | KFNWYVDGVEVHNAKTKPCEEQYGSTYRCVSVLTVLHQDWLNGKEYKCKVSN |
| | | KALPAPIEKTISKAKGQPREPQVYTLPPSREEMTKNQVSLTCLVKGFYPSDIAVEW |
| | | ESNGQPENNYKTTPPVLDSDGSFFLYSKLTVDKSRWQQGNVFSCSVMHEALHNH |
| | | YTQKSLSLSPGK |
| scp-2 | 141 | QVQLVESGGGLVKPGESLRLSCAASGFTFSDYYMYWVRQAPGKCLEWVAIISDG |
| | | GYYTYYSDIIKGRFTISRDNAKNSLYLQMNSLKAEDTAVYYCARGFPLLRHGAM |
| | | DYWGQGTLVTVSSGGGGSGGGGSGGGGSDIQMTQSPSSLSASVGDRVTITCKAS |
| | | QNVDTNVAWYQQKPGQAPKSLIYSASYVYWDVPSRFSGSASGTDFTLTISSVQSE |
| | | DFATYYCQQYDQQLITFGCGTKLEIKSGGGGSEVQLVESGGGLVQPGGSLKLSCA |
| | | ASGFTFNKYAMNWVRQAPGKGLEWVARIRSKYNNYATYYADSVKDRFTISRDD |
| | | SKNTAYLQMNNLKTEDTAVYYCVRHGNFGNSYISYWAYWGQGTLVTVSSGGG |
| | | GSGGGGSGGGGSQTVVTQEPSLTVSPGGTVTLTCGSSTGAVTSGNYPNWVQQKP |
| | | GQAPRGLIGGTKFLAPGTPARFSGSLLGGKAALTLSGVQPEDEAEYYCVLWYSNR |
| | | WVFGGGTKLTVLGGGGDKTHTCPPCPAPELLGGPSVFLFPPKPKDTLMISRTPEV |
| | | TCVVVDVSHEDPEVKFNWYVDGVEVHNAKTKPCEEQYGSTYRCVSVLTVLHQD |
| | | WLNGKEYKCKVSNKALPAPIEKTISKAKGQPREPQVYTLPPSREEMTKNQVSLTC |
| | | LVKGFYPSDIAVEWESNGQPENNYKTTPPVLDSDGSFFLYSKLTVDKSRWQQGN |
| | | VFSCSVMHEALHNHYTQKSLSLSPGGGGSGGGGSGGGGSGGGGSGGGGSGGGG |
| | | SDKTHTCPPCPAPELLGGPSVFLFPPKPKDTLMISRTPEVTCVVVDVSHEDPEVKF |
| | | NWYVDGVEVHNAKTKPCEEQYGSTYRCVSVLTVLHQDWLNGKEYKCKVSNKA |
| | | LPAPIEKTISKAKGQPREPQVYTLPPSREEMTKNQVSLTCLVKGFYPSDIAVEWES |
| | | NGQPENNYKTTPPVLDSDGSFFLYSKLTVDKSRWQQGNVFSCSVMHEALHNHYT |
| | | QKSLSLSP |
| scp-3 | 142 | QVQLVESGGGLVKPGESLRLSCAASGFTFSDYYMYWVRQAPGKCLEWVAIISDG |
| | | GYYTYYSDIIKGRFTISRDNAKNSLYLQMNSLKAEDTAVYYCARGFPLLRHGAM |
| | | DYWGQGTLVTVSSGGGGSGGGGSGGGGSDIQMTQSPSSLSASVGDRVTITCKAS |
| | | QNVDTNVAWYQQKPGQAPKSLIYSASYRYSDVPSRFSGSASGTDFTLTISSVQSE |
| | | DFATYYCQQYDQQLITFGCGTKLEIKSGGGGSEVQLVESGGGLVQPGGSLKLSCA |
| | | ASGFTFNKYAMNWVRQAPGKGLEWVARIRSKYNNYATYYADSVKDRFTISRDD |
| | | SKNTAYLQMNNLKTEDTAVYYCVRHGNFGNSYISYWAYWGQGTLVTVSSGGG |
| | | GSGGGGSGGGGSQTVVTQEPSLTVSPGGTVTLTCGSSTGAVTSGNYPNWVQQKP |
| | | GQAPRGLIGGTKFLAPGTPARFSGSLLGGKAALTLSGVQPEDEAEYYCVLWYSNR |
| | | WVFGGGTKLTVLGGGGDKTHTCPPCPAPELLGGPSVFLFPPKPKDTLMISRTPEV |
| | | TCVVVDVSHEDPEVKFNWYVDGVEVHNAKTKPCEEQYGSTYRCVSVLTVLHQD |
| | | WLNGKEYKCKVSNKALPAPIEKTISKAKGQPREPQVYTLPPSREEMTKNQVSLTC |
| | | LVKGFYPSDIAVEWESNGQPENNYKTTPPVLDSDGSFFLYSKLTVDKSRWQQGN |
| | | VFSCSVMHEALHNHYTQKSLSLSPGKGGGGSGGGGSGGGGSGGGGSGGGGSGG |
| | | GGSDKTHTCPPCPAPELLGGPSVFLFPPKPKDTLMISRTPEVTCVVVDVSHEDPEV |
| | | KFNWYVDGVEVHNAKTKPCEEQYGSTYRCVSVLTVLHQDWLNGKEYKCKVSN |
| | | KALPAPIEKTISKAKGQPREPQVYTLPPSREEMTKNQVSLTCLVKGFYPSDIAVEW |
| | | ESNGQPENNYKTTPPVLDSDGSFFLYSKLTVDKSRWQQGNVFSCSVMHEALHINH |
| | | YTQKSLSLSPGK |
| scp-4 | 143 | QVQLVESGGGLVKPGESLRLSCAASGFTFSDYYMYWVRQAPGKCLEWVAIISDG |
| | | GYYTYYSDIIKGRFTISRDNAKNSLYLQMNSLKAEDTAVYYCARGFPLLRHGAM |
| | | DYWGQGTLVTVSSGGGGSGGGGSGGGGSDIQMTQSPSSLSASVGDRVTITCKAS |
| | | QNVDTNVAWYQQKPGQAPKSLIYSASYRYSDVPSRFSGSASGTDFTLTISSVQSE |
| | | DFATYYCQQYDQVRLTFGCGTKLEIKSGGGGSEVQLVESGGGLVQPGGSLKLSC |
| | | AASGFTFNKYAMNWVRQAPGKGLEWVARIRSKYNNYATYYADSVKDRFTISRD |
| | | DSKNTAYLQMNNLKTEDTAVYYCVRHGNFGNSYISYWAYWGQGTLVTVSSGG |
| | | GGSGGGGSGGGGSQTVVTQEPSLTVSPGGTVTLTCGSSTGAVTSGNYPNWVQQK |
| | | PGQAPRGLIGGTKFLAPGTPARFSGSLLGGKAALTLSGVQPEDEAEYYCVLWYSN |
| | | RWVFGGGTKLTVLGGGGDKTHTCPPCPAPELLGGPSVFLFPPKPKDTLMISRTPE |
| | | VTCVVVDVSHEDPEVKFNWYVDGVEVHNAKTKPCEEQYGSTYRCVSVLTVLHQ |
| | | DWLNGKEYKCKVSNKALPAPIEKTISKAKGQPREPQVYTLPPSREEMTKNQVSLT |
| | | CLVKGFYPSDIAVEWESNGQPENNYKTTPPVLDSDGSFFLYSKLTVDKSRWQQG |
| | | NVFSCSVMHEALHNHYTQKSLSLSPGKGGGGSGGGGSGGGGSGGGGSGGGGSG |
| | | GGGSDKTHTCPPCPAPELLGGPSVFLFPPKPKDTLMISRTPEVTCVVVDVSHEDPE |
| | | VKFNWYVDGVEVHNAKTKPCEEQYGSTYRCVSVLTVLHQDWLNGKEYKCKVS |
| | | NKALPAPIEKTISKAKGQPREPQVYTLPPSREEMTKNQVSLTCLVKGFYPSDIAVE |
| | | WESNGQPENNYKTTPPVLDSDGSFFLYSKLTVDKSRWQQGNVFSCSVMHEALHN |
| | | HYTQKSLSLSPGK |
| scp-5 | 144 | QVQLVESGGGLVKPGESLRLSCAASGFTFSDYYMYWVRQAPGKCLEWVAIISDH |
| | | HHWTYYSDIIKGRFTISRDNAKNSLYLQMNSLKAEDTAVYYCARGFPLLRHGAM |
| | | DYWGQGTLVTVSSGGGGGGGGSGGGGSDIQMTQSPSSLSASVGDRVTITCKAS |
| | | QNVDTNVAWYQQKPGQAPKSLIYSASYRYSDVPSRFSGSASGTDFTLTISSVQSE |
| | | DFATYYCQQYDSYPYTFGCGTKLEIKSGGGGSEVQLVESGGGLVQPGGSLKLSC |
| | | AASGFTFNKYAMNWVRQAPGKGLEWVARIRSKYNNYATYYADSVKDRFTISRD |
| | | DSKNTAYLQMNNLKTEDTAVYYCVRHGNFGNSYISYWAYWGQGTLVTVSSGG |
| | | GGSGGGGSGGGGSQTVVTQEPSLTVSPGGTVTLTCGSSTGAVTSGNYPNWVQQK |
| | | PGQAPRGLIGGTKFLAPGTPARFSGSLLGGKAALTLSGVQPEDEAEYYCVLWYSN |
| | | RWVFGGGTKLTVLGGGGDKTHTCPPCPAPELLGGPSVFLFPPKPKDTLMISRTPE |
| | | VTCVVVDVSHEDPEVKFNWYVDGVEVHNAKTKPCEEQYGSTYRCVSVLTVLHQ |
| | | DWLNGKEYKCKVSNKALPAPIEKTISKAKGQPREPQVYTLPPSREEMTKNQVSLT |
| | | CLVKGFYPSDIAVEWESNGQPENNYKTTPPVLDSDGSFFLYSKLTVDKSRWQQG |
| | | NVFSCSVMHEALHNHYTQKSLSLSPGKGGGGGGGGSGGGGSGGGGSGGGGSG |
| | | GGGSDKTHTCPPCPAPELLGGPSVFLFPPKPKDTLMISRTPEVTCVVVDVSHEDPE |
| | | VKFNWYVDGVEVHNAKTKPCEEQYGSTYRCVSVLTVLHQDWLNGKEYKCKVS |
| | | NKALPAPIEKTISKAKGQPREPQVYTLPPSREEMTKNQVSLTCLVKGFYPSDIAVE |
| | | WESNGQPENNYKTTPPVLDSDGSFFLYSKLTVDKSRWQQGNVFSCSVMHEALHN |
| | | HYTQKSLSLSPGK |
| scp-6 | 145 | QVQLVESGGGLVKPGESLRLSCAASGFTFSDYYMYWVRQAPGKCLEWVAIISDG |
| | | GYYTYYSDIIKGRFTISRDNAKNSLYLQMNSLKAEDTAVYYCARGFPLLRHGAM |
| | | DYWGQGTLVTVSSGGGGSGGGGSGGGGSDIQMTQSPSSLSASVGDRVTITCKAS |
| | | QNVDTNVAWYQQKPGQAPKSLIYSASYRYSDVPSRFSGSASGTDFTLTISSVQSE |
| | | DFATYYCQQYDRQPLTFGCGTKLEIKSGGGGSEVQLVESGGGLVQPGGSLKLSC |
| | | AASGFTFNKYAMNWVRQAPGKGLEWVARIRSKYNNYATYYADSVKDRFTISRD |
| | | DSKNTAYLQMNNLKTEDTAVYYCVRHGNFGNSYISYWAYWGQGTLVTVSSGG |
| | | GGSGGGGGGGGSQTVVTQEPSLTVSPGGTVTLTCGSSTGAVTSGNYPNWVQQK |
| | | PGQAPRGLIGGTKFLAPGTPARFSGSLLGGKAALTLSGVQPEDEAEYYCVLWYSN |
| | | RWVFGGGTKLTVLGGGGDKTHTCPPCPAPELLGGPSVFLFPPKPKDTLMISRTPE |
| | | VTCVVVDVSHEDPEVKFNWYVDGVEVHNAKTKPCEEQYGSTYRCVSVLTVLHQ |
| | | DWLNGKEYKCKVSNKALPAPIEKTISKAKGQPREPQVYTLPPSREEMTKNQVSLT |
| | | CLVKGFYPSDIAVEWESNGQPENNYKTTPPVLDSDGSFFLYSKLTVDKSRWQQG |
| | | NVFSCSVMHEALHNHYTQKSLSLSPGKGGGGSGGGGSGGGGSGGGGSGGGGSG |
| | | GGGSDKTHTCPPCPAPELLGGPSVFLFPPKPKDTLMISRTPEVTCVVVDVSHEDPE |
| | | VKFNWYVDGVEVHNAKTKPCEEQYGSTYRCVSVLTVLHQDWLNGKEYKCKVS |
| | | NKALPAPIEKTISKAKGQPREPQVYTLPPSREEMTKNQVSLTCLVKGFYPSDIAVE |
| | | WESNGQPENNYKTTPPVLDSDGSFFLYSKLTVDKSRWQQGNVFSCSVMHEALHN |
| | | HYTQKSLSLSPGK |
| scp-7 | 146 | QVQLVESGGGLVKPGESLRLSCAASGFTFSDYYMYWVRQAPGKCLEWVAIISDG |
| | | GYYTYYSDIIKGRFTISRDNAKNSLYLQMNSLKAEDTAVYYCARGFPLLRHGAM |
| | | DYWGQGTLVTVSSGGGGSGGGGSGGGGSDIQMTQSPSSLSASVGDRVTITCKAS |
| | | QNVDTNVAWYQQKPGQAPKSLIYSASYRYSDVPSRFSGSASGTDFTLTISSVQSE |
| | | DFATYYCQQYDAPMGTFGCGTKLEIKSGGGGSEVQLVESGGGLVQPGGSLKLSC |
| | | AASGFTFNKYAMNWVRQAPGKGLEWVARIRSKYNNYATYYADSVKDRFTISRD |
| | | DSKNTAYLQMNNLKTEDTAVYYCVRHGNFGNSYISYWAYWGQGTLVTVSSGG |
| | | GGSGGGGSGGGGSQTVVTQEPSLTVSPGGTVTLTCGSSTGAVTSGNYPNWVQQK |
| | | PGQAPRGLIGGTKFLAPGTPARFSGSLLGGKAALTLSGVQPEDEAEYYCVLWYSN |
| | | RWVFGGGTKLTVLGGGGDKTHTCPPCPAPELLGGPSVFLFPPKPKDTLMISRTPE |
| | | VTCVVVDVSHEDPEVKFNWYVDGVEVHNAKTKPCEEQYGSTYRCVSVLTVLHQ |
| | | DWLNGKEYKCKVSNKALPAPIEKTISKAKGQPREPQVYTLPPSREEMTKNQVSLT |
| | | CLVKGFYPSDIAVEWESNGQPENNYKTTPPVLDSDGSFFLYSKLTVDKSRWQQG |
| | | NVFSCSVMHEALHNHYTQKSLSLSPGKGGGGSGGGGGGGGSGGGGSGGGGSG |
| | | GGGSDKTHTCPPCPAPELLGGPSVFLFPPKPKDTLMISRTPEVTCVVVDVSHEDPE |
| | | VKFNWYVDGVEVHNAKTKPCEEQYGSTYRCVSVLTVLHQDWLNGKEYKCKVS |
| | | NKALPAPIEKTISKAKGQPREPQVYTLPPSREEMTKNQVSLTCLVKGFYPSDIAVE |
| | | WESNGQPENNYKTTPPVLDSDGSFFLYSKLTVDKSRWQQGNVFSCSVMHEALHN |
| | | HYTQKSLSLSPGK |
| scp-8 | 147 | QVQLVESGGGLVKPGESLRLSCAASGFTFSDYYMNWVRQAPGKGLEWVAIISDN |
| | | KHQTYYSDIIKGRFAISRDNAKNSLYLQMNSLKAEDTAVYYCARGFPLLRHGAM |
| | | DYWGQGTLVTVSSGGGGSGGGGSGGGGSDIQMTQSPSSLSASVGDRVTITCKAS |
| | | QNVDTNVAWYQQKPGQAPKSLIYSASYRYSDVPSRFSGSASGTDFTLTISSLQSED |
| | | FATYYCQQYDSYPYTFGGGTKLEIKSGGGGSEVQLVESGGGLVQPGGSLKLSCA |
| | | ASGFTFNKYAMNWVRQAPGKGLEWVARIRSKYNNYATYYADSVKDRFTISRDD |
| | | SKNTAYLQMNNLKTEDTAVYYCVRHGNFGNSYISYWAYWGQGTLVTVSSGGG |
| | | GSGGGGSGGGGSQTVVTQEPSLTVSPGGTVTLTCGSSTGAVTSGNYPNWVQQKP |
| | | GQAPRGLIGGTKFLAPGTPARFSGSLLGGKAALTLSGVQPEDEAEYYCVLWYSNR |
| | | WVFGGGTKLTVLGGGGDKTHTCPPCPAPELLGGPSVFLFPPKPKDTLMISRTPEV |
| | | TCVVVDVSHEDPEVKFNWYVDGVEVHNAKTKPCEEQYGSTYRCVSVLTVLHQD |
| | | WLNGKEYKCKVSNKALPAPIEKTISKAKGQPREPQVYTLPPSREEMTKNQVSLTC |
| | | LVKGFYPSDIAVEWESNGQPENNYKTTPPVLDSDGSFFLYSKLTVDKSRWQQGN |
| | | VFSCSVMHEALHNHYTQKSLSLSPGKGGGGSGGGGSGGGGSGGGGSGGGGSGG |
| | | GGSDKTHTCPPCPAPELLGGPSVFLFPPKPKDTLMISRTPEVTCVVVDVSHEDPEV |
| | | KFNWYVDGVEVHNAKTKPCEEQYGSTYRCVSVLTVLHQDWLNGKEYKCKVSN |
| | | KALPAPIEKTISKAKGQPREPQVYTLPPSREEMTKNQVSLTCLVKGFYPSDIAVEW |
| | | ESNGQPENNYKTTPPVLDSDGSFFLYSKLTVDKSRWQQGNVFSCSVMHEALHINH |
| | | YTQKSLSLSPGK |
| scp-9 | 148 | QVQLVESGGGLVKPGESLRLSCAASGFTFSDYYMNWVRQAPGKCLEWVAIISDN |
| | | KHQTYYSDIIKGRFAISRDNAKNSLYLQMNSLKAEDTAVYYCARGFPLLRHGAM |
| | | DYWGQGTLVTVSSGGGGSGGGGSGGGGSDIQMTQSPSSLSASVGDRVTITCKAS |
| | | QNVDTNVAWYQQKPGQAPKSLIYSASYRYSDVPSRFSGSASGTDFTLTISSLQSED |
| | | FATYYCQQYDSYPYTFGCGTKLEIKSGGGGSEVQLVESGGGLVQPGGSLKLSCA |
| | | ASGFTFNKYAMNWVRQAPGKGLEWVARIRSKYNNYATYYADSVKDRFTISRDD |
| | | SKNTAYLQMNNLKTEDTAVYYCVRHGNFGNSYISYWAYWGQGTLVTVSSGGG |
| | | GSGGGGSGGGGSQTVVTQEPSLTVSPGGTVTLTCGSSTGAVTSGNYPNWVQQKP |
| | | GQAPRGLIGGTKFLAPGTPARFSGSLLGGKAALTLSGVQPEDEAEYYCVLWYSNR |
| | | WVFGGGTKLTVLGGGGDKTHTCPPCPAPELLGGPSVFLFPPKPKDTLMISRTPEV |
| | | TCVVVDVSHEDPEVKFNWYVDGVEVHNAKTKPCEEQYGSTYRCVSVLTVLHQD |
| | | WLNGKEYKCKVSNKALPAPIEKTISKAKGQPREPQVYTLPPSREEMTKNQVSLTC |
| | | LVKGFYPSDIAVEWESNGQPENNYKTTPPVLDSDGSFFLYSKLTVDKSRWQQGN |
| | | VFSCSVMHEALHNHYTQKSLSLSPGKGGGGSGGGGSGGGGGGGGSGGGGSGG |
| | | GGSDKTHTCPPCPAPELLGGPSVFLFPPKPKDTLMISRTPEVTCVVVDVSHEDPEV |
| | | KFNWYVDGVEVHNAKTKPCEEQYGSTYRCVSVLTVLHQDWLNGKEYKCKVSN |
| | | KALPAPIEKTISKAKGQPREPQVYTLPPSREEMTKNQVSLTCLVKGFYPSDIAVEW |
| | | ESNGQPENNYKTTPPVLDSDGSFFLYSKLTVDKSRWQQGNVFSCSVMHEALHNH |
| | | YTQKSLSLSPGK |
| scp-10 | 149 | QVQLVESGGGLVKPGESLRLSCAASGFTFSDYYMYWVRQAPGKGLEWVAIISDG |
| | | GYYTYYSDIIKGRFTISRDNAKNSLYLQMNSLKAEDTAVYYCARGFPLLRHGAM |
| | | DYWGQGTLVTVSSGGGGSGGGGSGGGGSDIQMTQSPSSLSASVGDRVTITCKAS |
| | | QNVDTNVAWYQQKPGQAPKSLIYSASYRYSDVPSRFSGSASGTDFTLTISSVQSE |
| | | DFATYYCQQYDSYPYTFGGGTKLEIKSGGGGSEVQLVESGGGLVQPGGSLKLSC |
| | | AASGFTFNKYAMNWVRQAPGKGLEWVARIRSKYNNYATYYADSVKDRFTISRD |
| | | DSKNTAYLQMNNLKTEDTAVYYCVRHGNFGNSYISYWAYWGQGTLVTVSSGG |
| | | GGSGGGGSGGGGSQTVVTQEPSLTVSPGGTVTLTCGSSTGAVTSGNYPNWVQQK |
| | | PGQAPRGLIGGTKFLAPGTPARFSGSLLGGKAALTLSGVQPEDEAEYYCVLWYSN |
| | | RWVFGGGTKLTVLGGGGDKTHTCPPCPAPELLGGPSVFLFPPKPKDTLMISRTPE |
| | | VTCVVVDVSHEDPEVKFNWYVDGVEVHNAKTKPCEEQYGSTYRCVSVLTVLHQ |
| | | DWLNGKEYKCKVSNKALPAPIEKTISKAKGQPREPQVYTLPPSREEMTKNQVSLT |
| | | CLVKGFYPSDIAVEWESNGQPENNYKTTPPVLDSDGSFFLYSKLTVDKSRWQQG |
| | | NVFSCSVMHEALHNHYTQKSLSLSPGKGGGGSGGGGSGGGGSGGGGSGGGGSG |
| | | GGGSDKTHTCPPCPAPELLGGPSVFLFPPKPKDTLMISRTPEVTCVVVDVSHEDPE |
| | | VKFNWYVDGVEVHNAKTKPCEEQYGSTYRCVSVLTVLHQDWLNGKEYKCKVS |
| | | NKALPAPIEKTISKAKGQPREPQVYTLPPSREEMTKNQVSLTCLVKGFYPSDIAVE |
| | | WESNGQPENNYKTTPPVLDSDGSFFLYSKLTVDKSRWQQGNVFSCSVMHEALHN |
| | | HYTQKSLSLSPGK |
| scp-11 | 150 | QVQLVESGGGLVKPGESLRLSCAASGFTFSDYYMYWVRQAPGKCLEWVAIISDG |
| | | GYYTYYSDIIKGRFTISRDNAKNSLYLQMNSLKAEDTAVYYCARGFPLLRHGAM |
| | | DYWGQGTLVTVSSGGGGSGGGGSGGGGSDIQMTQSPSSLSASVGDRVTITCKAS |
| | | QNVDTNVAWYQQKPGQAPKSLIYSASYRYSDVPSRFSGSASGTDFTLTISSVQSE |
| | | DFATYYCQQYDSYPYTFGCGTKLEIKSGGGGSEVQLVESGGGLVQPGGSLKLSC |
| | | AASGFTFNKYAMNWVRQAPGKGLEWVARIRSKYNNYATYYADSVKDRFTISRD |
| | | DSKNTAYLQMNNLKTEDTAVYYCVRHGNFGNSYISYWAYWGQGTLVTVSSGG |
| | | GGSGGGGSGGGGSQTVVTQEPSLTVSPGGTVTLTCGSSTGAVTSGNYPNWVQQK |
| | | PGQAPRGLIGGTKFLAPGTPARFSGSLLGGKAALTLSGVQPEDEAEYYCVLWYSN |
| | | RWVFGGGTKLTVLGGGGDKTHTCPPCPAPELLGGPSVFLFPPKPKDTLMISRTPE |
| | | VTCVVVDVSHEDPEVKFNWYVDGVEVHNAKTKPCEEQYGSTYRCVSVLTVLHQ |
| | | DWLNGKEYKCKVSNKALPAPIEKTISKAKGQPREPQVYTLPPSREEMTKNQVSLT |
| | | CLVKGFYPSDIAVEWESNGQPENNYKTTPPVLDSDGSFFLYSKLTVDKSRWQQG |
| | | NVFSCSVMHEALHNHYTQKSLSLSPGKGGGGSGGGGSGGGGSGGGGSGGGGSG |
| | | GGGSDKTHTCPPCPAPELLGGPSVFLFPPKPKDTLMISRTPEVTCVVVDVSHEDPE |
| | | VKFNWYVDGVEVHNAKTKPCEEQYGSTYRCVSVLTVLHQDWLNGKEYKCKVS |
| | | NKALPAPIEKTISKAKGQPREPQVYTLPPSREEMTKNQVSLTCLVKGFYPSDIAVE |
| | | WESNGQPENNYKTTPPVLDSDGSFFLYSKLTVDKSRWQQGNVFSCSVMHEALHN |
| | | HYTQKSLSLSPGK |
| scp-12 | 151 | QVQLVESGGGLVKPGESLRLSCAASGFTFSDYYMYWVRQAPGKGLEWVAIISDG |
| | | GYYTYYSDIIKGRFTISRDNAKNSLYLQMNSLKAEDTAVYYCARGFPLLRHGAM |
| | | DYWGQGTLVTVSSGGGGSGGGGSGGGGSDIQMTQSPSSLSASVGDRVTITCKAS |
| | | QNVDTNVAWYQQKPGQAPKSLIYSASYVYWDVPSRFSGSASGTDFTLTISSVQSE |
| | | DFATYYCQQYDQQLITFGGGTKLEIKSGGGGSEVQLVESGGGLVQPGGSLKLSCA |
| | | ASGFTENKYAMNWVRQAPGKGLEWVARIRSKYNNYATYYADSVKDRFTISRDD |
| | | SKNTAYLQMNNLKTEDTAVYYCVRHGNFGNSYISYWAYWGQGTLVTVSSGGG |
| | | GSGGGGSGGGGSQTVVTQEPSLTVSPGGTVTLTCGSSTGAVTSGNYPNWVQQKP |
| | | GQAPRGLIGGTKFLAPGTPARFSGSLLGGKAALTLSGVQPEDEAEYYCVLWYSNR |
| | | WVFGGGTKLTVLGGGGDKTHTCPPCPAPELLGGPSVFLFPPKPKDTLMISRTPEV |
| | | TCVVVDVSHEDPEVKFNWYVDGVEVHNAKTKPCEEQYGSTYRCVSVLTVLHQD |
| | | WLNGKEYKCKVSNKALPAPIEKTISKAKGQPREPQVYTLPPSREEMTKNQVSLTC |
| | | LVKGFYPSDIAVEWESNGQPENNYKTTPPVLDSDGSFFLYSKLTVDKSRWQQGN |
| | | VFSCSVMHEALHNHYTQKSLSLSPGKGGGGSGGGGSGGGGSGGGGSGGGGSGG |
| | | GGSDKTHTCPPCPAPELLGGPSVFLFPPKPKDTLMISRTPEVTCVVVDVSHEDPEV |
| | | KFNWYVDGVEVHNAKTKPCEEQYGSTYRCVSVLTVLHQDWLNGKEYKCKVSN |
| | | KALPAPIEKTISKAKGQPREPQVYTLPPSREEMTKNQVSLTCLVKGFYPSDIAVEW |
| | | ESNGQPENNYKTTPPVLDSDGSFFLYSKLTVDKSRWQQGNVFSCSVMHEALHINH |
| | | YTQKSLSLSPGK |
| scp-13 | 152 | QVQLVESGGGLVKPGESLRLSCAASGFTFSDYYMYWVRQAPGKGLEWVAIISDA |
| | | GYYTYYSDIIKGRFTISRDNAKNSLYLQMNSLKAEDTAVYYCARGFPLLRHGAM |
| | | DYWGQGTLVTVSSGGGGSGGGGSGGGGSDIQMTQSPSSLSASVGDRVTITCKAS |
| | | QNVDANVAWYQQKPGQAPKSLIYSASYVYWDVPSRFSGSASGTDFTLTISSVQSE |
| | | DFATYYCQQYDQQLITFGGGTKLEIKSGGGGSEVQLVESGGGLVQPGGSLKLSCA |
| | | ASGFTFNKYAMNWVRQAPGKGLEWVARIRSKYNNYATYYADSVKDRFTISRDD |
| | | SKNTAYLQMNNLKTEDTAVYYCVRHGNFGNSYISYWAYWGQGTLVTVSSGGG |
| | | GSGGGGSGGGGSQTVVTQEPSLTVSPGGTVTLTCGSSTGAVTSGNYPNWVQQKP |
| | | GQAPRGLIGGTKFLAPGTPARFSGSLLGGKAALTLSGVQPEDEAEYYCVLWYSNR |
| | | WVFGGGTKLTVLGGGGDKTHTCPPCPAPELLGGPSVFLFPPKPKDTLMISRTPEV |
| | | TCVVVDVSHEDPEVKFNWYVDGVEVHNAKTKPCEEQYGSTYRCVSVLTVLHQD |
| | | WLNGKEYKCKVSNKALPAPIEKTISKAKGQPREPQVYTLPPSREEMTKNQVSLTC |
| | | LVKGFYPSDIAVEWESNGQPENNYKTTPPVLDSDGSFFLYSKLTVDKSRWQQGN |
| | | VFSCSVMHEALHNHYTQKSLSLSPGKGGGGSGGGGSGGGGSGGGGSGGGGSGG |
| | | GGSDKTHTCPPCPAPELLGGPSVFLFPPKPKDTLMISRTPEVTCVVVDVSHEDPEV |
| | | KFNWYVDGVEVHNAKTKPCEEQYGSTYRCVSVLTVLHQDWLNGKEYKCKVSN |
| | | KALPAPIEKTISKAKGQPREPQVYTLPPSREEMTKNQVSLTCLVKGFYPSDIAVEW |
| | | ESNGQPENNYKTTPPVLDSDGSFFLYSKLTVDKSRWQQGNVFSCSVMHEALHNH |
| | | YTQKSLSLSPGK |
| scp-14 | 153 | QVQLVESGGGLVKPGESLRLSCAASGFTFSDYYMYWVRQAPGKCLEWVAIISDA |
| | | GYYTYYSDIIKGRFTISRDNAKNSLYLQMNSLKAEDTAVYYCARGFPLLRHGAM |
| | | DYWGQGTLVTVSSGGGGSGGGGSGGGGSDIQMTQSPSSLSASVGDRVTITCKAS |
| | | QNVDANVAWYQQKPGQAPKSLIYSASYVYWDVPSRFSGSASGTDFTLTISSVQSE |
| | | DFATYYCQQYDQQLITFGCGTKLEIKSGGGGSEVQLVESGGGLVQPGGSLKLSCA |
| | | ASGFTFNKYAMNWVRQAPGKGLEWVARIRSKYNNYATYYADSVKDRFTISRDD |
| | | SKNTAYLQMNNLKTEDTAVYYCVRHGNFGNSYISYWAYWGQGTLVTVSSGGG |
| | | GSGGGGSGGGGSQTVVTQEPSLTVSPGGTVILTCGSSTGAVTSGNYPNWVQQKP |
| | | GQAPRGLIGGTKFLAPGTPARFSGSLLGGKAALTLSGVQPEDEAEYYCVLWYSNR |
| | | WVFGGGTKLTVLGGGGDKTHTCPPCPAPELLGGPSVFLFPPKPKDTLMISRTPEV |
| | | TCVVVDVSHEDPEVKFNWYVDGVEVHNAKTKPCEEQYGSTYRCVSVLTVLHQD |
| | | WLNGKEYKCKVSNKALPAPIEKTISKAKGQPREPQVYTLPPSREEMTKNQVSLTC |
| | | LVKGFYPSDIAVEWESNGQPENNYKTTPPVLDSDGSFFLYSKLTVDKSRWQQGN |
| | | VFSCSVMHEALHNHYTQKSLSLSPGKGGGGSGGGGSGGGGSGGGGSGGGGSGG |
| | | GGSDKTHTCPPCPAPELLGGPSVFLFPPKPKDTLMISRTPEVTCVVVDVSHEDPEV |
| | | KFNWYVDGVEVHNAKTKPCEEQYGSTYRCVSVLTVLHQDWLNGKEYKCKVSN |
| | | KALPAPIEKTISKAKGQPREPQVYTLPPSREEMTKNQVSLTCLVKGFYPSDIAVEW |
| | | ESNGQPENNYKTTPPVLDSDGSFFLYSKLTVDKSRWQQGNVFSCSVMHEALHNH |
| | | YTQKSLSLSPGK |
| scp-15 | 154 | QVQLVESGGGLVKPGESLRLSCAASGFTFSDYYMYWVRQAPGKCLEWVAIISDG |
| | | GYYTYYSDIIKGRFTISRDNAKNSLYLQMNSLKAEDTAVYYCARGFPLLRHGAM |
| | | DYWGQGTLVTVSSGGGGSGGGGSGGGGSDIQMTQSPSSLSASVGDRVTITCKAS |
| | | QNVDTNVAWYQQKPGQAPKSLIYSASYVYWDVPSRFSGSASGTDFTLTISSVQSE |
| | | DFATYYCQQYDQQLITFGCGTKLEIKSGGGGSEVQLVESGGGLVQPGGSLKLSCA |
| | | ASGFTFNKYAMNWVRQAPGKGLEWVARIRSKYNNYATYYADSVKDRFTISRDD |
| | | SKNTAYLQMNNLKTEDTAVYYCVRHGNFGNSYISYWAYWGQGTLVTVSSGGG |
| | | GSGGGGSGGGGSQTVVTQEPSLTVSPGGTVTLTCGSSTGAVTSGYYPNWVQQKP |
| | | GQAPRGLIGGTKFLAPGTPARFSGSLLGGKAALTLSGVQPEDEAEYYCALWYSNR |
| | | WVFGGGTKLTVLGGGGDKTHTCPPCPAPELLGGPSVFLFPPKPKDTLMISRTPEV |
| | | TCVVVDVSHEDPEVKFNWYVDGVEVHNAKTKPCEEQYGSTYRCVSVLTVLHQD |
| | | WLNGKEYKCKVSNKALPAPIEKTISKAKGQPREPQVYTLPPSREEMTKNQVSLTC |
| | | LVKGFYPSDIAVEWESNGQPENNYKTTPPVLDSDGSFFLYSKLTVDKSRWQQGN |
| | | VFSCSVMHEALHNHYTQKSLSLSPGKGGGGSGGGGSGGGGSGGGGSGGGGSGG |
| | | GGSDKTHTCPPCPAPELLGGPSVFLFPPKPKDTLMISRTPEVTCVVVDVSHEDPEV |
| | | KFNWYVDGVEVHNAKTKPCEEQYGSTYRCVSVLTVLHQDWLNGKEYKCKVSN |
| | | KALPAPIEKTISKAKGQPREPQVYTLPPSREEMTKNQVSLTCLVKGFYPSDIAVEW |
| | | ESNGQPENNYKTTPPVLDSDGSFFLYSKLTVDKSRWQQGNVFSCSVMHEALHNH |
| | | YTQKSLSLSPGK |
| scp-16 | 155 | QVQLVESGGGLVKPGESLRLSCAASGFTFSDYYMYWVRQAPGKCLEWVAIISDG |
| | | GYYTYYSDIIKGRFTISRDNAKNSLYLQMNSLKAEDTAVYYCARGFPLLRHGAM |
| | | DYWGQGTLVTVSSGGGGSGGGGSGGGGSDIQMTQSPSSLSASVGDRVTITCKAS |
| | | QNVDTNVAWYQQKPGQAPKSLIYSASYVYWDVPSRFSGSASGTDFTLTISSVQSE |
| | | DFATYYCQQYDQQLITFGCGTKLEIKSGGGGSEVQLVESGGGLVQPGGSLKLSCA |
| | | ASGFTFNSYAMNWVRQAPGKGLEWVARIRSKYNNYATYYADSVKGRFTISRDD |
| | | SKNTAYLQMNNLKTEDTAVYYCVRHGNFGNSYVSWWAYWGQGTLVTVSSGG |
| | | GGSGGGGSGGGGSQTVVTQEPSLTVSPGGTVTLTCGSSTGAVTSGNYPNWVQQK |
| | | PGQAPRGLIGGTKFLAPGTPARFSGSLLGGKAALTLSGVQPEDEAEYYCVLWYSN |
| | | RWVFGGGTKLTVLGGGGDKTHTCPPCPAPELLGGPSVFLFPPKPKDTLMISRTPE |
| | | VTCVVVDVSHEDPEVKFNWYVDGVEVHNAKTKPCEEQYGSTYRCVSVLTVLHQ |
| | | DWLNGKEYKCKVSNKALPAPIEKTISKAKGQPREPQVYTLPPSREEMTKNQVSLT |
| | | CLVKGFYPSDIAVEWESNGQPENNYKTTPPVLDSDGSFFLYSKLTVDKSRWQQG |
| | | NVFSCSVMHEALHNHYTQKSLSLSPGKGGGGSGGGGSGGGGSGGGGSGGGGSG |
| | | GGGSDKTHTCPPCPAPELLGGPSVFLFPPKPKDTLMISRTPEVTCVVVDVSHEDPE |
| | | VKFNWYVDGVEVHNAKTKPCEEQYGSTYRCVSVLTVLHQDWLNGKEYKCKVS |
| | | NKALPAPIEKTISKAKGQPREPQVYTLPPSREEMTKNQVSLTCLVKGFYPSDIAVE |
| | | WESNGQPENNYKTTPPVLDSDGSFFLYSKLTVDKSRWQQGNVFSCSVMHEALHN |
| | | HYTQKSLSLSPGK |
| scp-17 | 156 | QVQLVESGGGLVKPGESLRLSCAASGFTFSDYYMYWVRQAPGKCLEWVAIISDG |
| | | GYYTYYSDIIKGRFTISRDNAKNSLYLQMNSLKAEDTAVYYCARGFPLLRHGAM |
| | | DYWGQGTLVTVSSGGGGSGGGGSGGGGSDIQMTQSPSSLSASVGDRVTITCKAS |
| | | QNVDTNVAWYQQKPGQAPKSLIYSASYRYSDVPSRFSGSASGTDFTLTISSVQSE |
| | | DFATYYCQQYDSYPYTFGCGTKLEIKSGGGGSEVQLVESGGGLVQPGGSLKLSC |
| | | AASGFTFNKYAMNWVRQAPGKGLEWVARIRSKYNNYATYYADSVKDRFTISRD |
| | | DSKNTAYLQMNNLKTEDTAVYYCVRHGNFGNSYISYWAYWGQGTLVTVSSGG |
| | | GGSGGGGSGGGGSQTVVTQEPSLTVSPGGTVTLTCGSSTGAVTSGYYPNWVQQK |
| | | PGQAPRGLIGGTKFLAPGTPARFSGSLLGGKAALTLSGVQPEDEAEYYCALWYSN |
| | | RWVFGGGTKLTVLGGGGDKTHTCPPCPAPELLGGPSVFLFPPKPKDTLMISRTPE |
| | | VTCVVVDVSHEDPEVKFNWYVDGVEVHNAKTKPCEEQYGSTYRCVSVLTVLHQ |
| | | DWLNGKEYKCKVSNKALPAPIEKTISKAKGQPREPQVYTLPPSREEMTKNQVSLT |
| | | CLVKGFYPSDIAVEWESNGQPENNYKTTPPVLDSDGSFFLYSKLTVDKSRWQQG |
| | | NVFSCSVMHEALHNHYTQKSLSLSPGKGGGGSGGGGSGGGGSGGGGSGGGGSG |
| | | GGGSDKTHTCPPCPAPELLGGPSVFLFPPKPKDTLMISRTPEVTCVVVDVSHEDPE |
| | | VKFNWYVDGVEVHNAKTKPCEEQYGSTYRCVSVLTVLHQDWLNGKEYKCKVS |
| | | NKALPAPIEKTISKAKGQPREPQVYTLPPSREEMTKNQVSLTCLVKGFYPSDIAVE |
| | | WESNGQPENNYKTTPPVLDSDGSFFLYSKLTVDKSRWQQGNVFSCSVMHEALHN |
| | | HYTQKSLSLSPGK |
| scp-18 | 157 | QVQLVESGGGLVKPGESLRLSCAASGFTFSDYYMYWVRQAPGKCLEWVAIISDG |
| | | GYYTYYSDIIKGRFTISRDNAKNSLYLQMNSLKAEDTAVYYCARGFPLLRHGAM |
| | | DYWGQGTLVTVSSGGGGSGGGGSGGGGSDIQMTQSPSSLSASVGDRVTITCKAS |
| | | QNVDTNVAWYQQKPGQAPKSLIYSASYRYSDVPSRFSGSASGTDFTLTISSVQSE |
| | | DFATYYCQQYDSYPYTFGCGTKLEIKSGGGGSEVQLVESGGGLVQPGGSLKLSC |
| | | AASGFTFNSYAMNWVRQAPGKGLEWVARIRSKYNNYATYYADSVKGRFTISRD |
| | | DSKNTAYLQMNNLKTEDTAVYYCVRHGNFGNSYVSWWAYWGQGTLVTVSSG |
| | | GGGSGGGGGGGGSQTVVTQEPSLTVSPGGTVTLTCGSSTGAVTSGNYPNWVQQ |
| | | KPGQAPRGLIGGTKFLAPGTPARFSGSLLGGKAALTLSGVQPEDEAEYYCVLWYS |
| | | NRWVFGGGTKLTVLGGGGDKTHTCPPCPAPELLGGPSVFLFPPKPKDTLMISRTP |
| | | EVTCVVVDVSHEDPEVKFNWYVDGVEVHNAKTKPCEEQYGSTYRCVSVLTVLH |
| | | QDWLNGKEYKCKVSNKALPAPIEKTISKAKGQPREPQVYTLPPSREEMTKNQVSL |
| | | TCLVKGFYPSDIAVEWESNGQPENNYKTTPPVLDSDGSFFLYSKLTVDKSRWQQ |
| | | GNVFSCSVMHEALHNHYTQKSLSLSPGKGGGGSGGGGSGGGGSGGGGSGGGGS |
| | | GGGGSDKTHTCPPCPAPELLGGPSVFLFPPKPKDTLMISRTPEVTCVVVDVSHEDP |
| | | EVKFNWYVDGVEVHNAKTKPCEEQYGSTYRCVSVLTVLHQDWLNGKEYKCKV |
| | | SNKALPAPIEKTISKAKGQPREPQVYTLPPSREEMTKNQVSLTCLVKGFYPSDIAV |
| | | EWESNGQPENNYKTTPPVLDSDGSFFLYSKLTVDKSRWQQGNVFSCSVMHEALH |
| | | NHYTQKSLSLSPGK |

In certain embodiments, the PSMA-targeted T-cell engaging molecule administered to a patient according to the methods of the invention comprises an amino acid sequence selected from SEQ ID NOs: 140-157. In one embodiment, the PSMA-targeted T-cell engaging molecule comprises the amino acid sequence of SEQ ID NO: 141. In another embodiment, the PSMA-targeted T-cell engaging molecule comprises the amino acid sequence of SEQ ID NO: 144. In yet another embodiment, the PSMA-targeted T-cell engaging molecule comprises the amino acid sequence of SEQ ID NO: 147. In still another embodiment, the PSMA-targeted T-cell engaging molecule comprises the amino acid sequence of SEQ ID NO: 150. In a preferred embodiment, the PSMA-targeted T-cell engaging molecule used in the methods of the invention comprises the amino acid sequence of SEQ ID NO: 140 (e.g. acapatamab).

The PSMA-targeted T-cell engaging molecules employed in the methods of the invention may be variants of the single chain polypeptides shown in Table 6 and comprise an amino acid sequence that is at least 90%, 91%, 92%, 93%, 94%, 95%, 96%, 97%, 98% or 99% identical to an amino acid sequence of SEQ ID NOs: 140-157. In one embodiment, the PSMA-targeted T-cell engaging molecule comprises an amino acid sequence that is at least 95% identical to an amino acid sequence selected from SEQ ID NOs: 140-157. In another embodiment, the PSMA-targeted T-cell engaging molecule comprises an amino acid sequence that is at least 98% identical to an amino acid sequence selected from SEQ ID NOs: 140-157. In certain embodiments, the sequence variability occurs in the peptide linker regions and/or the single-chain Fc domain.

In other embodiments, the PSMA-targeted T-cell engaging molecules used in the methods of the invention comprise two or more polypeptide chains. For instance, in some embodiments, the PSMA-targeted T-cell engaging molecules comprise three polypeptide chains, wherein the first polypeptide is a light chain from an anti-CD3 antibody, the second polypeptide is a heavy chain from an anti-CD3 antibody, and the third polypeptide is a modified heavy chain comprising a heavy chain variable region from an anti-PSMA heavy chain only antibody. Such three-polypeptide chain T-cell engaging molecules are described in WO 2020/206330, which is hereby incorporated by reference in its entirety. Accordingly, in certain embodiments, the PSMA-targeted T-cell engaging molecules used in the methods of the invention comprise:
(a) a first polypeptide comprising or consisting of a light chain polypeptide from an anti-CD3 antibody;
(b) a second polypeptide comprising or consisting of a heavy chain polypeptide from an anti-CD3 antibody; and
(c) a third polypeptide comprising a heavy chain variable region from an anti-PSMA antibody fused to a heavy chain immunoglobulin constant region.
   Exemplary polypeptide sequences for constructing a multi-chain PSMA-targeted T-cell engaging molecule are set forth in Table 7. In some such embodiments, the first polypeptide comprises the sequence of SEQ ID NO: 185, the second polypeptide comprises the sequence of SEQ ID NO: 186, and the third polypeptide comprises a sequence selected from SEQ ID NOs: 187-192. In one particular embodiment, the PSMA-targeted T-cell engaging molecule employed in the methods of the invention comprises a first polypeptide comprising the sequence of SEQ ID NO: 185, a second polypeptide comprising the sequence of SEQ ID NO: 186, and a third polypeptide comprising the sequence of SEQ ID NO: 187. In another particular embodiment, the PSMA-targeted T-cell engaging molecule employed in the methods of the invention comprises a first polypeptide comprising the sequence of SEQ ID NO: 185, a second polypeptide comprising the sequence of SEQ ID NO: 186, and a third polypeptide comprising the sequence of SEQ ID NO: 191. In yet another particular embodiment, the PSMA-targeted T-cell engaging molecule used in the methods of the invention comprises a first polypeptide comprising the sequence of SEQ ID NO: 185, a second polypeptide comprising the sequence of SEQ ID NO: 186, and a third polypeptide comprising the sequence of SEQ ID NO: 192.

TABLE 7
Exemplary Polypeptide Chains for Multi-Chain T-Cell Engaging Molecules

| | SEQ ID | |
|---|---|---|
| Description | NO. | Amino Acid Sequence |
| Anti-CD3 full | 185 | EIVMTQSPATLSVSPGERATLSCRASQSVSSNLAWYQQKPGQAPRLLIYGASTRA |
| length light | | TGIPARFSGSGSGTEFTLTISSLQSEDFAVYYCQQYNNWPWTFGQGTKVEIKRTVA |
| chain | | APSVFIFPPSDEQLKSGTASVVCLLNNFYPREAKVQWKVDNALQSGNSQESVTEQ |
| | | DSKDSTYSLSSTLTLSKADYEKHKVYACEVTHQGLSSPVTKSFNRGEC |
| Anti-CD3 full | 186 | EVQLVESGGGLVQPGRSLRLSCAASGFTEDDYAMHWVRQAPGKGLEWVSGISW |
| length heavy | | NSGSIGYADSVKGRFTISRDNAKNSLYLQMNSLRAEDTALYYCAKDSRGYGDYR |
| chain | | LGGAYWGQGTLVTVSSASTKGPSVFPLAPCSRSTSESTAALGCLVKDYFPEPVTV |
| | | SWNSGALTSGVHTFPAVLQSSGLYSLSSVVTVPSSSLGTKTYTCNVDHKPSNTKV |
| | | DKRVESKYGPPCPPCPAPEAAGGPSVFLFPPKPKDTLMISRTPEVTCVVVDVSQED |
| | | PEVQFNWYVDGVEVHNAKTKPREEQFNSTYRVVSVLTVLHQDWLNGKEYKCK |
| | | VSNKGLPSSIEKTISKAKGQPREPQVYTLPPSQEEMTKNQVSLWCLVKGFYPSDIA |
| | | VEWESNGQPENNYKTTPPVLDSDGSFFLYSRLTVDKSRWQEGNVFSCSVMHEAL |
| | | HNHYTQKSLSLSLGK |
| Anti-PSMA | 187 | QLQLQESGPGLVKPSETLSLTCTVSGGSISSSNYFWGWIRQSPGKGLEWIGSIDYS |
| full length | | GYTYYNPSLKSRVTISVDTSKNQFSLKLSSVTAADTAVYNCARHKAATADFDYR |
| heavy chain | | GQGTLVTVSSESKYGPPCPPCPAPEAAGGPSVFLFPPKPKDTLMISRTPEVTCVVV |
| #1 | | DVSQEDPEVQFNWYVDGVEVHNAKTKPREEQFNSTYRVVSVLTVLHQDWLNGK |
| | | EYKCKVSNKGLPSSIEKTISKAKGQPREPQVYTLPPSQEEMTKNQVSLSCAVKGF |
| | | YPSDIAVEWESNGQPENNYKTTPPVLDSDGSFFLVSRLTVDKSRWQEGNVFSCSV |
| | | MHEALHNHYTQKSLSLSLGK |
| Anti-PSMA | 188 | QLQLQESGPGLVKPSETLSLTCTVSGGSISSSNYFWGWIRQSPGKGLEWIGSIDYS |
| full length | | GYTYYNPSLKSRVTISVDTSKNQFSLKLSSVTAADTAVYNCARHKAATADFDYR |
| heavy chain | | GQGTLVTVSSGGGGSGGGGSQLQLQESGPGLVKPSETLSLTCTVSGGSISSSNYF |
| #2 | | WGWIRQSPGKGLEWIGSIDYSGYTYYNPSLKSRVTISVDTSKNQFSLKLSSVTAA |
| | | DTAVYNCARHKAATADEDYRGQGTLVTVSSESKYGPPCPPCPAPEAAGGPSVFLF |
| | | PPKPKDTLMISRTPEVTCVVVDVSQEDPEVQFNWYVDGVEVHNAKTKPREEQFN |
| | | STYRVVSVLTVLHQDWLNGKEYKCKVSNKGLPSSIEKTISKAKGQPREPQVYTLP |
| | | PSQEEMTKNQVSLSCAVKGFYPSDIAVEWESNGQPENNYKTTPPVLDSDGSFFLV |
| | | SRLTVDKSRWQEGNVFSCSVMHEALHNHYTQKSLSLSLGK |
| Anti-PSMA | 189 | QVQLVESGGGVVQPGRSLRLSCAASGFSFSRYGMHWVRQAPGKGLEGVAVIWY |
| full length | | DGSNRYYADSVKGRFTISRDNSKNTLYLQMNSLRAEDTAVYYCAREPRIGYYYE |
| heavy chain | | SSGYYSLDYRGQGTLVTVSSESKYGPPCPPCPAPEAAGGPSVFLFPPKPKDTLMIS |
| #3 | | RTPEVTCVVVDVSQEDPEVQFNWYVDGVEVHNAKTKPREEQFNSTYRVVSVLT |
| | | VLHQDWLNGKEYKCKVSNKGLPSSIEKTISKAKGQPREPQVYTLPPSQEEMTKNQ |
| | | VSLSCAVKGFYPSDIAVEWESNGQPENNYKTTPPVLDSDGSFFLVSRLTVDKSRW |
| | | QEGNVFSCSVMHEALHNHYTQKSLSLSLGK |
| Anti-PSMA | 190 | QVQLVESGGGVVQPGRSLRLSCAASGFSFSRYGMHWVRQAPGKGLEGVAVIWY |
| full length | | DGSNRYYADSVKGRFTISRDNSKNTLYLQMNSLRAEDTAVYYCAREPRIGYYYE |
| heavy chain | | SSGYYSLDYRGQGTLVTVSSGGGGSGGGGSQVQLVESGGGVVQPGRSLRLSCAA |
| #4 | | SGFSFSRYGMHWVRQAPGKGLEGVAVIWYDGSNRYYADSVKGRFTISRDNSKN |
| | | TLYLQMNSLRAEDTAVYYCAREPRIGYYYESSGYYSLDYRGQGTLVTVSSESKY |
| | | GPPCPPCPAPEAAGGPSVFLFPPKPKDTLMISRTPEVTCVVVDVSQEDPEVQFNWY |
| | | VDGVEVHNAKTKPREEQFNSTYRVVSVLTVLHQDWLNGKEYKCKVSNKGLPSSI |
| | | EKTISKAKGQPREPQVYTLPPSQEEMTKNQVSLSCAVKGFYPSDIAVEWESNGQP |
| | | ENNYKTTPPVLDSDGSFFLVSRLTVDKSRWQEGNVFSCSVMHEALHNHYTQKSL |
| | | SLSLGK |
| Anti-PSMA | 191 | QLQLQESGPGLVKPSETLSLTCTVSGGSISSSNYFWGWIRQSPGKGLEWIGSIDYS |
| full length | | GYTYYNPSLKSRVTISVDTSKNQFSLKLSSVTAADTAVYNCARHKAATADFDYR |
| heavy chain | | GQGTLVTVSSGGGGSGGGGSQVQLVESGGGVVQPGRSLRLSCAASGFSFSRYGM |
| #5 | | HWVRQAPGKGLEGVAVIWYDGSNRYYADSVKGRFTISRDNSKNTLYLQMNSLR |
| | | AEDTAVYYCAREPRIGYYYESSGYYSLDYRGQGTLVTVSSESKYGPPCPPCPAPE |
| | | AAGGPSVFLFPPKPKDTLMISRTPEVTCVVVDVSQEDPEVQFNWYVDGVEVHNA |
| | | KTKPREEQFNSTYRVVSVLTVLHQDWLNGKEYKCKVSNKGLPSSIEKTISKAKGQ |
| | | PREPQVYTLPPSQEEMTKNQVSLSCAVKGFYPSDIAVEWESNGQPENNYKTTPPV |
| | | LDSDGSFFLVSRLTVDKSRWQEGNVFSCSVMHEALHNHYTQKSLSLSLGK |
| Anti-PSMA | 192 | QVQLVESGGGVVQPGRSLRLSCAASGFSFSRYGMHWVRQAPGKGLEGVAVIWY |
| full length | | DGSNRYYADSVKGRFTISRDNSKNTLYLQMNSLRAEDTAVYYCAREPRIGYYYE |
| heavy chain | | SSGYYSLDYRGQGTLVTVSSGGGGSGGGGSQLQLQESGPGLVKPSETLSLTCTVS |
| #6 | | GGSISSSNYFWGWIRQSPGKGLEWIGSIDYSGYTYYNPSLKSRVTISVDTSKNQFS |
| | | LKLSSVTAADTAVYNCARHKAATADFDYRGQGTLVTVSSESKYGPPCPPCPAPE |
| | | AAGGPSVFLFPPKPKDTLMISRTPEVTCVVVDVSQEDPEVQFNWYVDGVEVHNA |
| | | KTKPREEQFNSTYRVVSVLTVLHQDWLNGKEYKCKVSNKGLPSSIEKTISKAKGQ |
| | | PREPQVYTLPPSQEEMTKNQVSLSCAVKGFYPSDIAVEWESNGQPENNYKTTPPV |
| | | LDSDGSFFLVSRLTVDKSRWQEGNVFSCSVMHEALHNHYTQKSLSLSLGK |

In certain embodiments, the methods of the invention comprise administering to a patient in need of treatment for prostate cancer one or more cycles of an anti-androgen compound in combination with a PSMA-targeted T-cell engaging molecule, wherein the first cycle is about 28 days and comprises:
administering a first dose (e.g. a priming dose) of about 30 μg to about 300 μg of the T-cell engaging molecule by continuous intravenous infusion over days 1 to 3 of the cycle;
administering a therapeutic dose of about 90 μg to about 1.8 mg of the T-cell engaging molecule by a bolus intravenous infusion on days 8 and 22 of the cycle; and
administering an anti-androgen compound orally once per day on each of days 15 to 28 of the cycle.

In some embodiments of the methods of the invention, the first cycle is about 28 days and comprises:
administering a first dose (e.g. a priming dose) of about 90 μg of the T-cell engaging molecule by continuous intravenous infusion over days 1 to 3 of the cycle;
administering a therapeutic dose of about 150 μg or about 300 μg of the T-cell engaging molecule by a bolus intravenous infusion on days 8 and 22 of the cycle; and
administering an anti-androgen compound orally once per day on each of days 15 to 28 of the cycle.

In any of the foregoing embodiments, the PSMA-targeted T-cell engaging molecule can be acapatamab, which is a single chain polypeptide comprising the sequence of SEQ ID NO: 140, and the anti-androgen compound can be enzalutamide, abiraterone, abiraterone acetate, darolutamide, or apalutamide. For instance, in some embodiments, the methods of the invention comprise administering to the patient in need of treatment for prostate cancer one or more cycles of enzalutamide in combination with acapatamab, wherein the first cycle is about 28 days and comprises:
administering a first dose (e.g. a priming dose) of about 90 μg of acapatamab by continuous intravenous infusion over days 1 to 3 of the cycle;
administering a therapeutic dose of about 150 μg or about 300 μg of acapatamab by a bolus intravenous infusion on days 8 and 22 of the cycle; and
administering enzalutamide orally at a dose of about 160 mg once per day on each of days 15 to 28 of the cycle.

In other embodiments, the methods of the invention comprise administering to the patient in need of treatment for prostate cancer one or more cycles of abiraterone or abiraterone acetate in combination with acapatamab, wherein the first cycle is about 28 days and comprises:
administering a first dose (e.g. a priming dose) of about 90 μg of acapatamab by continuous intravenous infusion over days 1 to 3 of the cycle;
administering a therapeutic dose of about 150 μg or about 300 μg of acapatamab by a bolus intravenous infusion on days 8 and 22 of the cycle; and
administering abiraterone or abiraterone acetate orally at a dose of about 1,000 mg once per day on each of days 15 to 28 of the cycle.

In certain embodiments, the methods of the invention comprise administering to the patient in need of treatment for prostate cancer one or more cycles of darolutamide in combination with acapatamab, wherein the first cycle is about 28 days and comprises:
administering a first dose (e.g. a priming dose) of about 90 μg of acapatamab by continuous intravenous infusion over days 1 to 3 of the cycle;
administering a therapeutic dose of about 150 μg or about 300 μg of acapatamab by a bolus intravenous infusion on days 8 and 22 of the cycle, and
administering darolutamide orally at a dose of about 600 mg twice per day on each of days 15 to 28 of the cycle.

In certain other embodiments, the methods of the invention comprise administering to the patient in need of treatment for prostate cancer one or more cycles of apalutamide in combination with acapatamab, wherein the first cycle is about 28 days and comprises:
administering a first dose (e.g. a priming dose) of about 90 μg of acapatamab by continuous intravenous infusion over days 1 to 3 of the cycle;
administering a therapeutic dose of about 150 μg or about 300 μg of acapatamab by a bolus intravenous infusion on days 8 and 22 of the cycle; and
administering apalutamide orally at a dose of about 240 mg once per day on each of days 15 to 28 of the cycle.

In any of the foregoing embodiments, the methods may further comprise administering to the patient a maintenance cycle, wherein the maintenance cycle comprises administering the therapeutic dose of the PSMA-targeted T-cell engaging molecule (e.g. acapatamab) by a bolus intravenous infusion once every 14 days (Q2W) and administering the anti-androgen compound orally once per day or twice per day on each day of the cycle.

In certain embodiments of the methods of the invention, one or more premedications can be administered to the patient prior to the administration of a first dose of a PSMA-targeted T-cell engaging molecule in the first cycle. In some embodiments, the premedication is administered to the patient prior to administration of each dose of the PSMA-targeted T-cell engaging molecule in the first cycle. The premedication may also be administered to the patient prior to administration of one or more doses of the PSMA-targeted T-cell engaging molecule in one or more maintenance cycles. In some embodiments, the premedication is only administered to the patient prior to administration of one or more doses during the first cycle and is not administered to the patient prior to administration of any dose of the PSMA-targeted T-cell engaging molecule in a subsequent treatment cycle (e.g. a maintenance cycle). It is envisaged that “prior to,” in this specific context, means within 72 hours, 48 hours, 36, hours, 24 hours, 18 hours, 16 hours, 12 hours, 6 hours, 5 hours, 4 hours, or 3 hours, and preferably within 120, 90, 60 or 30 minutes before the start of administration of the PSMA-targeted T-cell engaging molecule. Depending on the type of premedication used and the route by which it is administered, the premedication may, for example, be administered 30-120 or 30-60 minutes prior to start of administration of the PSMA-targeted T-cell engaging molecule. The premedication may be administered, e.g. to prevent or reduce severity of infusion-related reactions and/or to prevent or reduce severity of cytokine release syndrome or its symptoms.

In some embodiments, the premedication is an antihistamine. The antihistamine can be administered orally or intravenously and can be administered at a dose equivalent to diphenhydramine 50 mg i.v. Suitable antihistamines that can be administered as a premedication include, but are not limited to, antihistamines of oral, parenteral or rectal route such as: azatadine (maximum dose e.g. 4 mg/day), brompheniramine (maximum dose e.g. 30 mg/day), cetirizine (maximum dose e.g. 15 mg/day), chlorpheniramine (maximum dose e.g. 30 mg/day), clemastine (maximum dose e.g. 10 mg/day), cyproheptadine (maximum dose e.g. 15 mg/day), desloratadine (maximum dose e.g. 7 mg/day), dexchlorpheniramine (maximum dose e.g. 15 mg/day), diphenhydramine (maximum dose e.g. 350 mg/per day), doxylamine (maximum dose e.g. 180 mg/day), fexofenadine (maximum dose e.g. 200 mg/day), loratadine (maximum dose e.g. 15 mg/day), and phenindamine (maximum dose e.g. 180 mg/day).

In other embodiments, the premedication is a glucocorticoid. Glucocorticoids are a class of corticosteroids, which are a class of steroid hormones. Glucocorticoids are corticosteroids that bind to the glucocorticoid receptor. A less common synonym is glucocorticosteroid. Cortisol (known as hydrocortisone when used as a medication) is the most important human glucocorticoid. A variety of synthetic glucocorticoids, some far more potent than cortisol, have been created for therapeutic use. Cortisol is the standard of comparison for glucocorticoid potency. One example for commonly prescribed replacement steroid equivalents may be prednisone (5 mg)=cortisone (25 mg)=dexamethasone (0.75 mg)=hydrocortisone (20 mg)=methylprednisolone (4 mg). These doses indicate the equivalent pharmacologic dose of systemic glucocorticoids. The glucocorticoid can be administered orally or intravenously and can be administered at a dose equivalent to 4-20 mg dexamethasone i.v. (the equivalence referring to the glucocorticoid potency). The dose of glucocorticoid can be the same at each administration (i.e. at each time the glucocorticoid premedication is administered). Alternatively, the dose of glucocorticoid can be reduced in subsequent administrations, e.g. by 50% of the previous dose, if there are no or minimal signs of infusion reactions and/or CRS symptoms following the previous administration of the PSMA-targeted T-cell engaging molecule. In certain embodiments, glucocorticoids are only administered as premedications during the first cycle and are not administered in subsequent treatment cycles (e.g. maintenance cycles).

Examples of glucocorticoids to be used as a premedication include, but are not limited to, cortisone, hydrocortisone, prednisone, prednisolone, methylprednisolone, dexamethasone, betamethasone, beclomethasone, budesonide, triamcinolone, cloprednol, deflazacort, fluocortolone, cortivazol, paramethasone, fluticasone, fluticasone propionate, triamcinolone acetonide, as well as combinations and/or pharmaceutically acceptable derivatives thereof. The different glucocorticoids may be used alone or in combination. Dexamethasone, prednisone and prednisolone are preferred glucocorticoids for use as a premedication according to the methods of the invention. In certain embodiments of the methods of the invention, the glucocorticoid administered to the patient prior to administration of one or more (or all) doses of the PSMA-targeted T-cell engaging molecule during the first cycle and/or maintenance cycle is dexamethasone. Dexamethasone can be administered at a dose of about 4-20 mg, 6-18 mg, 8-16 mg, about 16 mg, or about 8 mg at each administration. In some embodiments of the methods of the invention, dexamethasone is administered to the patient prior to the administration of each dose of the PSMA-targeted T-cell engaging molecule during the first cycle. In these and other embodiments, dexamethasone is orally administered to the patient at a dose of about 8 mg about 6-16 hours prior to administration of each dose of the PSMA-targeted T-cell engaging molecule during the first cycle. In other embodiments, dexamethasone is intravenously administered to the patient at a dose of about 8 mg within one hour prior to administration of each dose of the PSMA-targeted T-cell engaging molecule during the first cycle. In still other embodiments, the methods of the invention further comprise administering during the first cycle an 8 mg dose of dexamethasone orally (or equivalent dose of other glucocorticoid) to the patient about 6-16 hours prior to administration of each dose of the PSMA-targeted T-cell engaging molecule and administering an 8 mg dose of dexamethasone intravenously (or equivalent dose of other glucocorticoid) to the patient within one hour prior to administration of each dose of the PSMA-targeted T-cell engaging molecule.

A patient may be treated according to the methods of the invention for a set treatment period. A “treatment period” begins upon administration of a first dose of a PSMA-targeted T-cell engaging molecule in a first cycle or an initiation cycle and ends upon administration of a final dose of a PSMA-targeted T-cell engaging molecule and/or an anti-androgen compound in a maintenance cycle. The treatment period may be from about 3 months to about 36 months, from about 12 months to about 24 months, or from about 6 months to about 12 months. For instance, the treatment period may be about 3 months, about 4 months, about 5 months, about 6 months, about 7 months, about 8 months, about 9 months, about 10 months, about 11 months, about 12 months, about 13 months, about 14 months, about 15 months, about 18 months, about 21 months, about 24 months, about 27 months, about 30 months, about 33 months, or about 36 months. In some embodiments, the treatment period is about 6 months. In other embodiments, the treatment period is about 9 months. In yet other embodiments, the treatment period is about 12 months. The treatment period can be adjusted for each patient depending on the patient's response to treatment. In one particular embodiment, the patient is treated according to the methods of the invention until the patient achieves a complete response or until evidence of prostate cancer is otherwise undetectable in the patient.

In some embodiments, the patients to be treated according to the methods of the invention may have failed or be intolerant to one or more prior prostate cancer therapies, such as chemotherapy, radiation therapy, androgen deprivation therapy, or radioligand therapy. As used herein, a patient may be considered to have failed a therapy if the patient's cancer progresses (e.g. size of prostate tumors increases; an increase in the presence, number or size of metastatic lesions; elevations in blood levels of PSA) following a standard regimen of the therapy. A patient may also be considered to have failed a therapy if the patient is unable to tolerate the therapy or the therapy is contraindicated in the patient. As the terms are used herein, a patient is considered to be refractory or resistant to a therapy if the patient's cancer does not respond or loses an initial response following continued administration of the therapy. As used herein, a patient is considered to have relapsed after a therapy if the signs and symptoms of prostate cancer (e.g. elevation of blood PSA levels, cancerous cells in prostate gland, appearance of metastatic lesions, etc.) return after the patient has experienced a remission from the disease.

In certain embodiments, the patients to be treated according to the methods of the invention have failed or are intolerant to one or more chemotherapy regimens. In related embodiments, the patients to be treated according to the methods of the invention are refractory or resistant to one or more chemotherapy regimens. Standard chemotherapy regimens for treating prostate cancer typically include regimens of mitoxantrone, estramustine, carboplatin, oxaliplatin, cisplatin, and taxane chemotherapy regimens, for example regimens with docetaxel, cabazitaxel, or paclitaxel. In one embodiment, the patient to be treated according to the methods of the invention has failed or is intolerant, refractory, or resistant to one or more taxane chemotherapy regimens. In another embodiment, the patient to be treated according to the methods of the invention has failed or is intolerant, refractory, or resistant to two or more taxane chemotherapy regimens. In such embodiments, the patient may have failed or is intolerant, refractory, or resistant to a docetaxel regimen and/or a cabazitaxel regimen.

In other embodiments, the patients to be treated according to the methods of the invention have failed or are intolerant, refractory or resistant to one or more androgen deprivation therapies. Androgen deprivation therapy includes, but is not limited to, surgical castration (e.g. bilateral orchiectomy), chemical castration with LHRH agonists or antagonists (e.g. leuprolide, goserelin, triptorelin, histrelin, or degarelix), or treatment with anti-androgen compounds, such as androgen biosynthesis inhibitors (e.g. abiraterone, ketoconazole), or androgen receptor antagonists (e.g. flutamide, bicalutamide, nilutamide, enzalutamide, apalutamide, or darolutamide). In certain embodiments, the patient has failed or is intolerant, resistant or refractory to at least one prior anti-androgen compound, such as abiraterone, ketoconazole, flutamide, bicalutamide, nilutamide, enzalutamide, apalutamide, or darolutamide. In related embodiments, the patient has failed or is intolerant, resistant or refractory to one or more anti-androgen compounds selected from abiraterone, enzalutamide, apalutamide, and darolutamide. In such embodiments where the patient has failed or is intolerant, resistant or refractory to treatment with a prior anti-androgen compound, the anti-androgen compound administered to the patient according to the combination therapy methods of the invention is preferably a different anti-androgen compound than the one the patient received previously.

In certain other embodiments, the patients to be treated according to the methods of the invention have failed or are intolerant, refractory or resistant to a radioligand therapy. A radioligand therapy is an agent that comprises a radionuclide or radioactive isotope covalently attached to a targeting ligand (e.g. an antibody, peptide, or small molecule) that specifically binds to a protein on the surface of a cancer cell. In some embodiments, the radioligand therapy to which the patient is refractory or resistant is a PSMA-targeted radioligand therapy, for example comprising a radionuclide (e.g. lutetium-177 (¹⁷⁷Lu), actinium-225 (²²⁵Ac), yttrium-90 (⁹⁰Y), or iodine-131 (¹³¹I)) attached to a PSMA-targeted ligand, such as PSMA-11, PSMA-617, PSMA-1007, an anti-PSMA antibody (e.g. humanized antibody J591) or binding fragment thereof, or MIP-1095 ((S)-2-(3-((S)-1-carboxy-5-(3-(4-iodophenyl) ureido) pentyl) ureido) pentanedioic acid). PSMA-targeted radiotherapies include, but are not limited to, ¹⁷⁷Lu-PSMA-617, ²²⁵Ac-PSMA-617, ²²⁵Ac-huJ591, ¹⁷⁷Lu-huJ591, ⁹⁰Y-huJ591, and ¹³¹I-MIP-1095. Other PSMA-targeted radiotherapies are described in Czerwinksa et al., Molecules, Vol. 25:1743, 2020, which is hereby incorporated by reference in its entirety. In one embodiment, the patients to be treated according to the methods of the invention have failed or are intolerant, refractory or resistant to a ¹⁷⁷Lu-PSMA-617 radioligand therapy. In another embodiment, the patients to be treated according to the methods of the invention have failed or are intolerant, refractory or resistant to a ²²⁵Ac-PSMA-617 radioligand therapy.

The PSMA-targeted T-cell engaging molecules for use in the methods of the invention may be prepared by any number of conventional techniques. For example, the PSMA-targeted T-cell engaging molecules described herein may be produced by recombinant expression systems, using any technique known in the art. See, e.g., Monoclonal Antibodies, Hybridomas: A New Dimension in Biological Analyses, Kennet et al. (eds.) Plenum Press, New York (1980); and Antibodies: A Laboratory Manual, Harlow and Lane (eds.), Cold Spring Harbor Laboratory Press, Cold Spring Harbor, N.Y. (1988).

PSMA-targeted T-cell engaging molecules or components thereof (e.g. Fv fragments, Fc monomers) can be expressed in hybridoma cell lines or in cell lines other than hybridomas. Expression vectors or constructs encoding the T-cell engaging molecules can be used to transform a mammalian, insect or microbial host cell. The term “vector” refers to any molecule or entity (e.g., nucleic acid, plasmid, bacteriophage or virus) used to transfer protein coding information into a host cell. Examples of vectors include, but are not limited to, plasmids, viral vectors, non-episomal mammalian vectors and expression vectors, for example, recombinant expression vectors. The term “expression vector” or “expression construct” as used herein refers to a recombinant nucleic acid molecule containing a desired coding sequence and appropriate nucleic acid control sequences necessary for the expression of the operably linked coding sequence in a particular host cell. An expression vector can include, but is not limited to, sequences that affect or control transcription, translation, and, if introns are present, affect RNA splicing of a coding region operably linked thereto. Nucleic acid sequences necessary for expression in prokaryotes include a promoter, optionally an operator sequence, a ribosome binding site and possibly other sequences Eukaryotic cells are known to utilize promoters, enhancers, and termination and polyadenylation signals. A secretory signal peptide sequence can also, optionally, be encoded by the expression vector, operably linked to the coding sequence of interest, so that the expressed polypeptide can be secreted by the recombinant host cell, for more facile isolation of the polypeptide of interest from the cell, if desired.

Recombinant expression vectors or constructs will typically comprise a nucleic acid molecule encoding a polypeptide comprising one or more of the following: one or more CDRs provided herein; a light chain constant region; a light chain variable region; a heavy chain constant region (e.g., CHI, CH2 and/or CH3); a heavy chain variable region; hinge region, Fc domain, and/or another scaffold portion of an anti-PSMA antibody or anti-CD3 antibody. These nucleic acid sequences are inserted into an appropriate expression vector using standard ligation techniques. In embodiments in which the T-cell engaging molecule is a single chain polypeptide or single chain fusion protein, the nucleic acid comprised in the recombinant expression vector will typically encode the full-length single chain polypeptide (e.g. full-length single chain fusion protein). The vector is typically selected to be functional in the particular host cell employed (i.e., the vector is compatible with the host cell machinery, permitting amplification and/or expression of the gene can occur). In some embodiments, vectors are used that employ protein-fragment complementation assays using protein reporters, such as dihydrofolate reductase (see, for example, U.S. Pat. No. 6,270,964, which is hereby incorporated by reference). Suitable expression vectors can be purchased, for example, from Invitrogen Life Technologies or BD Biosciences (formerly “Clontech”). Other useful vectors for cloning and expressing the T-cell engaging molecules and components thereof include those described in Bianchi and McGrew, 2003, Biotech. Biotechnol. Bioeng. 84:439-44, which is hereby incorporated by reference. Additional suitable expression vectors are discussed, for example, in Methods Enzymol., vol. 185 (D. V. Goeddel, ed.), 1990, New York: Academic Press.

Typically, expression vectors used in any of the host cells to produce a PSMA-targeted T-cell engaging molecule will contain sequences for cloning and expression of exogenous nucleotide sequences encoding the T-cell engaging molecule or components thereof. Such sequences, collectively referred to as “flanking sequences,” in certain embodiments will typically include one or more of the following nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element.

Optionally, the vector may contain a “tag”-encoding sequence, i.e., an oligonucleotide molecule located at the 5′ or 3′ end of the PSMA-targeted T-cell engaging molecule coding sequence; the oligonucleotide sequence encodes polyHis (such as hexaHis), or another “tag” such as FLAG® tag, HA (hemaglutinin influenza virus), or myc, for which commercially available antibodies exist. This tag is typically fused to the polypeptide upon expression of the polypeptide and can serve as a means for affinity purification or detection of the PSMA-targeted T-cell engaging molecule from the host cell. Affinity purification can be accomplished, for example, by column chromatography using antibodies against the tag as an affinity matrix. Optionally, the tag can subsequently be removed from the purified T-cell engaging molecule by various means such as using certain peptidases for cleavage.

Expression and cloning vectors will typically contain a promoter that is recognized by the host cell and operably linked to the nucleic acid molecule encoding a PSMA-targeted T-cell engaging molecule. The term “operably linked” as used herein refers to the linkage of two or more nucleic acid sequences in such a manner that a nucleic acid molecule capable of directing the transcription of a given gene and/or the synthesis of a desired protein molecule is produced. For example, a control sequence in a vector that is “operably linked” to a protein coding sequence is ligated thereto so that expression of the protein coding sequence is achieved under conditions compatible with the transcriptional activity of the control sequences. More specifically, a promoter and/or enhancer sequence, including any combination of cis-acting transcriptional control elements is operably linked to a coding sequence if it stimulates or modulates the transcription of the coding sequence in an appropriate host cell or other expression system. A large number of promoters, recognized by a variety of potential host cells, are well known to those of skill in the art. For example, suitable promoters for use with mammalian host cells include those obtained from the genomes of viruses such as polyoma virus, fowlpox virus, adenovirus (such as Adenovirus 2), bovine papilloma virus, avian sarcoma virus, cytomegalovirus, retroviruses, hepatitis-B virus, and Simian Virus 40 (SV40). A suitable promoter is operably linked to the polynucleotide encoding e.g., a PSMA-targeted T-cell engaging molecule or component thereof, by removing the promoter from the source nucleic acid by restriction enzyme digestion and inserting the desired promoter sequence into the vector.

The expression vectors for recombinant production of the PSMA-targeted T-cell engaging molecules described herein may be constructed from a starting vector such as a commercially available vector. Such vectors may or may not contain all of the desired flanking sequences. Where one or more of the desired flanking sequences are not already present in the vector, they may be individually obtained and ligated into the vector. Methods used for obtaining each of the flanking sequences are well known to one skilled in the art. The expression vectors can be introduced into host cells to thereby produce the PSMA-targeted T-cell engaging molecules encoded by the nucleic acids present in the vectors.

After the vector has been constructed and one or more nucleic acid molecules encoding the PSMA-targeted T-cell engaging molecule or component thereof has been inserted into the proper site(s) of the vector or vectors, the completed vector(s) may be inserted into a suitable host cell for amplification and/or polypeptide expression. The term “host cell” as used herein refers to a cell that has been transformed, or is capable of being transformed, with a nucleic acid and thereby expresses a gene of interest. The term includes the progeny of the parent cell, whether or not the progeny is identical in morphology or in genetic make-up to the original parent cell, so long as the gene of interest is present. A host cell that comprises an isolated polynucleotide or nucleic acid encoding a T-cell engaging molecule, preferably operably linked to at least one expression control sequence (e.g. promoter or enhancer), is a “recombinant host cell.”

The transformation of an expression vector for a polypeptide into a selected host cell may be accomplished by well-known methods including transfection, infection, calcium phosphate co-precipitation, electroporation, microinjection, lipofection, DEAE-dextran mediated transfection, or other known techniques. The method selected will in part be a function of the type of host cell to be used.

A host cell, when cultured under appropriate conditions, synthesizes a T-cell engaging molecule that can subsequently be collected from the culture medium (if the host cell secretes it into the medium) or directly from the host cell producing it (if it is not secreted). The selection of an appropriate host cell will depend upon various factors, such as desired expression levels, polypeptide modifications that are desirable or necessary for activity (such as glycosylation or phosphorylation) and ease of folding into a biologically active molecule. Suitable host cells include, but are not limited to, prokaryotic cells (e.g. E. coli, B. subtilis), yeast cells (Saccharmoyces cerevisiae, Pichia pastoris), and mammalian cells (e.g. Chinese hamster ovary (CHO), human embryonic kidney (HEK)). CHO cells are preferred host cells in some embodiments for expressing the PSMA-targeted T-cell engaging molecules.

Host cells are transformed or transfected with the above-described expression vectors for production of the T-cell engaging molecules and are cultured in conventional nutrient media modified as appropriate for inducing promoters, selecting transformants, or amplifying the genes encoding the desired sequences. The host cells used to produce the T-cell engaging molecules may be cultured in a variety of media. Commercially available media such as Ham's F10 (Sigma), Minimal Essential Medium (MEM, Sigma), RPMI-1640 (Sigma), and Dulbecco's Modified Eagle's Medium (DMEM, Sigma) are suitable for culturing the host cells. In addition, any of the media described in Ham et al., Meth. Enz. 58:44, 1979; Barnes et al., Anal. Biochem. 102:255, 1980; U.S. Pat. Nos. 4,767,704, 4,657,866; 4,927,762; 4,560,655; or 5,122,469; WO 90/03430; or WO 87/00195 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as Gentamycin™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression and will be apparent to the ordinary skilled artisan.

Upon culturing the host cells, the T-cell engaging molecule can be produced intracellularly, in the periplasmic space, or directly secreted into the medium. If the T-cell engaging molecule is produced intracellularly, as a first step, the host cells are lysed (e.g., by mechanical shear, osmotic shock, or enzymatic methods) and the particulate debris (e.g., host cells and lysed fragments), is removed, for example, by centrifugation, microfiltration, or ultrafiltration. If the T-cell engaging molecule is secreted into the culture medium, the T-cell engaging molecule can be separated from host cells through centrifugation or microfiltration, and optionally, subsequently concentrated through ultrafiltration. The PSMA-targeted T-cell engaging molecules can be further purified or partially purified using, for example, one or more chromatography steps, such as affinity chromatography (e.g. protein A, protein L, or protein G affinity chromatography), cation exchange chromatography, anion exchange chromatography, hydroxyapatite chromatography, hydrophobic interaction chromatography, or mixed mode chromatography.

The PSMA-targeted T-cell engaging molecule and the anti-androgen compound are generally administered to the patient in a pharmaceutical composition, which can include pharmaceutically acceptable carriers, excipients, or diluents. “Pharmaceutically acceptable” refers to molecules, compounds, and compositions that are non-toxic to human recipients at the dosages and concentrations employed and/or do not produce allergic or adverse reactions when administered to humans. In certain embodiments, the pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; coatings, monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium, potassium, magnesium, calcium, acetate, hydrochloride, phosphate and the like); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. Methods and suitable materials for formulating molecules for therapeutic use are known in the pharmaceutical arts, and are described, for example, in REMINGTON'S PHARMACEUTICAL SCIENCES, 18th Edition, (A. R. Genrmo, ed.), 1990, Mack Publishing Company. Pharmaceutical compositions comprising the PSMA-targeted T-cell engaging molecules or the anti-androgen compounds to be administered according to the methods of the invention include, but are not limited to, solid, liquid, frozen, and lyophilized compositions.

If the pharmaceutical composition has been lyophilized, the lyophilized material is reconstituted in an appropriate liquid prior to administration. The lyophilized material may be reconstituted in, e.g., bacteriostatic water for injection (BWFI), physiological saline, phosphate buffered saline (PBS), or the same formulation the protein had been in prior to lyophilization.

In some embodiments, the selection of carriers and excipients for incorporation into the pharmaceutical compositions influences the physical state, stability, rate of in vivo release and rate of in vivo clearance of the T-cell engaging molecules or anti-androgen compounds. In certain embodiments, the primary vehicle or carrier in a pharmaceutical composition may be either aqueous or non-aqueous in nature. For example, a suitable vehicle or carrier for the T-cell engaging molecules may be water for injection, physiological saline solution, possibly supplemented with other materials or excipients common in compositions for parenteral administration.

In certain embodiments, the pharmaceutical compositions comprise an effective amount of the PSMA-targeted T-cell engaging molecule or an anti-androgen compound and one or more excipients. An effective amount can be a therapeutic dose or it may be a smaller amount, such as a priming dose or unit dose. Excipients can be used for a wide variety of purposes, such as adjusting physical, chemical, or biological properties of formulations, such as adjustment of viscosity, and/or to stabilize such formulations against degradation and spoilage e.g. due to stresses that occur during manufacturing, shipping, storage, pre-use preparation, and administration.

In some embodiments, the pharmaceutical composition comprising an effective amount of a PSMA-targeted T-cell engaging molecule to be administered to a patient according to the methods of the invention comprises a buffer. Buffers are used to maintain the composition at physiological pH or at a slightly lower pH, typically within a pH range from about 4.0 to about 6.5. Suitable buffers include, but are not limited to, glutamate, aspartate, acetate, Tris, citrate, histidine, succinate, and phosphate buffers. In certain embodiments, the pharmaceutical composition administered according to the methods described herein comprises a glutamate buffer, particularly L-glutamate buffer. Pharmaceutical compositions comprising a glutamate buffer can have a pH of about 4.0 to about 5.5, a pH of about 4.0 to about 4.4, or a pH of about 4.2 to about 4.8.

The pharmaceutical composition comprising an effective amount of a PSMA-targeted T-cell engaging molecule may further comprise a surfactant. The term “surfactant” as used herein refers to a substance that functions to reduce the surface tension of a liquid in which it is dissolved. Surfactants can be included in pharmaceutical compositions for a variety of purposes including, for example, to prevent or control aggregation, particle formation and/or surface adsorption in liquid formulations or to prevent or control these phenomena during the lyophilization and/or reconstitution process in lyophilized formulations. Surfactants include, for example, amphipathic organic compounds that exhibit partial solubility in both organic solvents and aqueous solutions. General characteristics of surfactants include their ability to reduce the surface tension of water, reduce the interfacial tension between oil and water and also form micelles. Surfactants that may be incorporated into the pharmaceutical compositions used in the methods of the invention include both non-ionic and ionic surfactants. Suitable non-ionic surfactants include, but are not limited to, alkyl poly (ethylene oxide), alkyl polyglucosides, such as octyl glucoside and decyl maltoside, fatty alcohols, such as cetyl alcohol and oleyl alcohol, cocamide MEA, cocamide DEA, and cocamide TEA. Specific examples of non-ionic surfactants include the polysorbates including, for example, polysorbate 20, polysorbate 28, polysorbate 40, polysorbate 60, polysorbate 65, polysorbate 80, polysorbate 81, polysorbate 85 and the like; the poloxamers including, for example, poloxamer 188, also known as poloxalkol or poly (ethylene oxide)-poly(propylene oxide), poloxamer 407 or polyethylene-polypropylene glycol and the like, and polyethylene glycol (PEG). Suitable ionic surfactants include, for example, anionic, cationic and zwitterionic surfactants. Anionic surfactants include, but are not limited to, sulfonate-based or carboxylate-based surfactants such as soaps, fatty acid salts, sodium dodecyl sulfate (SDS), ammonium lauryl sulfate and other alkyl sulfate salts. Cationic surfactants include, but are not limited to, quaternary ammonium-based surfactants such as cetyl trimethylammonium bromide (CTAB), other alkyltrimethylammonium salts, cetyl pyridinium chloride, polyethoxylated tallow amine (POEA) and benzalkonium chloride. Zwitterionic or amphoteric surfactants include, for example, dodecyl betaine, dodecyl dimethylamine oxide, cocamidopropyl betaine and coco ampho glycinate. In certain embodiments, the pharmaceutical compositions administered according to the methods described herein comprise a non-ionic surfactant. In one embodiment, the non-ionic surfactant is polysorbate 20. In another embodiment, the non-ionic surfactant is polysorbate 80.

In certain embodiments, the pharmaceutical composition comprising an effective amount of a PSMA-targeted T-cell engaging molecule further comprises a stabilizing agent. As used herein, the term “stabilizing agent” refers to an excipient that stabilizes the native conformation of the polypeptide or T-cell engaging molecule and/or prevents or reduces the physical or chemical degradation of the polypeptide or T-cell engaging molecule. Suitable stabilizing agents include, but are not limited to, polyols (e.g. sorbitol, glycerol, mannitol, xylitol, maltitol, lactitol, erythritol and threitol), sugars (e.g., fructose, glucose, glyceraldehyde, lactose, arabinose, mannose, xylose, ribose, rhamnose, galactose maltose, sucrose, trehalose, sorbose, sucralose, melezitose and raffinose), and amino acids (e.g., glycine, methionine, proline, lysine, arginine, histidine, or glutamic acid). In some embodiments, the pharmaceutical composition comprises a sugar as a stabilizing agent. In these and other embodiments, the sugar is sucrose.

Exemplary pharmaceutical compositions comprising T-cell engaging molecules, including PSMA-targeted T-cell engaging molecules, are described in WO 2018/141910, which is hereby incorporated by reference in its entirety. In certain embodiments, a pharmaceutical composition useful for the treatment of prostate cancer according to the methods described herein comprises about 0.5 mg/ml to about 2 mg/ml of a PSMA-targeted T-cell engaging molecule, about 5 mM to about 20 mM L-glutamic acid, about 0.005% to about 0.015% weight/volume (w/v) polysorbate (e.g. polysorbate 20 or polysorbate 80), and about 7% to about 12% (w/v) sucrose. In other embodiments, the pharmaceutical composition comprises about 0.5 mg/ml to about 1 mg/ml of a PSMA-targeted T-cell engaging molecule, about 8 mM to about 12 mM L-glutamic acid, about 0.008% to about 0.012% (w/v) polysorbate (e.g. polysorbate 20 or polysorbate 80), and about 8% to about 10% (w/v) sucrose. The pH of these compositions is in the range of about 4.0 to about 4.4 (e.g., pH of about 4.0, about 4.1, about 4.2, about 4.3, or about 4.4).

Any of the pharmaceutical compositions comprising the PSMA-targeted T-cell engaging molecules described herein can be lyophilized and reconstituted with, e.g. sterile water for injection, prior to administration to the patient. Reconstitution volumes will depend on the protein content following lyophilization and the desired concentration of the T-cell engaging molecule in the reconstituted solution, but may be from about 0.5 ml to about 5 ml. The solution following reconstitution can be further diluted with a diluent (e.g. saline and/or intravenous solution stabilizer (IVSS)) prior to administration to the patient as appropriate in order to administer the doses described herein according to the methods of the invention.

Any of the PSMA-targeted T-cell engaging molecules described herein, including the single chain polypeptides described in Table 6, can be incorporated into any of the pharmaceutical compositions described above and administered to a patient according to the methods described herein. In a preferred embodiment, the PSMA-targeted T-cell engaging molecule comprises the amino acid sequence of SEQ ID NO: 140 (e.g. acapatamab). In another preferred embodiment, the PSMA-targeted T-cell engaging molecule comprises the amino acid sequence of SEQ ID NO: 141.

The anti-androgen compounds administered according to the methods of the invention will typically be formulated in an oral dosage form. Oral dosage forms include, but are not limited to, tablets, pills, pellets, capsules, powders, lozenges, granules, solutions, suspensions, emulsions, syrups, elixirs, and sprays. Some oral dosage forms, such as tablets or capsules, may contain enteric coatings or films.

The following examples, including the experiments conducted and the results achieved, are provided for illustrative purposes only and are not to be construed as limiting the scope of the appended claims.

### EXAMPLES

### Example 1. A Phase 1b Study Evaluating AMG 160 in Combination with Enzalutamide in Patients with Metastatic Castration Resistant Prostate Cancer

AMG 160 (also known as acapatamab) is a half-life extended (HLE) BITE® (bispecific T-cell engager) molecule that binds both human PSMA and human CD3 and comprises a single chain IgG Fc domain. The amino acid sequence of AMG 160 is set forth in SEQ ID NO: 140. AMG 160 is designed to engage a patient's T cells to kill prostate cancer cells via binding of CD3 on T cells and PSMA on cancer cells. Enzalutamide is an androgen receptor inhibitor indicated for the treatment of patients with castration-resistant prostate cancer and, in some regions, metastatic hormone-sensitive prostate cancer. However, patients receiving enzalutamide can experience disease progression or relapse that is due in part to mutations in the androgen receptor (e.g., AR-V7) and increased androgen receptor expression, among other mechanisms (Galletti et al., Cancer Treat Rev., Vol. 57:16-27, 2017). Because these resistance mechanisms do not necessarily confer resistance to immunotherapies, like AMG 160, combination therapy of AMG 160 with enzalutamide may provide better efficacy. In addition, enzalutamide has been reported to rapidly upregulate expression of PSMA in prostate cancer cells and enhance AMG 160-mediated cytotoxicity of prostate cancer cells in vitro (see, e.g., Deegen et al, Clin. Cancer Res., Vol. 27:2928-2937, 2021; Aggarwal et al., Eur. Urol. Oncol., Vol. 1:78-82, 2018; Emmett et al., J Nucl. Med., Vol. 60:950-954, 2019). Study objectives were to evaluate safety, tolerability, and preliminary efficacy of AMG 160 in combination with enzalutamide in patients with metastatic castration-resistant prostate cancer (mCRPC).

After signing informed consent, patients entered the screening period (up to 28 days), during which eligibility of the patients was assessed. Eligible patients had mCRPC with histologically or cytologically confirmed adenocarcinoma of the prostate without pure neuroendocrine differentiation or small cell features. Patients planning to receive enzalutamide for the first time in the metastatic setting were eligible to participate in the study Specifically, patients were enrolled in the study if they met all of the following key inclusion criteria:
histologically or cytologically confirmed mCRPC without pure neuroendocrine differentiation or small cell features;
had undergone bilateral orchiectomy or were on continuous androgen-deprivation therapy (ADT) with a gonadotropin-releasing hormone (GnRH) agonist or antagonist;
had a total serum testosterone level≤50 ng/dL or 1.7 nmol/L; and
were planning to receive enzalutamide for the first time for mCRPC

Patients were excluded from the study if they: (i) had a confirmed history of or current autoimmune disease or other disease requiring permanent immunosuppressive therapy; (ii) received enzalutamide previously, (iii) had CNS metastases or leptomeningeal disease, (iv) had prior treatment with a taxane for mCRPC, (v) had major surgery and/or radiation within 4 weeks, (vi) used strong CYP2C8 inhibitors (e.g., gemfibrozil) within 7 days prior to first dose of AMG 160 or strong CYP3A4 inducers (e.g., carbamazepine, phenobarbital, phenytoin, rifabutin, rifampin, rifapentine, St John's Wart) within 28 days prior to first dose of AMG 160; or (vii) used narrow therapeutic index drugs that are substrates of CYP3A4 (e.g., alfentanil, cyclosporine, dihydroergotamine, ergotamine, fentanyl, pimozide, quinidine, sirolimus and tacrolimus), CYP2C9 (e.g., phenytoin, warfarin) or CYP2C19 (e.g., S-mephenytoin, clopidogrel) prior to first dose of AMG 160.

In the first patient cohort (cohort 1), in cycle 1, AMG 160 was administered by continuous intravenous infusion (also referred to as an extended IV (eIV) infusion) at a dose of 0.09 mg over the course of 72 hours on days 1 to 3 of a 28-day cycle, followed by a 0.3 mg therapeutic dose administered as a short IV infusion (sIV; approximately 60 minutes) on days 8 and 22 of the 28-day cycle (see Table 8 below). The initial 0.09 mg dose was administered at a constant rate over the 3-day continuous infusion period such that the total dose was delivered at a rate of 0.03 mg per day for 3 days. There was a 7-day infusion free period between cycle 1 and cycle 2 to maintain the 14-day dosing interval between the cycle 1 day 22 infusion and the cycle 2 day 1 infusion. In cycle 2 and all other subsequent cycles, AMG 160 was administered at a dose of 0.3 mg by short IV infusion (approximately 60 min) every two weeks (Q2W) in 28-day cycles on days 1 and 15. The day of the first dose of AMG 160 was defined as day 1 in each of the 28-day cycles. In cohort 1, enzalutamide was administered orally (PO) once per day (QD) at a dose of 160 mg beginning on cycle 1 day 1 (i.e. the first day of treatment with AMG 160) or up to 3 days after cycle 1 day 1.

All patients were pre-treated with 8 mg PO dexamethasone (or equivalent dose of other corticosteroids) 6-16 hours prior to all doses of AMG 160 in cycle 1. Additionally, dexamethasone 8 mg IV (or equivalent dose of other corticosteroids) was administered within 1 hour prior to all doses of AMG 160 in cycle 1. On cycle 2 day 1, 8 mg dexamethasone IV premedication was given if subjects experienced grade 2 or higher cytokine release syndrome (CRS) in cycle 1. Otherwise, dexamethasone premedication was reduced in cycle 2, if tolerated (e.g., 4-8 mg dexamethasone on cycle 2 day 1 and 0-4 mg on cycle 2 day 15). Corticosteroid ophthalmic eye drops (e.g., 1% prednisolone acetate, 2 drops per eye 4 times per day) was used for AMG 160 infusions: (1) within 6 hours prior to start of infusion, (2) during entire infusion period, and (3) for 48 hours after end of infusion in cycles 1 and 2. In cycles 3 and beyond, eye drop prophylaxis was per investigator discretion. Patients received treatment cycles of AMG 160 in combination with enzalutamide until disease progression or unacceptable toxicities.

Anti-tumor activity of AMG 160 in combination with enzalutamide was evaluated by several measures, including objective response per RECIST 1.1 criteria (as assessed by CT or MRI scans), prostate-specific antigen (PSA) response, circulating tumor cells (CTC) response, radiographic response as measured by ⁶⁸Gallium (⁶⁸Ga)-PSMA-11 (or piflufolastat F-18 (¹⁸F-DCFPyL)) positron emission tomography (PET)/computed tomography (CT) and ¹⁸F-fluorodeoxyglucose (FDG) PET/CT scans, progression-free survival (radiographic and PSA), and overall survival. CT/magnetic resonance imaging (MRI) scans were performed at baseline and every 8 weeks for the first 6 months of treatment and then every 12 weeks thereafter. Tumor burden assessments were performed based on RECIST 1.1. To confirm disease progression (PD), a second MRI/CT scan was performed at least 4 weeks after the first detection of radiographical progression. Responses (partial response (PR) and complete response (CR)) were confirmed by a repeat consecutive assessment at least 4 weeks after the first detection of radiographical response.

PSA30/50/70/90 responses were defined as 30%, 50%, 70%, and 90% reduction, respectively, in serum PSA levels, on at least two post-treatment measurements taken at least 3 weeks apart. CTC response was defined as CTC0 (reduction of CTCs>0 to 0) or CTC conversion (≥S CTCs/7.5 mL blood to ≤4 CTCs/7.5 mL blood) measured in whole blood. ⁶⁸Ga-PSMA-11 or piflufolastat F-18 (¹⁸F-DCFPyL) PET/CT scans were performed at baseline to assess PSMA-positive tumor burden and at 12 weeks and 24 weeks following treatment initiation for response assessment. To identify PSMA-negative disease burden, ¹⁸F-FDG PET/CT scans were performed at baseline.

Adverse events were assessed using the Common Terminology Criteria for Adverse Events (CTCAE), version 5.0, which is available at the web address of ctep.cancer.gov/protocol Development/electronic_applications/etc.htm, except that CRS was graded according to the criteria described in Lee et al., Blood, Vol. 124:188-195, 2014. Tumor lysis syndrome was graded according to the Cairo Bishop criteria referenced in Coiffier et al., J. Clin. Oncol., Vol. 26:2767-2778, 2008 and immune-effector cell associated neurologic syndrome (ICANS) was assessed using the criteria described in Lee et al., Biol Blood Marrow Transplant., Vol. 25:625-638, 2019.

Four patients were enrolled in cohort 1 and at the time of data analysis, all four patients were continuing therapy. Three out of four patients were evaluable for dose-limiting toxicities (DLTs). Of these 3 patients, 2 developed DLTs (grade 3 CRS and grade 3 anemia requiring transfusion). Both DLTs developed during combination therapy (i.e. after patients had received both AMG 160 and enzalutamide). All four subjects experienced grade 2 CRS, which was associated with the first or second dose of AMG 160. There were 10 serious adverse events related to the AMG 160 investigational product. All four patients exhibited a PSA90 response, all of which were confirmed responses. All four patients were assessed to have stable disease as per their on-treatment radiographic scans. None of the four patients were RECIST evaluable as none of the patients had measurable target lesions per RECIST 1.1 criteria. Non RECIST evaluable patients can only be assessed as having a complete response (CR), stable disease (SD), or progressive disease (PD).

The preliminary results from cohort I demonstrated that the combination of enzalutamide and AMG 160 at this dose level produced remarkable efficacy with all evaluable patients having a PSA90 response and positive radiographic responses (stable disease). However, the combination at this dose level with enzalutamide treatment initiated simultaneously with AMG 160 led to most patients developing DLTs. These results are somewhat surprising as overlapping toxicity of the two drugs was not expected given the difference in biological mechanisms and reported safety risks for each of the drugs. Two new cohorts (cohort 2a and cohort 2b) were opened in which the therapeutic dose of AMG 160 was reduced from 0.3 mg to 0.15 mg (see Table 8 below). In addition, in cohort 2b, the start of enzalutamide treatment was delayed until day 15 of cycle 1. The specific dosing regimens for AMG 160 and enzalutamide in cohorts 2a and 2b were as follows:
Cohort 2a
   In cycle 1, 0.09 mg of AMG 160 was administered by eIV infusion over the course of 72 hours on days 1 to 3 of a 28-day cycle, followed by a 0.15 mg therapeutic dose administered as a short IV infusion (sIV; approximately 60 minutes) on days 8 and 22 of the 28-day cycle. For cycle 2 and all other subsequent cycles, AMG 160 was administered by sIV at a dose of 0.15 mg every two weeks in 28-day cycles on days 1 and 15. There was a 7-day infusion free period between cycles 1 and 2.
   160 mg of enzalutamide was administered orally once per day beginning on cycle 1 day 1 or up to 3 days after cycle 1 day 1.
Cohort 2b
   In cycle 1, 0.09 mg of AMG 160 was administered by eIV infusion over the course of 72 hours on days 1 to 3 of a 28-day cycle, followed by a 0.15 mg therapeutic dose administered as a sIV infusion on days 8 and 22 of the 28-day cycle. For cycle 2 and all other subsequent cycles, AMG 160 was administered by sIV at a dose of 0.15 mg every two weeks in 28-day cycles on days 1 and 15. There was a 7-day infusion free period between cycles 1 and 2.
   160 mg of enzalutamide was administered orally once per day beginning on cycle 1 day 15.

Three patients were enrolled in each of cohorts 2a and 2b. As of the date of the data analysis, 2 out of 3 patients continued therapy in cohort 2a and 1 discontinued due to adverse events (grade 3 CRS and grade 3 myocardial infarction). Two out of three patients in cohort 2b continued therapy, while 1 patient discontinued due to progressive disease. In cohort 2a in which enzalutamide treatment was initiated on cycle 1 day 1 (i.e. simultaneously with AMG 160), all three patients were evaluable for DLTs and of these three patients, one patient developed a DLT (grade 3 hypoacusis) during the combination therapy period. One patient experienced grade 3 CRS, whereas the other two patients had grade 2 CRS as worst grade. There were 3 serious adverse events related to AMG 160 investigational product. All three patients had a reduction in PSA levels with 1 PSA90 response (confirmed), 1 PSA50 response (confirmed), and 1 PSA30 response (unconfirmed). At the time of data analysis, two of the three patients had on-treatment radiographic scans with one of the two patients being RECIST evaluable (i.e. having measurable target lesions at baseline). Of these two patients, one patient had a confirmed partial response and one patient had stable disease. Two of the three patients enrolled in cohort 2a had previously received treatment with another anti-androgen therapy: one patient had previously received abiraterone and the other patient had previously received abiraterone and darolutamide.

In cohort 2b in which initiation of enzalutamide treatment was delayed to cycle 1 day 15 (i.e. after the patient had received the first two doses of AMG 160, including the first therapeutic dose of AMG 160 on cycle 1 day 8), all three patients were evaluable for DLTs, but no DLTs were observed. Two of the patients experienced grade 2 CRS, whereas one patient had grade 1 CRS as worst grade. There were no serious adverse events. In terms of anti-tumor efficacy, 2 patients had confirmed PSA90 responses and 1 patient did not exhibit a PSA response. Only one patient, who was not RECIST evaluable (i.e. did not have measurable target lesions), had an on-treatment scan at the time of data analysis and had stable disease. One of three patients in cohort 2b had prior treatment with an anti-androgen therapy (apalutamide).

The results from cohorts 2a and 2b showed that delaying the start of enzalutamide treatment to the second week of cycle 1 (e.g. about 7 days following administration of the first therapeutic dose of AMG 160) significantly improved the safety profile of the combination therapy as no patients in cohort 2b experienced a DLT or serious adverse event. The severity of CRS associated with AMG 160 administration was also reduced when initiation of enzalutamide treatment was delayed. Anti-tumor efficacy was comparable between cohorts 2a and 2b with two-thirds of the patients in both cohorts exhibiting at least PSA50 responses. In view of these results, four patients were enrolled in cohort 1a in which the therapeutic dose of AMG 160 was escalated to 0.3 mg and enzalutamide treatment was initiated at cycle 1 day 15 (see Table 8 below). Preliminary results from one patient in cohort 1a, who received prior abiraterone treatment, showed that this dosing regimen produced a PSA90 (unconfirmed) response. It is expected that anti-tumor efficacy of the combination according to this dosing regimen will be similar in the other two enrolled patients and the tolerability profile will be significantly improved as compared to that observed in cohort I with fewer or no DLTs or serious adverse events observed.

TABLE 8
Summary of Cycle 1 Dosing Regimens for AMG
160 and Enzalutamide Combination Therapy

| | | Study day¹ | | | |
|---|---|---|---|---|---|
| Cohort | Drug | D 1-D 3 | D 8 | D 15 | D 22 |
| Cohort 1 | AMG 160 | 0.09 mg | 0.3 mg | — | 0.3 mg |
| | | (72 hr eIV) | sIV | | sIV |
| | Enzalutamide | 160 mg QD | | | |
| | | (C 1 D 1+)² | | | |
| Cohort 1a | AMG 160 | 0.09 mg | 0.3 mg | — | 0.3 mg |
| | | (72 hr eIV) | sIV | | sIV |
| | Enzalutamide | — | | 160 mg QD | |
| | | | | (C 1 D 15+)³ | |
| Cohort 2a | AMG 160 | 0.09 mg | 0.15 mg | — | 0.15 mg |
| | | (72 hr eIV) | sIV | | sIV |
| | Enzalutamide | 160 mg QD (C 1 D 1+)² | | | |
| Cohort 2b | AMG 160 | 0.09 mg | 0.15 mg | — | 0.15 mg |
| | | (72 hr eIV) | sIV | | sIV |

| | | |
|---|---|---|
| Enzalutamide | — | 160 mg QD |
| | | (C 1 D 15+)³ |

¹Measured from day 1 (D 1), which was the first day the patient received the first dose of AMG 160 eIV = extended IV infusion; sIV = short IV infusion (approx.. 60 minutes)
²Enzalutamide dosing was initiated on cycle 1 day 1 (C 1 D 1) or up to 3 days after cycle 1 day 1 and continued QD on each day of the cycle.
³Enzalutamide dosing was initiated on cycle 1 day 15 (C 1 D 15) and continued QD on each day of the cycle.

A second interim analysis of the data from cohorts 1, la, 2a, and 2b was conducted. A summary of the results from this second interim analysis is provided in Table 9 below. The results from this second interim analysis are consistent with those from the initial interim analysis described above. Specifically, when the initial dose of enzalutamide was delayed until after the first administration of the therapeutic dose of AMG 160 (cohorts 1a and 2b), patients had fewer serious adverse events, DLTs, and severity of CRS events as compared to when enzalutamide was initiated on the same day as the initial dose of AMG 160. When AMG 160 was administered at a therapeutic dose of 0.15 mg (cohorts 2a and 2b), PSA responses were comparable between the cohorts indicating that the delay of the start of enzalutamide treatment did not impact the anti-tumor efficacy of the combination therapy. At the 0.3 mg therapeutic dose level of AMG 160, only 2 out of the 4 patients had a PSA90 response in cohort 1a as compared to all 4 patients in cohort 1 when enzalutamide was initiated on day 1. However, all patients in cohort 1a were previously treated with at least one novel hormonal therapy (NHT), whereas 3 out of the 4 patients in cohort 1 were NHT-naïve. This could explain the lower PSA response rate in cohort 1a. Only 2 patients out of the 14 patients in all four cohorts were RECIST 1.1 evaluable at the time of data analysis. Interestingly, the only objective RECIST response was observed in cohort 1a in which AMG 160 was administered at a therapeutic dose of 0.3 mg and the initial dose of enzalutamide was delayed to day 15 of cycle 1.

Overall, the results of this study show that delaying the initial dose of enzalutamide relative to administration of the first therapeutic dose of AMG 160 improves the tolerability of the combination therapy such that higher therapeutic doses of AMG 160 can be administered to maximize anti-tumor efficacy of the combination.

TABLE 9
Summary of Interim Results of AMG 160 and
Enzalutamide Combination Therapy for mCRPC

| | AMG 160 therapeutic dose | | | |
|---|---|---|---|---|
| | 0.15 mg | | 0.30 mg | |
| | Start of enzalutamide | | | |
| | C 1 D 1 | C 1 D 15 | C 1 D 1 | C 1 D 15 |
| Cohort | Cohort 2a | Cohort 2b | Cohort 1 | Cohort 1a |
| Number of subjects | 3 | 3 | 4 | 4 |
| TEAEs (any grade)^{c} | 3 (100%) | 3 (100%) | 4 (100%) | 4 (100%) |
| TEAEs (grade ≥3)^{c} | 2 (66.7%) | 1 (33.3%) | 2 (50%) | 2 (50%) |
| SAEs^{c} | 3 (100%) | 1 (33.3%) | 3 (75%) | 1 (25%) |
| TEAEs leading to | 2 (66.7%) | 1 (33.3%) | 0 (0%) | 0 (0%) |
| discontinuation of | | | | |
| AMG 160^{c} | | | | |
| Number of subjects | 1 (33.3%) | 0 (0%) | 2 (50%) | 1 (25%)^{a} |
| with DLT | | | | |
| CRS (any grade)^{c} | 3 (100%) | 3 (100%) | 4 (100%) | 4 (100%) |
| CRS (grade ≥3)^{c} | 1 (33.3%) | 0 (0%) | 1 (25%) | 1 (25%)^{b} |
| Concomitant | | | | |
| medication use for | | | | |
| CRS treatment:^{c} | | | | |
| Oxygen | 2 (66.7%) | 0 (0%) | 3 (75%) | 2 (50%) |
| Norepinephrine | 0 (0%) | 0 (0%) | 2 (50%) | 1 (25%) |
| Dexamethasone | 0 (0%) | 0 (0%) | 2 (50%) | 3 (75%) |
| Tocilizumab | 1 (33.3%) | 1 (33.3%) | 0 (0%) | 2 (50%) |
| PSA50 response^{c} | 2 (66.7%) | 2 (66.7%) | 4 (100%) | 2 (50%) |
| PSA90 response^{c} | 1 (33.3%) | 2 (66.7%) | 4 (100%) | 2 (50%) |
| RECIST 1.1 | 0 | 1 | 0 | 1 |
| evaluable | | | | |
| ORR (%) | / | 0 (0%) | / | 1 (100%) |

^{a}Upon review, this event (grade 1 fever outside DLT period) did not qualify as a DLT and the true number of subjects with DLTs in this cohort is 0.
^{b}CRS grade 3 event occurred at C 1 D 8 prior to start of enzalutamide (i.e. outside combination therapy window)
^{c}Subject incidence for each of the indicated events
TEAE = treatment emergent adverse event;
SAE = serious adverse event;
ORR = objective response rate

### Example 2. A Phase 1b Study Evaluating AMG 160 in Combination with Abiraterone in Patients with Metastatic Castration Resistant Prostate Cancer

Abiraterone is a cytochrome P450 (CYP)17 inhibitor indicated in combination with prednisone (or prednisolone in some regions) for the treatment of patients with mCRPC and, in some regions, metastatic or high-risk castration-sensitive prostate cancer. Like enzalutamide and other NHTs, abiraterone has been reported to upregulate expression of PSMA, a target for the AMG 160 bispecific T-cell engager, on prostate cancer cells (Aggarwal et al., Eur. Urol. Oncol., Vol. 1:78-82, 2018; Emmett et al., J Nucl. Med., Vol. 60:950-954, 2019). Thus, combination therapy with AMG 160, a PSMA×CD3 T-cell engager, and abiraterone may provide synergistic anti-tumor efficacy. The primary objectives of this study were to evaluate the safety, tolerability, and preliminary efficacy of AMG 160 in combination with abiraterone in patients with mCRPC.

After signing informed consent, patients entered the screening period (up to 28 days), during which eligibility of the patients was assessed. Eligible patients had mCRPC with histologically or cytologically confirmed adenocarcinoma of the prostate without pure neuroendocrine differentiation or small cell features. Patients planning to receive abiraterone for the first time in the metastatic setting were eligible to participate in the study. Specifically, patients were enrolled in the study if they met all of the following key inclusion criteria:
histologically or cytologically confirmed mCRPC without pure neuroendocrine differentiation or small cell features;
had undergone bilateral orchiectomy or were on continuous androgen-deprivation therapy (ADT) with a gonadotropin-releasing hormone (GnRH) agonist or antagonist;
had a total serum testosterone level≤50 ng/dL or 1.7 nmol/L; and
were planning to receive abiraterone for the first time for mCRPC

Patients were excluded from the study if they: (i) had a confirmed history of or current autoimmune disease or other disease requiring permanent immunosuppressive therapy; (ii) received abiraterone previously, (iii) had CNS metastases or leptomeningeal disease, (iv) had prior treatment with a taxane for mCRPC, (v) had major surgery and/or radiation within 4 weeks, (vi) had moderate or severe hepatic impairment (Child-Pugh Class B and C) at baseline; (vii) had uncontrolled hypertension, hypokalemia, or fluid retention; (viii) had a history of or current adrenocortical insufficiency; (ix) used strong CYP3A4 inducers (e g., carbamazepine, phenobarbital, phenytoin, rifabutin, rifampin, rifapentine, St John's Wart) within 28 days prior to first dose of AMG 160; or (x) used concomitant medications that are sensitive substrates for CYP2D6 with a narrow therapeutic index (e.g., thioridazine) within 7 days prior to first dose of AMG 160.

AMG 160 (amino acid sequence set forth in SEQ ID NO: 140) was administered as described in Example 1 with the first dose of cycle 1 administered as a 72-hour extended IV infusion on days 1 to 3 followed by administration of the therapeutic dose by short IV infusion (sIV; approximately 60 minutes) on days 8 and 22 of cycle 1. Following a 7-day infusion free period between cycles 1 and 2, AMG 160 was administered by short IV infusion Q2W on days 1 and 15 of cycle 2 and all other subsequent cycles. Abiraterone was administered at a dose of 1,000 mg orally once per day beginning on cycle 1 day 15±3 days. The day of the first dose of AMG 160 was defined as day 1 in each of the 28-day cycles. Prednisone at a dose of 5 mg was administered orally twice per day (BID) on the same days the patient received abiraterone. In some regions, prednisolone at a dose of 10 mg once per day was substituted for prednisone. Table 10 below summarizes the dosing regimens in cycle I for the two patient cohorts.

TABLE 10
Summary of Cycle 1 Dosing Regimens for AMG
160 and Abiraterone Combination Therapy

| | | Study day¹ | | | |
|---|---|---|---|---|---|
| Cohort | Drug | D 1-D 3 | D 8 | D 15 | D 22 |
| Cohort 1 | AMG 160 | 0.09 mg | 0.15 mg | — | 0.15 mg |
| | | (72 hr eIV) | sIV | | sIV |
| | Abiraterone | — | | 1,000 mg QD | |
| | | | | (C 1 D 15+)² | |
| Cohort 2 & | AMG 160 | 0.09 mg | 0.3 mg | — | 0.3 mg |
| Expansion | | (72 hr eIV) | sIV | | sIV |

| | | | |
|---|---|---|---|
| Cohort | Abiraterone | — | 1,000 mg QD |
| | | | (C 1 D 15+)² |

¹Measured from day 1 (D 1), which was the first day the patient received the first dose of AMG 160 eIV = extended IV infusion; sIV = short IV infusion (approx.. 60 minutes)
²Abiraterone dosing was initiated on cycle 1 day 15 (C 1 D 15) ± 3 days (i.e. between C 1 D 12 and C 1 D 18) and continued QD on each day of the cycle.

All patients were pre-treated with 8 mg PO dexamethasone (or equivalent dose of other corticosteroids) 6-16 hours prior to all doses of AMG 160 in cycle 1. Additionally, dexamethasone 8 mg IV (or equivalent dose of other corticosteroids) was administered within 1 hour prior to all doses of AMG 160 in cycle 1. On cycle 2 day 1, 8 mg dexamethasone IV premedication was given if subjects experienced grade 2 or higher CRS in cycle 1. Otherwise, dexamethasone premedication was reduced in cycle 2, if tolerated (e.g., 4-8 mg dexamethasone on cycle 2 day I and 0-4 mg on cycle 2 day 15). Corticosteroid ophthalmic eye drops (e.g., 1% prednisolone acetate, 2 drops per eye 4 times per day) was used for AMG 160 infusions: (1) within 6 hours prior to start of infusion, (2) during entire infusion period, and (3) for 48 hours after end of infusion in cycles 1 and 2. In cycles 3 and beyond, eye drop prophylaxis was per investigator discretion. Patients received treatment cycles of AMG 160 in combination with abiraterone until disease progression or unacceptable toxicities. Adverse events and anti-tumor efficacy were assessed as described in Example 1.

Four patients were enrolled in cohort 1 and three patients were initially enrolled in cohort 2. At the time of data analysis, all four patients in cohort I continued therapy and two out of the three patients in cohort 2 continued therapy with one patient discontinuing due to radiographic disease progression. All four patients in cohort 1 were DLT evaluable, but no DLTs were observed. Two patients experienced one serious adverse event each, but both events were unrelated to AMG 160 investigational product. Two out of four of the patients experienced grade 2 CRS and the other two patients experienced grade 1 CRS as worst grade. No hepatotoxicity was observed. In terms of preliminary anti-tumor efficacy, all four patients in cohort I had post-baseline PSA measurements and 1 patient had a PSA90 response (confirmed), 1 patient had a PSA70 response (confirmed), and 1 patient had a PSA30 response (confirmed). Two of the four patients, one of which had RECIST measurable target lesions, had on-treatment scans at the time of data analysis with one patient exhibiting a confirmed partial response and the other patient having stable disease.

In cohort 2, two of the three patients were DLT evaluable, one of which developed a DLT (grade 3 rash) on cycle 1 day 9 prior to receiving abiraterone. Thus, the DLT did not occur during combination therapy. There were six adverse events related to the AMG 160 investigational product, but no hepatotoxicity was observed. Two patients had grade 2 CRS and 1 patient bad grade 1 CRS as worst grade. Of the three patients in cohort 2, 1 patient had a PSA70 response (confirmed) and 1 patient had a PSA30 response (confirmed). All three of the patients in cohort 2 had been previously treated with enzalutamide. One patient had also received prior treatment with darolutamide. All three patients in cohort 2 did not have measurable target lesions per RECIST (i.e. were not RECIST evaluable). The results of on-treatment scans for these patients revealed that 2 patients had stable disease and the other patient had unconfirmed progressive disease. Three additional patients are to be enrolled in cohort 2 to further study the combination therapy at this dose level.

An additional three patients were enrolled in cohort 2 and four patients were enrolled in an expansion cohort that received the same dosing regimen as those in cohort 2 (Table 10). A second interim analysis was conducted and a summary of the results from this analysis is provided in Table 11 below. Of the 10 patients enrolled in cohort 2 and the expansion cohort, only two patients had DLTs, only one patient discontinued AMG 160 therapy due to a treatment emergent adverse event, and only one patient had a grade 3 CRS event, which occurred on day 15 of cycle 2. Among the 10 patients enrolled in cohort 2 and the expansion cohort, four had at least a PSA50 response with 2 patients having a PSA90 response. Only 2 of the 10 patients had not received prior therapy with an NHT (i.e. were NHT naïve). Only 2 patients enrolled in cohort 2 and the expansion cohort were RECIST 1.1 evaluable (i.e. having measurable baseline disease as per RECIST 1.1 criteria), one of which had a partial response and the other had stable disease.

The results of this study show that the combination of AMG 160 and abiraterone when abiraterone treatment is initiated about two weeks following initiation of AMG 160 treatment (and about 7 days following administration of the first therapeutic dose of AMG 160) is tolerable and leads to reductions in PSA serum levels in the majority of patients and an objective response rate of 50%.

TABLE 11
Summary of Interim Results of AMG 160 and
Abiraterone Combination Therapy for mCRPC

AMG 160 therapeutic dose

| | | |
|---|---|---|
| 0.15 mg | 0.30 mg | 0.30 mg |

Start of abiraterone

| | C 1 D 15 | C 1 D 15 | C 1 D 15 |
|---|---|---|---|
| Cohort | Cohort 1 | Cohort 2 | Expansion |
| Number of subjects | 4 | 6 | 4 |
| TEAEs (any grade)^{a} | 4 (100%) | 6 (100%) | 4 (100%) |
| TEAEs (grade ≥3)^{a} | 3 (75%) | 4 (66.7%) | 2 (50%) |
| SAEs^{a} | 2 (50%) | 4 (66.7%) | 3 (75%) |
| TEAEs leading to | 0 (0%) | 1 (16.7%) | 0 (0%) |
| discontinuation of AMG | | | |
| 160^{a} | | | |
| Number of subjects with | 0 (0%) | 2 (40%)^{b} | 0 (0%) |
| DLT | | | |
| CRS (any grade)^{a} | 4 (100%) | 6 (100%) | 4 (100%) |
| CRS (grade ≥3)^{a} | 0 (0%) | 0 (0%) | 1 (25%) |
| Concomitant medication | | | |
| use for CRS treatment:^{a} | | | |
| Oxygen | 0 (0%) | 2 (33.3%) | 0 (0%) |
| Norepinephrine | 0 (0%) | 1 (16.7%) | 0 (0%) |
| Dexamethasone | 1 (25%) | 1 (16.7%) | 0 (0%) |
| Tocilizumab | 0 (0%) | 2 (33.3%) | 0 (0%) |
| PSA50 response^{a} | 2 (50%) | 3 (50%) | 1 (25%) |
| PSA90 response^{a} | 1 (25%) | 1 (16.7%) | 1 (25%) |
| RECIST 1.1 evaluable | 2 | 1 | 1 |
| ORR (%) | 1 (50%) | 1 (100%) | 0 (0%) |

^{a}Subject incidence for each of the indicated events
^{b}5 of 6 subjects in cohort 2 were DLT evaluable. One DLT event (grade 3 rash) occurred on C 1 D 9 prior to receiving abiraterone (i.e. outside combination therapy window)

All publications, patents, and patent applications discussed and cited herein are hereby incorporated by reference in their entireties. It is understood that the disclosed invention is not limited to the particular methodology, protocols and materials described as these can vary. It is also understood that the terminology used herein is for the purposes of describing particular embodiments only and is not intended to limit the scope of the appended claims.

Those skilled in the art will recognize, or be able to ascertain using no more than routine experimentation, many equivalents to the specific embodiments of the invention described herein. Such equivalents are intended to be encompassed by the following claims.

1. A method for treating prostate cancer in a patient in need thereof, comprising administering to the patient one or more cycles of an anti-androgen compound in combination with a T-cell engaging molecule that specifically binds to human prostate-specific membrane antigen (PSMA) and human CD3, wherein a first cycle comprises administering a first dose of the anti-androgen compound at least 3 days after administering a first therapeutic dose of the T-cell engaging molecule.

2. The method of claim 1, wherein the first cycle comprises administering the first dose of the anti-androgen compound about 4 days to about 10 days after administering the first therapeutic dose of the T-cell engaging molecule.

3. The method of claim 1, wherein the first cycle comprises administering the first dose of the anti-androgen compound about 5 days after administering the first therapeutic dose of the T-cell engaging molecule.

4. The method of claim 1, wherein the first cycle comprises administering the first dose of the anti-androgen compound about 7 days after administering the first therapeutic dose of the T-cell engaging molecule.

5. The method of claim 1, wherein the T-cell engaging molecule is administered by intravenous infusion.

6. The method of claim 1, wherein the first cycle further comprises administering one or more priming doses of the T-cell engaging molecule prior to administering the first therapeutic dose of the T-cell engaging molecule.

7. The method of claim 6, wherein the first priming dose of the T-cell engaging molecule is administered by continuous intravenous infusion over a period of 1 day to 7 days.

8. The method of claim 6, wherein the first therapeutic dose of the T-cell engaging molecule is administered about 1 day to about 7 days after administration of the first priming dose of the T-cell engaging molecule.

9. The method of claim 8, wherein the first therapeutic dose of the T-cell engaging molecule is administered about 5 days after administration of the first priming dose of the T-cell engaging molecule.

10. The method of claim 1, wherein the anti-androgen compound is administered orally once per day.

11. The method of claim 1, wherein the duration of the first cycle is about 28 days.

12. The method of claim 11, wherein the first cycle comprises administering a priming dose of the T-cell engaging molecule by continuous intravenous infusion over days 1 to 3 of the cycle and administering a therapeutic dose of the T-cell engaging molecule by a bolus intravenous infusion on days 8 and 22 of the cycle.

13. The method of claim 11, wherein the T-cell engaging molecule is administered on days 1, 8, 15, and 22 of the first cycle.

14. The method of claim 11, wherein the T-cell engaging molecule is administered on days 1 and 15 of the first cycle.

15. The method of claim 11, wherein the anti-androgen compound is administered orally once per day on each of days 15 to 28 of the first cycle.

16. The method of claim 1, wherein the anti-androgen compound is enzalutamide, abiraterone, abiraterone acetate, apalutamide, or darolutamide.

17. The method of claim 16, wherein the anti-androgen compound is enzalutamide.

18. The method of claim 17, wherein enzalutamide is administered orally at a dose of about 160 mg once per day.

19. The method of claim 16, wherein the anti-androgen compound is abiraterone or abiraterone acetate.

20. The method of claim 19, wherein abiraterone or abiraterone acetate is administered orally at a dose of about 1,000 mg once per day.

21. The method of claim 1, wherein the T-cell engaging molecule comprises, in an amino to carboxyl order:
(i) a first domain that specifically binds to human PSMA comprising a first immunoglobulin heavy chain variable region (VH1) and a first immunoglobulin light chain variable region (VL1);
(ii) a second domain that specifically binds to human CD3 comprising a second immunoglobulin heavy chain variable region (VH2), and a second immunoglobulin light chain variable region (VL2); and
(iii) an Fc domain comprising two Fc monomers, each monomer comprising an immunoglobulin hinge region, a CH2 domain, and a CH3 domain, wherein said two Fc monomers are fused to each other via a peptide linker.

22. The method of claim 21, wherein the first domain comprises a VH1 comprising a CDRH1 having the sequence of SEQ ID NO: 14, a CDRH2 having the sequence of SEQ ID NO: 16, and a CDRH3 having the sequence of SEQ ID NO: 20, and a VL1 comprising a CDRL1 having the sequence of SEQ ID NO: 5, a CDRL2 having the sequence of SEQ ID NO: 8, and a CDRL3 having the sequence of SEQ ID NO: 9; and
wherein the second domain comprises a VH2 comprising a CDRH1 having the sequence of SEQ ID NO: 49, a CDRH2 having the sequence of SEQ ID NO: 55, and a CDRH3 having the sequence of SEQ ID NO: 60, and a VL2 comprising a CDRL1 having the sequence of SEQ ID NO: 43, a CDRL2 having the sequence of SEQ ID NO: 44, and a CDRL3 having the sequence of SEQ ID NO: 47.

23. The method of claim 22, wherein VH1 comprises the sequence of SEQ ID NO: 33, VL1 comprises the sequence of SEQ ID NO: 30, VH2 comprises the sequence of SEQ ID NO: 72, and VL2 comprises the sequence of SEQ ID NO: 70.

24. The method of claim 21, wherein the T-cell engaging molecule is a single chain polypeptide comprising the sequence of SEQ ID NO: 140.

25. The method of claim 21, wherein the first cycle is about 28 days and comprises:
administering a priming dose of about 90 μg of the T-cell engaging molecule by continuous intravenous infusion over days 1 to 3 of the cycle;
administering a therapeutic dose of about 300 μg of the T-cell engaging molecule by a bolus intravenous infusion on days 8 and 22 of the cycle; and
administering an anti-androgen compound orally once per day on each of days 15 to 28 of the cycle.

26. The method of claim 21, wherein the first cycle is about 28 days and comprises:
administering a priming dose of about 90 μg of the T-cell engaging molecule by continuous intravenous infusion over days 1 to 3 of the cycle;
administering a therapeutic dose of about 150 μg of the T-cell engaging molecule by a bolus intravenous infusion on days 8 and 22 of the cycle; and
administering an anti-androgen compound orally once per day on each of days 15 to 28 of the cycle.

27. The method of claim 1, further comprising administering to the patient a maintenance cycle of the anti-androgen compound in combination with a T-cell engaging molecule, wherein the maintenance cycle comprises administering the anti-androgen compound orally once per day on each day of the cycle and administering a therapeutic dose of the T-cell engaging molecule by a bolus intravenous infusion once every 7 days or once every 14 days.

28. The method of claim 27, wherein the duration of the maintenance cycle is about 28 days.

29. The method of claim 27, wherein the maintenance cycle is administered about 7 days following completion of the first cycle.

30. The method of claim 27, wherein two or more maintenance cycles are administered to the patient.

31. The method of claim 1, wherein the prostate cancer is metastatic prostate cancer.

32. The method of claim 31, wherein the prostate cancer is metastatic castration-resistant prostate cancer.

33. The method of claim 1, wherein the patient has total serum testosterone levels of 50 ng/dL or less.

34-67. (canceled)


SEQUENCE LISTING (192 sequences)

SEQ ID NO: 1 - protein, 750 aa, Homo sapiens
MWNLLHETDSAVATARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 2 - protein, 750 aa, Macaca mulatta
MWNLLHETDSAVATARRPRWLCAGALVLAGGFFLLGFLFGWFIKSSSEATNITPKHNMKAFLDELKAENIKKFLHNFTQIPHLAGTEQNFQLAKQIQSQWKEFGLDSVELTHYDVLLSYPNKTHPNYISIINEDGNEIFNTSLFEPPPAGYENVSDIVPPFSAFSPQGMPEGDLVYVNYARTEDFFKLERDMKINCSGKIVIARYGKVFRGNKVKNAQLAGATGVILYSDPADYFAPGVKSYPDGWNLPGGGVQRGNILNLNGAGDPLTPGYPANEYAYRRGMAEAVGLPSIPVHPIGYYDAQKLLEKMGGSASPDSSWRGSLKVPYNVGPGFTGNFSTQKVKMHIHSTSEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVYNLTKELESPDEGFEGKSLYESWTKKSPSPEFSGMPRISKLGSGNDFEVFFQRLGIASGRARYTKNWETNKFSSYPLYHSVYETYELVEKFYDPMFKYHLTVAQVRGGMVFELANSVVLPFDCRDYAVVLRKYADKIYNISMKHPQEMKTYSVSFDSLFSAVKNFTEIASKFSERLRDFDKSNPILLRMMNDQLMFLERAFIDPLGLPDRPFYRHVIYAPSSHNKYAGESFPGIYDALFDIESKVDPSQAWGEVKRQISVATFTVQAAAETLSEVA

SEQ ID NO: 3 - protein, 707 aa, Homo sapiens
KSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 4 - protein, 707 aa, Macaca mulatta
KSSSEATNITPKHNMKAFLDELKAENIKKFLHNFTQIPHLAGTEQNFQLAKQIQSQWKEFGLDSVELTHYDVLLSYPNKTHPNYISIINEDGNEIFNTSLFEPPPAGYENVSDIVPPFSAFSPQGMPEGDLVYVNYARTEDFFKLERDMKINCSGKIVIARYGKVFRGNKVKNAQLAGATGVILYSDPADYFAPGVKSYPDGWNLPGGGVQRGNILNLNGAGDPLTPGYPANEYAYRRGMAEAVGLPSIPVHPIGYYDAQKLLEKMGGSASPDSSWRGSLKVPYNVGPGFTGNFSTQKVKMHIHSTSEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVYNLTKELESPDEGFEGKSLYESWTKKSPSPEFSGMPRISKLGSGNDFEVFFQRLGIASGRARYTKNWETNKFSSYPLYHSVYETYELVEKFYDPMFKYHLTVAQVRGGMVFELANSVVLPFDCRDYAVVLRKYADKIYNISMKHPQEMKTYSVSFDSLFSAVKNFTEIASKFSERLRDFDKSNPILLRMMNDQLMFLERAFIDPLGLPDRPFYRHVIYAPSSHNKYAGESFPGIYDALFDIESKVDPSQAWGEVKRQISVATFTVQAAAETLSEVA

SEQ ID NO: 5 - protein, 11 aa, synthetic construct
KASQNVDTNVA

SEQ ID NO: 6 - protein, 11 aa, synthetic construct
KASQNVDANVA

SEQ ID NO: 7 - protein, 7 aa, synthetic construct
SASYRYS

SEQ ID NO: 8 - protein, 7 aa, synthetic construct
SASYVYW

SEQ ID NO: 9 - protein, 9 aa, synthetic construct
QQYDQQLIT

SEQ ID NO: 10 - protein, 9 aa, synthetic construct
QQYDQVRLT

SEQ ID NO: 11 - protein, 9 aa, synthetic construct
QQYDSYPYT

SEQ ID NO: 12 - protein, 9 aa, synthetic construct
QQYDRQPLT

SEQ ID NO: 13 - protein, 9 aa, synthetic construct
QQYDAPMGT

SEQ ID NO: 14 - protein, 5 aa, synthetic construct
DYYMY

SEQ ID NO: 15 - protein, 5 aa, synthetic construct
DYYMN

SEQ ID NO: 16 - protein, 17 aa, synthetic construct
IISDGGYYTYYSDIIKG

SEQ ID NO: 17 - protein, 17 aa, synthetic construct
IISDHHHWTYYSDIIKG

SEQ ID NO: 18 - protein, 17 aa, synthetic construct
IISDNKHQTYYSDIIKG

SEQ ID NO: 19 - protein, 17 aa, synthetic construct
IISDAGYYTYYSDIIKG

SEQ ID NO: 20 - protein, 12 aa, synthetic construct
GFPLLRHGAMDY

SEQ ID NO: 21 - protein, 107 aa, synthetic construct
DIQMTQSPSSLSASVGDRVTITCKASQNVDTNVAWYQQKPGQAPKSLIYSASYRYSDVPSRFSGSASGTDFTLTISSVQSEDFATYYCQQYDQQLITFGCGTKLEIK

SEQ ID NO: 22 - protein, 107 aa, synthetic construct
DIQMTQSPSSLSASVGDRVTITCKASQNVDTNVAWYQQKPGQAPKSLIYSASYRYSDVPSRFSGSASGTDFTLTISSVQSEDFATYYCQQYDQVRLTFGCGTKLEIK

SEQ ID NO: 23 - protein, 107 aa, synthetic construct
DIQMTQSPSSLSASVGDRVTITCKASQNVDTNVAWYQQKPGQAPKSLIYSASYRYSDVPSRFSGSASGTDFTLTISSVQSEDFATYYCQQYDSYPYTFGCGTKLEIK

SEQ ID NO: 24 - protein, 107 aa, synthetic construct
DIQMTQSPSSLSASVGDRVTITCKASQNVDTNVAWYQQKPGQAPKSLIYSASYRYSDVPSRFSGSASGTDFTLTISSVQSEDFATYYCQQYDRQPLTFGCGTKLEIK

SEQ ID NO: 25 - protein, 107 aa, synthetic construct
DIQMTQSPSSLSASVGDRVTITCKASQNVDTNVAWYQQKPGQAPKSLIYSASYRYSDVPSRFSGSASGTDFTLTISSVQSEDFATYYCQQYDAPMGTFGCGTKLEIK

SEQ ID NO: 26 - protein, 107 aa, synthetic construct
DIQMTQSPSSLSASVGDRVTITCKASQNVDTNVAWYQQKPGQAPKSLIYSASYRYSDVPSRFSGSASGTDFTLTISSLQSEDFATYYCQQYDSYPYTFGGGTKLEIK

SEQ ID NO: 27 - protein, 107 aa, synthetic construct
DIQMTQSPSSLSASVGDRVTITCKASQNVDTNVAWYQQKPGQAPKSLIYSASYRYSDVPSRFSGSASGTDFTLTISSLQSEDFATYYCQQYDSYPYTFGCGTKLEIK

SEQ ID NO: 28 - protein, 107 aa, synthetic construct
DIQMTQSPSSLSASVGDRVTITCKASQNVDTNVAWYQQKPGQAPKSLIYSASYRYSDVPSRFSGSASGTDFTLTISSVQSEDFATYYCQQYDSYPYTFGGGTKLEIK

SEQ ID NO: 29 - protein, 107 aa, synthetic construct
DIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGGGTKLEIK

SEQ ID NO: 30 - protein, 107 aa, synthetic construct
DIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGCGTKLEIK

SEQ ID NO: 31 - protein, 107 aa, synthetic construct
DIQMTQSPSSLSASVGDRVTITCKASQNVDANVAWYQQKPGQAPKSLIYSASYVYWDVPSRFSGSASGTDFTLTISSVQSEDFATYYCQQYDQQLITFGGGTKLEIK

SEQ ID NO: 32 - protein, 107 aa, synthetic construct
DIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIK

SEQ ID NO: 33 - protein, 121 aa, synthetic construct
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSS

SEQ ID NO: 34 - protein, 121 aa, synthetic construct
QVQLVESGGGLVKPGESLRLSCAASGFTFSDYYMYWVRQAPGKCLEWVAIISDHHHWTYYSDIIKGRFTISRDNAKNSLYLQMNSLKAEDTAVYYCARGFPLLRHGAMDYWGQGTLVTVSS

SEQ ID NO: 35 - protein, 121 aa, synthetic construct
QVQLVESGGGLVKPGESLRLSCAASGFTFSDYYMNWVRQAPGKGLEWVAIISDNKHQTYYSDIIKGRFAISRDNAKNSLYLQMNSLKAEDTAVYYCARGFPLLRHGAMDYWGQGTLVTVSS

SEQ ID NO: 36 - protein, 121 aa, synthetic construct
QVQLVESGGGLVKPGESLRLSCAASGFTFSDYYMNWVRQAPGKCLEWVAIISDNKHQTYYSDIIKGRFAISRDNAKNSLYLQMNSLKAEDTAVYYCARGFPLLRHGAMDYWGQGTLVTVSS

SEQ ID NO: 37 - protein, 121 aa, synthetic construct
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSS

SEQ ID NO: 38 - protein, 121 aa, synthetic construct
QVQLVESGGGLVKPGESLRLSCAASGFTFSDYYMYWVRQAPGKGLEWVAIISDAGYYTYYSDIIKGRFTISRDNAKNSLYLQMNSLKAEDTAVYYCARGFPLLRHGAMDYWGQGTLVTVSS

SEQ ID NO: 39 - protein, 121 aa, synthetic construct
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSS

SEQ ID NO: 40 - protein, 105 aa, Homo sapiens
QDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMD

SEQ ID NO: 41 - protein, 14 aa, synthetic construct
GSSTGAVTSGYYPN

SEQ ID NO: 42 - protein, 14 aa, synthetic construct
RSSTGAVTSGYYPN

SEQ ID NO: 43 - protein, 14 aa, synthetic construct
GSSTGAVTSGNYPN

SEQ ID NO: 44 - protein, 7 aa, synthetic construct
GTKFLAP

SEQ ID NO: 45 - protein, 7 aa, synthetic construct
ATDMRPS

SEQ ID NO: 46 - protein, 9 aa, synthetic construct
ALWYSNRWV

SEQ ID NO: 47 - protein, 9 aa, synthetic construct
VLWYSNRWV

SEQ ID NO: 48 - protein, 5 aa, synthetic construct
IYAMN

SEQ ID NO: 49 - protein, 5 aa, synthetic construct
KYAMN

SEQ ID NO: 50 - protein, 5 aa, synthetic construct
SYAMN

SEQ ID NO: 51 - protein, 5 aa, synthetic construct
RYAMN

SEQ ID NO: 52 - protein, 5 aa, synthetic construct
VYAMN

SEQ ID NO: 53 - protein, 5 aa, synthetic construct
GYAMN

SEQ ID NO: 54 - protein, 19 aa, synthetic construct
RIRSKYNNYATYYADSVKS

SEQ ID NO: 55 - protein, 19 aa, synthetic construct
RIRSKYNNYATYYADSVKD

SEQ ID NO: 56 - protein, 19 aa, synthetic construct
RIRSKYNNYATYYADSVKG

SEQ ID NO: 57 - protein, 19 aa, synthetic construct
RIRSKYNNYATYYADSVKK

SEQ ID NO: 58 - protein, 19 aa, synthetic construct
RIRSKYNNYATYYADSVKE

SEQ ID NO: 59 - protein, 14 aa, synthetic construct
HGNFGNSYVSFFAY

SEQ ID NO: 60 - protein, 14 aa, synthetic construct
HGNFGNSYISYWAY

SEQ ID NO: 61 - protein, 14 aa, synthetic construct
HGNFGNSYLSFWAY

SEQ ID NO: 62 - protein, 14 aa, synthetic construct
HGNFGNSYLSYFAY

SEQ ID NO: 63 - protein, 14 aa, synthetic construct
HGNFGNSYLSWWAY

SEQ ID NO: 64 - protein, 14 aa, synthetic construct
HGNFGNSYTSYYAY

SEQ ID NO: 65 - protein, 14 aa, synthetic construct
HRNFGNSYLSWFAY

SEQ ID NO: 66 - protein, 14 aa, synthetic construct
HGNFGNSYISWWAY

SEQ ID NO: 67 - protein, 14 aa, synthetic construct
HGNFGNSYVSWWAY

SEQ ID NO: 68 - protein, 109 aa, synthetic construct
QTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 69 - protein, 109 aa, synthetic construct
QTVVTQEPSLTVSPGGTVTLTCRSSTGAVTSGYYPNWVQQKPGQAPRGLIGATDMRPSGTPARFSGSLLGGKAALTLSGVQPEDEAEYYCALWYSNRWVFGGGTKLTVL

SEQ ID NO: 70 - protein, 109 aa, synthetic construct
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 71 - protein, 125 aa, synthetic construct
EVQLVESGGGLVQPGGSLKLSCAASGFTFNIYAMNWVRQAPGKGLEWVARIRSKYNNYATYYADSVKSRFTISRDDSKNTAYLQMNNLKTEDTAVYYCVRHGNFGNSYVSFFAYWGQGTLVTVSS

SEQ ID NO: 72 - protein, 125 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSS

SEQ ID NO: 73 - protein, 125 aa, synthetic construct
EVQLVESGGGLEQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYLSFWAYWGQGTLVTVSS

SEQ ID NO: 74 - protein, 125 aa, synthetic construct
EVQLVESGGGLVQPGGSLKLSCAASGFTFNRYAMNWVRQAPGKGLEWVARIRSKYNNYATYYADSVKGRFTISRDDSKNTAYLQMNNLKTEDTAVYYCVRHGNFGNSYLSYFAYWGQGTLVTVSS

SEQ ID NO: 75 - protein, 125 aa, synthetic construct
EVQLVESGGGLVQPGGSLKLSCAASGFTFNVYAMNWVRQAPGKGLEWVARIRSKYNNYATYYADSVKKRFTISRDDSKNTAYLQMNNLKTEDTAVYYCVRHGNFGNSYLSWWAYWGQGTLVTVSS

SEQ ID NO: 76 - protein, 125 aa, synthetic construct
EVQLVESGGGLVQPGGSLKLSCAASGFTFNKYAMNWVRQAPGKGLEWVARIRSKYNNYATYYADSVKSRFTISRDDSKNTAYLQMNNLKTEDTAVYYCVRHGNFGNSYTSYYAYWGQGTLVTVSS

SEQ ID NO: 77 - protein, 125 aa, synthetic construct
EVQLVESGGGLVQPGGSLKLSCAASGFTFNGYAMNWVRQAPGKGLEWVARIRSKYNNYATYYADSVKERFTISRDDSKNTAYLQMNNLKTEDTAVYYCVRHRNFGNSYLSWFAYWGQGTLVTVSS

SEQ ID NO: 78 - protein, 125 aa, synthetic construct
EVQLVESGGGLVQPGGSLKLSCAASGFTFNVYAMNWVRQAPGKGLEWVARIRSKYNNYATYYADSVKKRFTISRDDSKNTAYLQMNNLKTEDTAVYYCVRHGNFGNSYISWWAYWGQGTLVTVSS

SEQ ID NO: 79 - protein, 125 aa, synthetic construct
EVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSS

SEQ ID NO: 80 - protein, 4 aa, synthetic construct
GGGG

SEQ ID NO: 81 - protein, 5 aa, synthetic construct
GGGGS

SEQ ID NO: 82 - protein, 10 aa, synthetic construct
GGGGSGGGGS

SEQ ID NO: 83 - protein, 15 aa, synthetic construct
GGGGSGGGGSGGGGS

SEQ ID NO: 84 - protein, 20 aa, synthetic construct
GGGGSGGGGSGGGGSGGGGS

SEQ ID NO: 85 - protein, 25 aa, synthetic construct
GGGGSGGGGSGGGGSGGGGSGGGGS

SEQ ID NO: 86 - protein, 30 aa, synthetic construct
GGGGSGGGGSGGGGSGGGGSGGGGSGGGGS

SEQ ID NO: 87 - protein, 35 aa, synthetic construct
GGGGSGGGGSGGGGSGGGGSGGGGSGGGGSGGGGS

SEQ ID NO: 88 - protein, 40 aa, synthetic construct
GGGGSGGGGSGGGGSGGGGSGGGGSGGGGSGGGGSGGGGS

SEQ ID NO: 89 - protein, 6 aa, synthetic construct
PGGGGS

SEQ ID NO: 90 - protein, 6 aa, synthetic construct
PGGDGS

SEQ ID NO: 91 - protein, 6 aa, synthetic construct
SGGGGS

SEQ ID NO: 92 - protein, 9 aa, synthetic construct
GGGGSGGGS

SEQ ID NO: 93 - protein, 5 aa, synthetic construct
GGGGQ

SEQ ID NO: 94 - protein, 243 aa, synthetic construct
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYRYSDVPSRFSGSASGTDFTLTISSVQSEDFATYYCQQYDQQLITFGCGTKLEIK

SEQ ID NO: 95 - protein, 243 aa, synthetic construct
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYRYSDVPSRFSGSASGTDFTLTISSVQSEDFATYYCQQYDQVRLTFGCGTKLEIK

SEQ ID NO: 96 - protein, 243 aa, synthetic construct
QVQLVESGGGLVKPGESLRLSCAASGFTFSDYYMYWVRQAPGKCLEWVAIISDHHHWTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYRYSDVPSRFSGSASGTDFTLTISSVQSEDFATYYCQQYDSYPYTFGCGTKLEIK

SEQ ID NO: 97 - protein, 243 aa, synthetic construct
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYRYSDVPSRFSGSASGTDFTLTISSVQSEDFATYYCQQYDRQPLTFGCGTKLEIK

SEQ ID NO: 98 - protein, 243 aa, synthetic construct
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYRYSDVPSRFSGSASGTDFTLTISSVQSEDFATYYCQQYDAPMGTFGCGTKLEIK

SEQ ID NO: 99 - protein, 243 aa, synthetic construct
QVQLVESGGGLVKPGESLRLSCAASGFTFSDYYMNWVRQAPGKGLEWVAIISDNKHQTYYSDIIKGRFAISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYRYSDVPSRFSGSASGTDFTLTISSLQSEDFATYYCQQYDSYPYTFGGGTKLEIK

SEQ ID NO: 100 - protein, 243 aa, synthetic construct
QVQLVESGGGLVKPGESLRLSCAASGFTFSDYYMNWVRQAPGKCLEWVAIISDNKHQTYYSDIIKGRFAISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYRYSDVPSRFSGSASGTDFTLTISSLQSEDFATYYCQQYDSYPYTFGCGTKLEIK

SEQ ID NO: 101 - protein, 243 aa, synthetic construct
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYRYSDVPSRFSGSASGTDFTLTISSVQSEDFATYYCQQYDSYPYTFGGGTKLEIK

SEQ ID NO: 102 - protein, 243 aa, synthetic construct
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYRYSDVPSRFSGSASGTDFTLTISSVQSEDFATYYCQQYDSYPYTFGCGTKLEIK

SEQ ID NO: 103 - protein, 243 aa, synthetic construct
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGGGTKLEIK

SEQ ID NO: 104 - protein, 243 aa, synthetic construct
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGCGTKLEIK

SEQ ID NO: 105 - protein, 243 aa, synthetic construct
QVQLVESGGGLVKPGESLRLSCAASGFTFSDYYMYWVRQAPGKGLEWVAIISDAGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDANVAWYQQKPGQAPKSLIYSASYVYWDVPSRFSGSASGTDFTLTISSVQSEDFATYYCQQYDQQLITFGGGTKLEIK

SEQ ID NO: 106 - protein, 243 aa, synthetic construct
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIK

SEQ ID NO: 107 - protein, 249 aa, synthetic construct
EVQLVESGGGLVQPGGSLKLSCAASGFTFNIYAMNWVRQAPGKGLEWVARIRSKYNNYATYYADSVKSRFTISRDDSKNTAYLQMNNLKTEDTAVYYCVRHGNFGNSYVSFFAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 108 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 109 - protein, 249 aa, synthetic construct
EVQLVESGGGLEQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYLSFWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 110 - protein, 249 aa, synthetic construct
EVQLVESGGGLVQPGGSLKLSCAASGFTFNRYAMNWVRQAPGKGLEWVARIRSKYNNYATYYADSVKGRFTISRDDSKNTAYLQMNNLKTEDTAVYYCVRHGNFGNSYLSYFAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 111 - protein, 249 aa, synthetic construct
EVQLVESGGGLVQPGGSLKLSCAASGFTFNVYAMNWVRQAPGKGLEWVARIRSKYNNYATYYADSVKKRFTISRDDSKNTAYLQMNNLKTEDTAVYYCVRHGNFGNSYLSWWAYWGQGTLVTVSSGGGGSGGGGSGGGGSQTVVTQEPSLTVSPGGTVTLTCRSSTGAVTSGYYPNWVQQKPGQAPRGLIGATDMRPSGTPARFSGSLLGGKAALTLSGVQPEDEAEYYCALWYSNRWVFGGGTKLTVL

SEQ ID NO: 112 - protein, 249 aa, synthetic construct
EVQLVESGGGLVQPGGSLKLSCAASGFTFNKYAMNWVRQAPGKGLEWVARIRSKYNNYATYYADSVKSRFTISRDDSKNTAYLQMNNLKTEDTAVYYCVRHGNFGNSYTSYYAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 113 - protein, 249 aa, synthetic construct
EVQLVESGGGLVQPGGSLKLSCAASGFTFNGYAMNWVRQAPGKGLEWVARIRSKYNNYATYYADSVKERFTISRDDSKNTAYLQMNNLKTEDTAVYYCVRHRNFGNSYLSWFAYWGQGTLVTVSSGGGGSGGGGSGGGGSQTVVTQEPSLTVSPGGTVTLTCRSSTGAVTSGYYPNWVQQKPGQAPRGLIGATDMRPSGTPARFSGSLLGGKAALTLSGVQPEDEAEYYCALWYSNRWVFGGGTKLTVL

SEQ ID NO: 114 - protein, 249 aa, synthetic construct
EVQLVESGGGLVQPGGSLKLSCAASGFTFNVYAMNWVRQAPGKGLEWVARIRSKYNNYATYYADSVKKRFTISRDDSKNTAYLQMNNLKTEDTAVYYCVRHGNFGNSYISWWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVL

SEQ ID NO: 115 - protein, 249 aa, synthetic construct
EVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 116 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 117 - protein, 10 aa, synthetic construct
DKTHTCPPCP

SEQ ID NO: 118 - protein, 15 aa, synthetic construct
EPKSCDKTHTCPPCP

SEQ ID NO: 119 - protein, 12 aa, synthetic construct
ERKCCVECPPCP

SEQ ID NO: 120 - protein, 16 aa, synthetic construct
ELKTPLDTTHTCPRCP

SEQ ID NO: 121 - protein, 15 aa, synthetic construct
EPKSCDTPPPCPRCP

SEQ ID NO: 122 - protein, 17 aa, synthetic construct
ELKTPLGDTTHTCPRCP

SEQ ID NO: 123 - protein, 12 aa, synthetic construct
ESKYGPPCPSCP

SEQ ID NO: 124 - protein, 227 aa, synthetic construct
DKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 125 - protein, 225 aa, synthetic construct
DKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 126 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 127 - protein, 225 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 128 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 129 - protein, 225 aa, synthetic construct
DKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 130 - protein, 227 aa, synthetic construct
DKTHTCPPCPAPELLGGPSVFLFPPKPKDTLMISRTPEVTCVVVDVSHEDPEVKFNWYVDGVEVHNAKTKPCEEQYNSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 131 - protein, 225 aa, synthetic construct
DKTHTCPPCPAPELLGGPSVFLFPPKPKDTLMISRTPEVTCVVVDVSHEDPEVKFNWYVDGVEVHNAKTKPCEEQYNSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 132 - protein, 484 aa, synthetic construct
DKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 133 - protein, 480 aa, synthetic construct
DKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 134 - protein, 484 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 135 - protein, 480 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 136 - protein, 484 aa, synthetic construct
DKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 137 - protein, 480 aa, synthetic construct
DKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 138 - protein, 484 aa, synthetic construct
DKTHTCPPCPAPELLGGPSVFLFPPKPKDTLMISRTPEVTCVVVDVSHEDPEVKFNWYVDGVEVHNAKTKPCEEQYNSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYNSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 139 - protein, 480 aa, synthetic construct
DKTHTCPPCPAPELLGGPSVFLFPPKPKDTLMISRTPEVTCVVVDVSHEDPEVKFNWYVDGVEVHNAKTKPCEEQYNSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYNSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 140 - protein, 986 aa, synthetic construct
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 141 - protein, 982 aa, synthetic construct
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 142 - protein, 986 aa, synthetic construct
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYRYSDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 143 - protein, 986 aa, synthetic construct
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYRYSDVPSRFSGSASGTDFTLTISSVQSEDFATYYCQQYDQVRLTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 144 - protein, 986 aa, synthetic construct
QVQLVESGGGLVKPGESLRLSCAASGFTFSDYYMYWVRQAPGKCLEWVAIISDHHHWTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYRYSDVPSRFSGSASGTDFTLTISSVQSEDFATYYCQQYDSYPYTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 145 - protein, 986 aa, synthetic construct
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYRYSDVPSRFSGSASGTDFTLTISSVQSEDFATYYCQQYDRQPLTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 146 - protein, 986 aa, synthetic construct
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYRYSDVPSRFSGSASGTDFTLTISSVQSEDFATYYCQQYDAPMGTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 147 - protein, 986 aa, synthetic construct
QVQLVESGGGLVKPGESLRLSCAASGFTFSDYYMNWVRQAPGKGLEWVAIISDNKHQTYYSDIIKGRFAISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYRYSDVPSRFSGSASGTDFTLTISSLQSEDFATYYCQQYDSYPYTFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 148 - protein, 986 aa, synthetic construct
QVQLVESGGGLVKPGESLRLSCAASGFTFSDYYMNWVRQAPGKCLEWVAIISDNKHQTYYSDIIKGRFAISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYRYSDVPSRFSGSASGTDFTLTISSLQSEDFATYYCQQYDSYPYTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 149 - protein, 986 aa, synthetic construct
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYRYSDVPSRFSGSASGTDFTLTISSVQSEDFATYYCQQYDSYPYTFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 150 - protein, 986 aa, synthetic construct
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYRYSDVPSRFSGSASGTDFTLTISSVQSEDFATYYCQQYDSYPYTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 151 - protein, 986 aa, synthetic construct
QVQLVESGGGLVKPGESLRLSCAASGFTFSDYYMYWVRQAPGKGLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 152 - protein, 986 aa, synthetic construct
QVQLVESGGGLVKPGESLRLSCAASGFTFSDYYMYWVRQAPGKGLEWVAIISDAGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDANVAWYQQKPGQAPKSLIYSASYVYWDVPSRFSGSASGTDFTLTISSVQSEDFATYYCQQYDQQLITFGGGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 153 - protein, 986 aa, synthetic construct
QVQLVESGGGLVKPGESLRLSCAASGFTFSDYYMYWVRQAPGKCLEWVAIISDAGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDANVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 154 - protein, 986 aa, synthetic construct
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 155 - protein, 986 aa, synthetic construct
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYVYWDVPSRFSGSASGTDFTLTISSVQSEDFATYYCQQYDQQLITFGCGTKLEIKSGGGGSEVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 156 - protein, 986 aa, synthetic construct
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYRYSDVPSRFSGSASGTDFTLTISSVQSEDFATYYCQQYDSYPYTFGCGTKLEIKSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGYYPNWVQQKPGQAPRGLIGGTKFLAPGTPARFSGSLLGGKAALTLSGVQPEDEAEYYCALWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 157 - protein, 986 aa, synthetic construct
QVQLVESGGGLVKPGESLRLSCAASGFTFSDYYMYWVRQAPGKCLEWVAIISDGGYYTYYSDIIKGRFTISRDNAKNSLYLQMNSLKAEDTAVYYCARGFPLLRHGAMDYWGQGTLVTVSSGGGGSGGGGSGGGGSDIQMTQSPSSLSASVGDRVTITCKASQNVDTNVAWYQQKPGQAPKSLIYSASYRYSDVPSRFSGSASGTDFTLTISSVQSEDFATYYCQQYDSYPYTFGCGTKLEIKSGGGGSEVQLVESGGGLVQPGGSLKLSCAASGFTFNSYAMNWVRQAPGKGLEWVARIRSKYNNYATYYADSVKGRFTISRDDSKNTAYLQMNNLKTEDTAVYYCVRHGNFGNSYVSWWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 158 - protein, 10 aa, synthetic construct
GGSISSSNYF

SEQ ID NO: 159 - protein, 8 aa, synthetic construct
GFSFSRYG

SEQ ID NO: 160 - protein, 10 aa, synthetic construct
GGSISSSSYY

SEQ ID NO: 161 - protein, 8 aa, synthetic construct
GFSFRSYG

SEQ ID NO: 162 - protein, 8 aa, synthetic construct
GFSFSSYG

SEQ ID NO: 163 - protein, 7 aa, synthetic construct
IDYSGYT

SEQ ID NO: 164 - protein, 8 aa, synthetic construct
IWYDGSNR

SEQ ID NO: 165 - protein, 7 aa, synthetic construct
VDYSGYT

SEQ ID NO: 166 - protein, 8 aa, synthetic construct
IWYDGSNK

SEQ ID NO: 167 - protein, 12 aa, synthetic construct
ARHKAATADFDY

SEQ ID NO: 168 - protein, 20 aa, synthetic construct
AREPRIGYYYESSGYYSLDY

SEQ ID NO: 169 - protein, 20 aa, synthetic construct
AREPRIGYYYESSGYDSLDY

SEQ ID NO: 170 - protein, 120 aa, synthetic construct
QLQLQESGPGLVKPSETLSLTCTVSGGSISSSNYFWGWIRQSPGKGLEWIGSIDYSGYTYYNPSLKSRVTISVDTSKNQFSLKLSSVTAADTAVYNCARHKAATADFDYRGQGTLVTVSS

SEQ ID NO: 171 - protein, 127 aa, synthetic construct
QVQLVESGGGVVQPGRSLRLSCAASGFSFSRYGMHWVRQAPGKGLEGVAVIWYDGSNRYYADSVKGRFTISRDNSKNTLYLQMNSLRAEDTAVYYCAREPRIGYYYESSGYYSLDYRGQGTLVTVSS

SEQ ID NO: 172 - protein, 120 aa, synthetic construct
QLQLQESGPGLVKPSETLSLTCTVSGGSISSSSYYWGWIRQPPGKGLEWIGSIDYSGYTYYNPSLQSRVTISVDTSKNQFSLKLSSVTAADTAVYNCARHKAATADFDYRGQGTLVTVSS

SEQ ID NO: 173 - protein, 120 aa, synthetic construct
QLQLQESGPGLVKPSETLSLTCTVSGGSISSSSYYWGWIRQPPGKGLEWIGSVDYSGYTYYNPSLQSRVTISVDTSKNQFSLKLSSVTAADTAVYNCARHKAATADFDYRGQGTLVTVSS

SEQ ID NO: 174 - protein, 120 aa, synthetic construct
QLQLQESGPGLVKPSETLSLTCTVSGGSISSSSYYWGWIRQPPGKGLEWIGSIDYSGYTYYNPSLKSRVTISVDTSKNQFSLKLSSVTAADTAVYNCARHKAATADFDYRGQGTLVTVSS

SEQ ID NO: 175 - protein, 127 aa, synthetic construct
QVQLVESGGGVVQPGRSLRLSCAASGFSFRSYGMHWVRQAPGKGLEWVAVIWYDGSNKYYADSVKGRFTISRDYSKNTLYLQMNSLRAEDTAVYYCAREPRIGYYYESSGYDSLDYRGQGTLVTVSS

SEQ ID NO: 176 - protein, 127 aa, synthetic construct
QVQLVESGGGVVQPGRSLRLSCAASGFSFSSYGMHWVRQAPGKGLEWVAVIWYDGSNKYYADSVKGRFTISRDYSKNTLYLQMNSLRAEDTAVYYCAREPRIGYYYESSGYDSLDYRGQGTLVTVSS

SEQ ID NO: 177 - protein, 6 aa, synthetic construct
QSVSSN

SEQ ID NO: 178 - skipped

SEQ ID NO: 179 - protein, 9 aa, synthetic construct
QQYNNWPWT

SEQ ID NO: 180 - protein, 107 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNWPWTFGQGTKVEIK

SEQ ID NO: 181 - protein, 8 aa, synthetic construct
GFTFDDYA

SEQ ID NO: 182 - protein, 8 aa, synthetic construct
ISWNSGSI

SEQ ID NO: 183 - protein, 16 aa, synthetic construct
AKDSRGYGDYRLGGAY

SEQ ID NO: 184 - protein, 123 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDSRGYGDYRLGGAYWGQGTLVTVSS

SEQ ID NO: 185 - protein, 214 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYNNWPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 186 - protein, 450 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDSRGYGDYRLGGAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 187 - protein, 349 aa, synthetic construct
QLQLQESGPGLVKPSETLSLTCTVSGGSISSSNYFWGWIRQSPGKGLEWIGSIDYSGYTYYNPSLKSRVTISVDTSKNQFSLKLSSVTAADTAVYNCARHKAATADFDYRGQGTLVTVSSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 188 - protein, 479 aa, synthetic construct
QLQLQESGPGLVKPSETLSLTCTVSGGSISSSNYFWGWIRQSPGKGLEWIGSIDYSGYTYYNPSLKSRVTISVDTSKNQFSLKLSSVTAADTAVYNCARHKAATADFDYRGQGTLVTVSSGGGGSGGGGSQLQLQESGPGLVKPSETLSLTCTVSGGSISSSNYFWGWIRQSPGKGLEWIGSIDYSGYTYYNPSLKSRVTISVDTSKNQFSLKLSSVTAADTAVYNCARHKAATADFDYRGQGTLVTVSSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 189 - protein, 356 aa, synthetic construct
QVQLVESGGGVVQPGRSLRLSCAASGFSFSRYGMHWVRQAPGKGLEGVAVIWYDGSNRYYADSVKGRFTISRDNSKNTLYLQMNSLRAEDTAVYYCAREPRIGYYYESSGYYSLDYRGQGTLVTVSSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 190 - protein, 493 aa, synthetic construct
QVQLVESGGGVVQPGRSLRLSCAASGFSFSRYGMHWVRQAPGKGLEGVAVIWYDGSNRYYADSVKGRFTISRDNSKNTLYLQMNSLRAEDTAVYYCAREPRIGYYYESSGYYSLDYRGQGTLVTVSSGGGGSGGGGSQVQLVESGGGVVQPGRSLRLSCAASGFSFSRYGMHWVRQAPGKGLEGVAVIWYDGSNRYYADSVKGRFTISRDNSKNTLYLQMNSLRAEDTAVYYCAREPRIGYYYESSGYYSLDYRGQGTLVTVSSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 191 - protein, 486 aa, synthetic construct
QLQLQESGPGLVKPSETLSLTCTVSGGSISSSNYFWGWIRQSPGKGLEWIGSIDYSGYTYYNPSLKSRVTISVDTSKNQFSLKLSSVTAADTAVYNCARHKAATADFDYRGQGTLVTVSSGGGGSGGGGSQVQLVESGGGVVQPGRSLRLSCAASGFSFSRYGMHWVRQAPGKGLEGVAVIWYDGSNRYYADSVKGRFTISRDNSKNTLYLQMNSLRAEDTAVYYCAREPRIGYYYESSGYYSLDYRGQGTLVTVSSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 192 - protein, 486 aa, synthetic construct
QVQLVESGGGVVQPGRSLRLSCAASGFSFSRYGMHWVRQAPGKGLEGVAVIWYDGSNRYYADSVKGRFTISRDNSKNTLYLQMNSLRAEDTAVYYCAREPRIGYYYESSGYYSLDYRGQGTLVTVSSGGGGSGGGGSQLQLQESGPGLVKPSETLSLTCTVSGGSISSSNYFWGWIRQSPGKGLEWIGSIDYSGYTYYNPSLKSRVTISVDTSKNQFSLKLSSVTAADTAVYNCARHKAATADFDYRGQGTLVTVSSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK